SEQUENCE LISTING (47 sequences)

SEQ ID NO: 1 - DNA, 16078 bp, synthetic construct
  source 1..16078: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 2 - DNA, 9281 bp, synthetic construct
  source 1..9281: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgatcgatggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatgtcgacgtttaaaccatatg

SEQ ID NO: 3 - DNA, 1746 bp, synthetic construct
  source 1..1746: mol_type = other DNA
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 4 - skipped

SEQ ID NO: 5 - DNA, 738 bp, synthetic construct
  source 1..738: mol_type = other DNA
atggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgagaagtactagtggccacgtgggccgtgcaccttaa

SEQ ID NO: 6 - DNA, 490 bp, synthetic construct
  source 1..490: mol_type = other DNA
gggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatg

SEQ ID NO: 7 - DNA, 1248 bp, synthetic construct
  source 1..1248: mol_type = other DNA
atgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaa

SEQ ID NO: 8 - DNA, 253 bp, synthetic construct
  source 1..253: mol_type = other DNA
ctacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaacc

SEQ ID NO: 9 - DNA, 2429 bp, synthetic construct
  source 1..2429: mol_type = other DNA
caaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccgg

SEQ ID NO: 10 - DNA, 3006 bp, synthetic construct
  source 1..3006: mol_type = other DNA
atgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtag

SEQ ID NO: 11 - DNA, 2424 bp, synthetic construct
  source 1..2424: mol_type = other DNA
atggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtag

SEQ ID NO: 12 - skipped

SEQ ID NO: 13 - DNA, 684 bp, synthetic construct
  source 1..684: mol_type = other DNA
atgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactga

SEQ ID NO: 14 - DNA, 353 bp, synthetic construct
  source 1..353: mol_type = other DNA
atgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtc

SEQ ID NO: 15 - DNA, 387 bp, synthetic construct
  source 1..387: mol_type = other DNA
atggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 16 - DNA, 393 bp, synthetic construct
  source 1..393: mol_type = other DNA
atgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatga

SEQ ID NO: 17 - DNA, 351 bp, synthetic construct
  source 1..351: mol_type = other DNA
atgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataa

SEQ ID NO: 18 - DNA, 345 bp, synthetic construct
  source 1..345: mol_type = other DNA
atggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtag

SEQ ID NO: 19 - DNA, 103 bp, synthetic construct
  source 1..103: mol_type = other DNA
aatcatcaataatataccttattttggattgaagccaatatgataatgagggggtggagtttgtgacgtggcgcggggcgtgggaacggggcgggtgacgtag

SEQ ID NO: 20 - DNA, 229 bp, synthetic construct
  source 1..229: mol_type = other DNA
cacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagc

SEQ ID NO: 21 - DNA, 626 bp, synthetic construct
  source 1..626: mol_type = other DNA
cacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaatt

SEQ ID NO: 22 - DNA, 2600 bp, synthetic construct
  source 1..2600: mol_type = other DNA
ggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaag

SEQ ID NO: 23 - DNA, 2637 bp, synthetic construct
  source 1..2637: mol_type = other DNA
agggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttattttt

SEQ ID NO: 24 - DNA, 1064 bp, synthetic construct
  source 1..1064: mol_type = other DNA
aatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagc

SEQ ID NO: 25 - DNA, 84 bp, synthetic construct
  source 1..84: mol_type = other DNA
ttaatgtagttgtggccagaccagtcccatgaaaatgacatagagtatgcacttggagttgtgtctcctgtttcctgtgtaccg

SEQ ID NO: 26 - DNA, 16 bp, synthetic construct
  source 1..16: mol_type = other DNA
gaagcgcgcaagaccg

SEQ ID NO: 27 - DNA, 19 bp, synthetic construct
  source 1..19: mol_type = other DNA
cggataggcgcaaagagag

SEQ ID NO: 28 - DNA, 26 bp, synthetic construct
  source 1..26: mol_type = other DNA
acggaaaccggtcctccaactgtgcc

SEQ ID NO: 29 - DNA, 26 bp, synthetic construct
  source 1..26: mol_type = other DNA
cggagacaaaactaaacctgtaacac

SEQ ID NO: 30 - DNA, 18 bp, synthetic construct
  source 1..18: mol_type = other DNA
ttgtggccagaccagtcc

SEQ ID NO: 31 - DNA, 31 bp, synthetic construct
  source 1..31: mol_type = other DNA
acggtacacaggaaacaggagacacaactcc

SEQ ID NO: 32 - DNA, 18 bp, synthetic construct
  source 1..18: mol_type = other DNA
agcgcgcgaataaactgc

SEQ ID NO: 33 - DNA, 20 bp, synthetic construct
  source 1..20: mol_type = other DNA
taagtgagatcagggtgcgc

SEQ ID NO: 34 - DNA, 26 bp, synthetic construct
  source 1..26: mol_type = other DNA
cgctccgtcctgcaggaatacaacat

SEQ ID NO: 35 - DNA, 615 bp, synthetic construct
  source 1..615: mol_type = other DNA
atgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaa

SEQ ID NO: 36 - DNA, 972 bp, synthetic construct
  source 1..972: mol_type = other DNA
atgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaa

SEQ ID NO: 37 - DNA, 12852 bp, synthetic construct
  source 1..12852: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 38 - DNA, 13441 bp, synthetic construct
  source 1..13441: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggccaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 39 - DNA, 10423 bp, synthetic construct
  source 1..10423: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaaggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 40 - DNA, 15847 bp, synthetic construct
  source 1..15847: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagtagaagcgcgcaagaccgtcttaagataccttcaaccccgtgtaaccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaataggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctataggtcgcgggcaacacactcacctagcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaattagcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctaggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactcttagtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaacctagtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatctagattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacaagtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagcctaaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 41 - DNA, 16076 bp, synthetic construct
  source 1..16076: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 42 - DNA, 12675 bp, synthetic construct
  source 1..12675: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcacaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccaaggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatacatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcagggattagtcccgcgcgcgcacacgtggcggccgccgacctggtaaccgcatacgagcagacggtgaaccaggagattaactttcaaaaaagctttaaggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttactaatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgctttaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgactaaatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctatagcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctaacagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagccaatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggacatgtttcgcgccctttctcaaatttaagcgcgaaaactacgtcatctccagcggccacacccggcgccagcacctgttgtcagcgccattttgagcaaggaaattcccacgcccatctagtggagttaccagccacaatagggacttgcggctggagctgcccaagactactcaacccgaataaactactagagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagtagaagcgcgcaagaccgtcttaagataccttcaaccccgtgtaaccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaataggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctataggtcgcgggcaacacactcacctagcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaattagcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctaggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactcttagtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaacctagtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatctagattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacaagtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagcctaaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 43 - DNA, 15104 bp, synthetic construct
  source 1..15104: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcacaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccaaggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatacatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcagggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttactaatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgctttaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgactaaatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctatagcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctaacagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagccaatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggacatgtttcgcgccctttctcaaatttaagcgcgaaaactacgtcatctccagcggccacacccggcgccagcacctgttgtcagcgccattttgagcaaggaaattcccacgcccatctagtggagttaccagccacaatagggacttgcggctggagctgcccaagactactcaacccgaataaactactagagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagtagaagcgcgcaagaccgtcttaagataccttcaaccccgtgtaaccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaataggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctataggtcgcgggcaacacactcacctagcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaattagcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctaggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactcttagtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaacctagtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatctagattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacaagtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagcctaaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 44 - DNA, 12906 bp, synthetic construct
  source 1..12906: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcacaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccaaggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatacatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcagggattagtcccgcgcgcgcacacgtggcggccgccgacctggtaaccgcatacgagcagacggtgaaccaggagattaactttcaaaaaagctttaaggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttactaatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgctttaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgactaaatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctatagcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctaacagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagccaatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggacatgtttcgcgccctttctcaaatttaagcgcgaaaactacgtcatctccagcggccacacccggcgccagcacctgttgtcagcgccattttgagcaaggaaattcccacgcccatctagtggagttaccagccacaatagggacttgcggctggagctgcccaagactactcaacccgaataaactactagagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagtagaagcgcgcaagaccgtcttaagataccttcaaccccgtgtaaccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 45 - DNA, 12675 bp, synthetic construct
  source 1..12675: mol_type = other DNA
gcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcacaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccaaggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatacatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcagggattagtcccgcgcgcgcacacgtggcggccgccgacctggtaaccgcatacgagcagacggtgaaccaggagattaactttcaaaaaagctttaaggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagtagaagcgcgcaagaccgtcttaagataccttcaaccccgtgtaaccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaataggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctataggtcgcgggcaacacactcacctagcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaattagcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctaggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactcttagtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaacctagtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatctagattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacaagtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagcctaaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgc

SEQ ID NO: 46 - DNA, 8908 bp, synthetic construct
  source 1..8908: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccattcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatcgccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccattgttgggggacgtcgcgccgcaccgcgtccgcgctcgggggtggtttcgcgctgctcctcttcccgactggccatttccttctcctataggcagaaaaagatcgagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgactaataagagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctagcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattgcccgggcgttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttgctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttcagaaacccgcagactttgagagaaaagtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcattcgctgcttgaggctgaaggtggagggcgctctggagcagatttttacagccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacgttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgatcgatggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatgtcgacgtttaaaccatatg

SEQ ID NO: 47 - DNA, 7781 bp, synthetic construct
  source 1..7781: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccattcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatcgccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccattgttgggggacgtcgcgccgcaccgcgtccgcgctcgggggtggtttcgcgctgctcctcttcccgactggccatttccttctcctataggcagaaaaagatcgagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgactaataagagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctagcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattgcccgggcgttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgttcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcaatcgatggatgtcgcccctcctgacgcggtaggaggaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatgtcgacgtttaaaccatatg

(19) United States
(12) Patent Application Publication
(10) Pub. No.: US 2026/0117201 A1
(43) Pub. Date: Apr. 30, 2026

(54) Polynucleotides for use in AAV production

(71) Applicant: ENCODED THERAPEUTICS, INC., South San Francisco, CA (US)

(72) Inventors: Anne Tanenhaus, South San Francisco, CA (US); Tulasi Solanki, South San Francisco, CA (US); Adam Miller, San Carlos, CA (US); Ulrike Jung, Ulm (DE)

(21) Appl. No.: 19/375,179

(22) Filed: Oct. 30, 2025

Related U.S. Application Data
(60) Provisional application No. 63/714,463, filed on Oct. 31, 2024.

(51) Int. Cl. C12N 7/00; C07K 14/005

(52) U.S. Cl. CPC C12N 7/00; C07K 14/005; C12N 2710/10322; C12N 2750/14122; C12N 2750/14143; C12N 2750/14152; C12N 2750/14162; C12N 2800/40

(57) ABSTRACT

The disclosure provides adeno-associated virus (AAV) helper plasmid variants with improved safety profiles. In certain aspects, an AAV helper plasmid variant of the disclosure comprises one or more inactivating mutations in at least one of the following adenovirus genes or genome sequences: (a) adenoviral fiber gene; (b) adenoviral precursor terminal protein gene; (c) adenoviral L1-52K gene; (d) adenoviral 100K gene; (e) adenoviral PVIII gene; (f) adenoviral E4 region open reding frame; (g) adenoviral inverted terminal repeat (ITR) sequence; (h) L3-23K region gene; (i) hexon-assembly gene; or (j) a combination of any of (a)-(i).

### CROSS-REFERENCE TO RELATED APPLICATIONS

This application claims priority to U.S. Provisional Patent Application No. 63/714,463, filed Oct. 31, 2024, which is hereby incorporated by reference in its entirety.

### TECHNICAL FIELD

The disclosure relates to adeno-associated virus helper plasmids and methods of use.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 227,831 byte .XML file named “57094P_Seqlisting.xml”; created on Oct. 28, 2024.

### BACKGROUND

Adeno-associated virus (AAV) is a small, replication-defective, non-enveloped animal virus belonging to the family Parvoviridae. The AAV genome consists of a linear single stranded DNA which is ˜4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion.

Due to the specificity, efficiency, and safety associated with AAV, AAV vectors have emerged as the expression vector of choice for gene therapy applications. Manufacturing of AAV vectors for administration to patients presents multiple challenges, however. In nature, AAV is typically accompanied by adenovirus in productive infection, requiring the function of various adenovirus genes to replicate and package. AAV vector manufacturing techniques leverage the functions of adenovirus genes during vector production by introducing adenoviral genes alongside the AAV vector backbones in cell culture, e.g., as a helper virus, in one or more helper plasmid vector, and/or integrated into the host cell genome. While providing adenovirus genes allows efficient production of packaged AAV virions, it presents a safety issue with respect to the presence of adenovirus-derived genes/open reading frames that are packaged into viral particles and/or contaminating adenoviral proteins in the resulting pharmaceutical composition. There is thus a need in the art for materials and methods for efficient production of AAV vectors which minimizes safety concerns relating to helper virus-originated contamination.

### SUMMARY

In certain aspects, the disclosure provides polynucleotides comprising one or more inactivated adenoviral genes. In certain embodiments, the polynucleotide is in the form of a plasmid, where in some cases the plasmid is an adeno-associated virus (AAV) helper plasmid. In certain embodiments, the AAV helper plasmid, also referred to as an AAV helper plasmid variant, has an improved safety profile as compared to a parent AAV helper plasmid that does not include the inactivated adenoviral gene. In certain aspects, an AAV helper plasmid variant of the disclosure comprises one or more inactivating mutations in at least one of the following adenovirus genes or genome sequences: (a) adenoviral fiber gene; (b) adenoviral precursor terminal protein (pTP) gene; (c) adenoviral L1-52K region gene; (d) adenoviral 100K gene; (e) adenoviral PVIII gene; (f) one or more adenoviral E4 region open reading frames (E4orf1 to E4orf4); (g) adenoviral inverted terminal repeat (ITR) sequence; (h) L3-23K region gene; (i) hexon-assembly gene; or (j) a combination of any of (a)-(i). In certain aspects, the one or more inactivating mutations are selected from Table 2. In some embodiments, the AAV helper plasmid variant includes the combination of inactivating mutations listed for any one of the A1 to A9, B1 or B1 variants shown in Table 3, where in some embodiments such an AAV helper plasmid variant has at least 80% sequence identity to any one of SEQ ID NOs: 37 to 47.

The disclosure further provides a plasmid system comprising an AAV helper plasmid variant as described herein and (i) a plasmid comprising a nucleic acid encoding an AAV Rep protein and a nucleic acid encoding an AAV Cap protein or (ii) a plasmid comprising a nucleic acid encoding an AAV Rep protein and a plasmid comprising a nucleic acid encoding an AAV Cap protein. Optionally, the system further comprises a plasmid comprising at least one heterologous nucleic acid, wherein the heterologous nucleic acid(s) is flanked by a 5′ and 3′ AAV inverted terminal repeat (ITR). Also provided is a host cell comprising an AAV helper plasmid variant described herein or the plasmid system described herein. Further provided is a method of producing recombinant AAV, the method comprising culturing the host cell and isolating recombinant AAV.

Preferred embodiments of this disclosure are described herein, including the best mode known to the inventors for carrying out the disclosure. Variations of those preferred embodiments may become apparent to those of ordinary skill in the art upon reading the foregoing description. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Indeed, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document.

### BRIEF DESCRIPTION OF THE DRAWINGS

FIG. 1 is a schematic alignment of AAV helper plasmid Variants A1 to A9 described herein (SEQ ID NOs: 37 to 45, respectively) with Parent A (SEQ ID NO:1). The plasmid backbone elements are not included in FIG. 1 or the SEQ ID NOs (e.g., the origin of replication, f1, antibiotic resistance gene, etc.). Relevant features present in Parent A and their location are indicated below the alignment, with arrows indicating directionality of the feature (e.g., direction of transcription). The helper plasmid designations for each plasmid variant are provided at the left (variant A1, variant A2, and so on). Hashes in the aligned regions represent point mutations or small insertions/deletions, whereas open regions (i.e., white blocks) represent larger deletions. This figure aligns the helper plasmids in a linear format to aid in showing their structural features and location of inactivation mutations even though they are circular plasmids when used for AAV production experiments in the Examples.

FIG. 2 is a schematic alignment of AAV helper plasmid Variants B1 and B2 described herein (SEQ ID NOs: 46 and 47, respectively) with Parent B (SEQ ID NO:2). The plasmid backbone elements are not included in FIG. 2 or the SEQ ID NOs (e.g., the origin of replication, f1, antibiotic resistance gene, etc.). Relevant features present in Parent B and their location are indicated below the alignment, with arrows indicating directionality of the feature (e.g., direction of transcription). The helper plasmid designations for each variant plasmid are provided at the left (variant B1 and variant B2). Hashes in the aligned regions represent point mutations or small insertions/deletions, whereas open regions (i.e., white blocks) represent larger deletions. This figure aligns the helper plasmids in a linear format to aid in showing their structural features and location of inactivation mutations even though they are circular plasmids when used for AAV production experiments in the Examples.

FIGS. 3A and 3B are bar graphs illustrating AAV production and packaging efficiency of variant AAV helper plasmids with fiber gene deletions (variants A1, A2, and A3) as compared to their parent helper plasmid (Parent A) at shake flask scale (30 mL). The helper plasmid is identified on the x-axis. The % viral genome per mL (Vg/mL) relative to a Parent Ais represented on the y-axis in FIG. 3A. The ratio of capsid protein to viral genome (Cp:Vg) is represented on the y-axis in FIG. 3B. The helper constructs are described in Table 2, which notes the Ad gene inactivating mutations for each variant.

FIGS. 4A and 4B are bar graphs illustrating AAV production and packaging efficiency of variant AAV helper plasmids described herein (variants A4 to A9) as compared to their parent helper plasmid (Parent A) at shake flask scale (30 mL). The helper plasmid is identified on the x-axis. The % viral genome per mL (Vg/mL) relative to a Parent A is represented on the y-axis in FIG. 4A. The ratio of capsid protein to viral genome (Cp:Vg) is represented on the y-axis in FIG. 4B. The helper constructs are described in Table 2, which notes the Ad gene inactivating mutations for each variant.

FIGS. 5A and 5B are bar graphs illustrating AAV production and packaging efficiency of variant AAV helper plasmids described herein (variants B1 and B2) as compared to their parent helper plasmid (Parent B) at shake flask scale (30 mL). The helper plasmid is identified on the x-axis. The % viral genome per mL (Vg/mL) relative to a Parent B is represented on the y-axis in FIG. 5A. The ratio of capsid protein to viral genome (Cp:Vg) is represented on the y-axis in FIG. 5B. The helper constructs are described in Table 2, which notes the Ad gene inactivating mutations for each variant. Notably, Variant B2 provides an adenoviral titer substantially equivalent to the parent plasmid (Parent B) and similar packaging efficiency (i.e., similar Cp:Vg ratio) while lacking multiple adenoviral open reading frames (ORFs) that are present in Parent B.

FIGS. 6A and 6B are bar graphs illustrating AAV production and packaging efficiency of variant AAV helper plasmids described herein (variants A4, A5, and A9) as compared to their parent helper plasmid (Parent A) at bioreactor scale (2.0 L). The helper plasmid is identified on the x-axis. The % viral genome per mL (Vg/mL) relative to Parent B is represented on the y-axis in FIG. 6A. The ratio of capsid protein to viral genome (Cp:Vg) is represented on the y-axis in FIG. 6B. The helper constructs are described in Table 2, which notes the Ad gene inactivating mutations for each variant.

FIGS. 7A and 7B are bar graphs illustrating AAV production and packaging efficiency of variant B2 as compared to Parent B at bioreactor scale (2.0 L). The helper plasmid is identified on the x-axis. The % viral genome per mL (Vg/mL) relative to a Parent B is represented on the y-axis in FIG. 7A. The ratio of capsid protein to viral genome (Cp:Vg) is represented on the y-axis in FIG. 7B. The helper constructs are described in Table 2, which notes the Ad gene inactivating mutations for variant B2.

FIG. 8 is a graph illustrating relative fiber mRNA expression from variant AAV helper plasmids described herein using an RT-PCR assay. Two separate primer sets were used: one targeting a 5′ region of the fiber mRNA transcript and one targeting a 3′ region of the fiber transcript. The region targeted is indicated at the top of the graph. The helper plasmids are noted on the x-axis. The relative fiber mRNA expression is noted on the y-axis and is calculated relative to the level of fiber mRNA expression from Parent A, which has no fiber gene inactivating mutations (i.e., Parent A is a positive control). Variant B2, which lacks most of the fiber gene (see FIG. 2), is provided as a negative control. Variants A4, A6, and A9 comprise fiber gene inactivating mutations that result in significantly reduced fiber mRNA transcript expression.

### DETAILED DESCRIPTION

As summarized above, the disclosure provides polynucleotides comprising one or more inactivated adenoviral genes and methods of using the same. In some embodiments, the polynucleotides are employed for the production of recombinant AAV stocks for use in gene therapy with improved safety profiles, e.g., in the form of an AAV helper plasmid variant. In particular, the materials and methods described herein allow for an AAV vector drug substance production process that reduces contaminants derived from industry-standard AAV helper plasmids without significantly reducing AAV vector yield. In various aspects, the disclosure provides an adeno-associated virus (AAV) helper plasmid comprising one or more inactivating mutations in at least one of the following adenovirus genes or genome sequences: (a) adenoviral fiber gene; (b) adenoviral precursor terminal protein (pTP) gene; (c) adenoviral L1-52K region gene; (d) adenoviral 100K gene; (e) adenoviral PVIII gene; (f) one or more adenoviral E4 open reading frames (E4orf1 to E4orf4); (g) adenoviral inverted terminal repeat (ITR) sequence; (h) L3-23K region gene, and/or (i) hexon-assembly gene, as well as (j) combinations of any of (a)-(i).

An AAV helper plasmid comprises adenoviral nucleic acids which provide the adenovirus-related functions required for propagation of AAV virus in a host cell. In the context of the disclosure, the AAV helper plasmid, at a minimum, comprises Ad E4ORF6, as well as nucleic acids encoding VA1, VA2, and Ad DNA binding protein (DBP). In various aspects of the disclosure, the AAV helper plasmid further comprises one or more adenoviral genes (or coding sequences that encode the corresponding adenoviral protein) or functional nucleic acid sequences, such as the Ad fiber gene, the Ad precursor terminal protein gene, the Ad L1-52K gene, the Ad 100K protein gene, the Ad PVIII protein gene, Ad E4 regions (e.g., Ad E4ORF1, Ad E4ORF2, Ad E4ORF3, and/or Ad E4ORF4), then hexon-assembly gene, the Ad L3-23K region protein gene, and/or Ad terminal repeat (ITR) sequence(s). In various aspects of the disclosure, the AAV helper plasmid further comprises Ad E1a and/or Ad E1b coding sequences.

“Gene” refers to a nucleic acid region that is capable of expressing an RNA molecule (sometimes referred to as a transcript) under certain conditions. The RNA molecule can be, for example, an mRNA that encodes a protein, a functional RNA (e.g., an antisense RNA, ribozyme, a microRNA, etc.), or a combination of thereof. In some aspects, a gene comprises one or more regulatory elements operably linked to the RNA-expressing region that play a role in controlling its expression. The regulatory elements can be oriented with respect to the RNA-expressing region in any manner that allows them to exert their regulatory function, and thus can precede, follow, and/or be interspersed within the coding region. “Operably-linked” refers to the physical association of two or more nucleic acid sequence elements such that the function of one of the sequences is affected by another. Regulatory elements include, but are not limited to, promoters, enhancers, polyadenylation sequences,‘5’-untranslated regions,‘3’-untranslated regions, introns, and the like.

“Inactivation mutations” of the present disclosure include any mutation to a gene that decreases, reduces, or inhibits RNA expression from the gene and/or the production of the full-length or functional form of a protein encoded by the gene. In various aspects, the type of inactivation mutation(s) for a gene can include a frame-shift mutation, a start codon disruption, an internal start codon disruption, a stop codon insertion, a deletion, an insertion, an inversion, or any combination thereof. As used herein, “one or more inactivating mutations in at least one of the following adenovirus genes or genome sequences” refers to inactivating mutations within an expressed RNA region itself, e.g., a coding region, or within associated gene regulatory elements such that expression of the RNA is reduced. In some aspects, an inactivating mutation may be within a non-gene region, such as an adenoviral ITR sequence. A single inactivation mutation may be categorized in multiple ways. For example, a frame-shift mutation at a first location in a coding region can result in a new stop codon appearing in the coding region downstream of the frame-shift mutation, functionally resembling a stop codon insertion at that downstream site. Such categorizations of inactivation mutations are well understood in the art and underscore that the specific inactivation mutations disclosed herein are meant to be examples and thus should not be considered as limiting the scope of this disclosure. Examples of inactivating mutations are described below in Table 2, and the disclosure contemplates AAV helper plasmids comprising any one or more of the mutations described in Table 2. Various mutations are described herein with respect to locations within a reference nucleic acid sequence. For example, inactivating mutations with respect to the Ad fiber gene are described herein with reference to SEQ ID NO: 3, which corresponds to the wild type human adenovirus serotype 5 (HAdV-5, or Ad5) fiber gene. One of ordinary skill will appreciate that the disclosure is not limited to a particular adenoviral serotype (unless the context of a particular description dictates otherwise). The reference sequence is provided merely to provide context for the nucleotide position of the mutations. The sequences of adenoviral genomes of different serotypes (e.g., HAdV serotype 2, or Ad2) are known in the art, and one of ordinary skill need only to compare alignments of a gene sequence of particular serotype with the reference sequence provided herein to determine corresponding positions for mutations in other (i.e., non-Ad5) serotype genes. In various aspects, one or more of the adenovirus genes are human adenovirus serotype 5 (Ad5) genes. Optionally, one or more of the adenovirus genes are human adenovirus serotype 2 (Ad2) genes.

In various aspects, a polynucleotide of the present disclosure, e.g., an AAV helper plasmid variant, comprises an adenoviral fiber gene comprising one or more inactivating mutations, sometimes referred to herein as an “inactivated adenoviral fiber gene” or an “inactivated fiber gene”. For example, an inactivated adenoviral fiber gene includes at least one inactivating mutation that prevents expression of the protein encoded by the fiber gene, e.g., a frame-shift mutation, mutation of the start codon, a deletion, etc., as described herein. In certain embodiments, the adenoviral fiber gene comprises a sequence having at least 95% sequence identity to SEQ ID NO: 3. In some embodiments, the inactivated fiber gene comprises one or more of the following mutations: (i) deletion of A at nucleotide position 49 and deletion of T at nucleotide position 50 (c.49delAT); (ii) conversion of AT at nucleotide positions 1 and 2 to TA, converting the ATG start codon to TAG stop codon (c.1_2AT>TA); (iii) a G to T mutation at nucleotide position 22 to generate a premature stop codon (c.22G>T); (iv) a T to A mutation at nucleotide position 45 (c.45T>A); (v) deletion of A at nucleotide position 196 and a point mutation at position 198 to convert G to A, which generates a frameshift and premature stop codon (c.196_199ATGG>TAG); (vi) conversion of AT at nucleotide positions 361 and 362 to destroy an internal Met codon (c.361-362AT>TA); (vii) conversion of AT at nucleotide positions 385 and 386 to destroy an internal Met codon (c.385-386AT>TA); (viii) a conversion of AT at nucleotide positions 751 and 752 to destroy an internal Met codon (c.751-752AT>TA); (ix) a deletion of T at nucleotide position 1118 to produce frameshift mutation (c.1118delT); (x) conversion of AT at nucleotide positions 1654 and 1655 to destroy an internal Met codon (c.1654-1655AT>TA); and/or (xi) deletion of SEQ ID NO: 4, which removes residual non-coding fiber sequence from parent B (FiberDeletion3). The nucleotide positions of the fiber gene inactivating mutations are in reference to SEQ ID NO: 3.

In various aspects, a polynucleotide of the present disclosure, e.g., an AAV helper plasmid variant, comprises an adenoviral precursor terminal protein (pTP) gene comprising one or more inactivation mutations, sometimes referred to herein as an “inactivated adenoviral pTP gene” or an “inactivated pTP gene.” For example, an inactivated pTP gene includes at least one inactivating mutation that prevents expression of the protein encoded by the pTP gene, e.g., a frame-shift mutation, mutation of the start codon, a deletion, etc., as described herein. In certain embodiments, the adenoviral pTP gene comprises a nucleic acid sequence at least 95% identical to SEQ ID NO: 5. In some embodiments, the inactivated adenoviral pTP gene comprises a mutation at position 1 which converts T to A, thereby destroying the pTP start codon (c.1T>A mutation), and/or deletion of SEQ ID NO: 6 to remove pTP ORF sequence (pTPDeletion1). The nucleotide positions of the adenoviral pTP gene inactivating mutations are in reference to SEQ ID NO: 5, which is the wild type Ad5 adenoviral pTP gene sequence.

In various aspects, a polynucleotide of the present disclosure, e.g., an AAV helper plasmid variant, comprises an adenoviral L1-52K gene comprising one or more inactivation mutations, sometimes referred to herein as an “inactivated adenoviral L1-52K gene” or an “inactivated L1-52K gene”. For example, an inactivated L1-52K gene includes at least one inactivating mutation that prevents expression of the protein encoded by the L1-53K gene, e.g., a frame-shift mutation, mutation of the start codon, a deletion, etc., as described herein. In certain embodiments, the adenoviral L1-52K gene comprises a nucleic acid sequence at least 95% identical to SEQ ID NO: 7. In some embodiments, the inactivated adenoviral L1-52K gene comprises a point mutation at nucleotide position 3 to convert G to A, thereby destroying the L1-52K start codon (c.3G>A); and/or deletion of SEQ ID NO: 8 to remove the L1-52K ORF sequence (L1-52K Deletion1). In some embodiments, the L1-52K gene and additional downstream open reading frames (orfs) are inactivated by deletion of SEQ ID NO: 9 (removes significant portion of the L1-52K gene (SEQ ID NO: 7), all of the pllla gene (SEQ ID NO: 36), and a significant portion of the L3-23K endoprotease gene (SEQ ID NO: 35) (L1-52K Deletion2)). The nucleotide positions of the adenoviral L1-52K gene inactivating mutations are in reference to SEQ ID NO: 7, which is the wild type Ad5 adenoviral L1-52K gene.

In various aspects, a polynucleotide of the present disclosure, e.g., an AAV helper plasmid variant, comprises an adenoviral 100K gene comprising one or more inactivation mutations, sometimes referred to herein as an “inactivated adenoviral 100K gene” or an “inactivated 100K gene”. For example, an inactivated 100K gene includes at least one inactivating mutation that prevents expression of the protein encoded by the 100K gene, e.g., a frame-shift mutation, mutation of the start codon, a deletion, etc., as described herein. In certain embodiments, the adenoviral 100K gene comprises a nucleic acid sequence at least 95% identical to SEQ ID NO: 10. In some embodiments, the inactivated adenoviral 100K gene comprises one or more of the following mutations: (i) a point mutation converting T to A at nucleotide position, creating a nonsense mutation to generate premature stop codon (c.1847T>A); (ii) a point mutation converting T at nucleotide position 2026 to A, creating a nonsense mutation to generate premature stop codon (c.2026A>T); (iii) a point mutation converting A to T at nucleotide position 2239 and a point mutation converting T to A at position 2240, creating a nonsense mutation to generate premature stop codon (c.2239_2240AT>TA); (iv) a point mutation converting A to T at nucleotide position 2519 and a point mutation converting T to A at position 2520, creating a nonsense mutation to generate premature stop codon (c.2519_2520AT>TA); (v) a point mutation converting T to A at nucleotide position 2685, creating a nonsense mutation to generate premature stop codon (c.2685T>A); (vi) a point mutation converting G to T at nucleotide position 3, which destroys a start codon (c.3G>T); (vii) a point mutation converting G to C at nucleotide position 312, which destroys downstream 100K MET codon (c.312G>C); and/or (viii) a point mutation converting G to T at nucleotide position 480, which destroys downstream 100K MET codon (c.480G>T). The nucleotide positions of the adenoviral 100K gene inactivating mutations are in reference to SEQ ID NO: 10, which is the wild type Ad5 100K gene.

In various aspects, a polynucleotide of the present disclosure, e.g., an AAV helper plasmid variant, comprises an adenoviral hexon-assembly gene comprising one or more inactivation mutations, sometimes referred to herein as an “inactivated adenoviral hexon-assembly gene” or an “inactivated hexon-assembly gene”. For example, an inactivated hexon-assembly gene includes at least one inactivating mutation that prevents expression of the protein encoded by the hexon-assembly gene, e.g., a frame-shift mutation, mutation of the start codon, a deletion, etc., as described herein. In certain embodiments, the adenoviral hexon-assembly gene comprises a nucleic acid sequence at least 95% identical to SEQ ID NO: 11. The inactivated adenoviral hexon-assembly gene comprises one or more of the following mutations: (i) deletion of ATG at nucleotide positions 1-3, removing a start codon to destroy hexon assembly start codon (c.1_3delATG); (ii) addition of TAATAA (SEQ ID NO: 12) at nucleotide position 993, inserting a tandem stop codon to prevent hexon assembly expression (c.933insTAATAA); and/or (iii) deletion of AT at nucleotide positions 1937-1938, destroying an L4/22K potential ORF start codon (c.1937-1938delAT). The nucleotide positions of the adenoviral hexon assembly gene inactivating mutations are in reference to SEQ ID NO: 11, which is the wild type Ad5 hexon assembly gene.

In various aspects, a polynucleotide of the present disclosure, e.g., an AAV helper plasmid variant, comprises an adenoviral PVIII gene comprising one or more inactivation mutations, sometimes referred to herein as an “inactivated adenoviral PVIII gene” or an “inactivated PVIII gene”. For example, an inactivated PVIII gene includes at least one inactivating mutation that prevents expression of the protein encoded by the PVIII gene, e.g., a frame-shift mutation, mutation of the start codon, a deletion, etc., as described herein. In certain embodiments, the adenoviral PVIII gene comprises a nucleic acid sequence at least 95% identical to SEQ ID NO: 13. The inactivated adenoviral PVIII gene comprises one or more of the following mutations: (i) a point mutation converting T to A at nucleotide position 1, creating a nonsense mutation to destroy start codon (c.1T>A); (ii) a mutation converting the sequence TACAT at nucleotide positions 25-29 to ATCTA, converting an internal Met codon to a stop codon (c.25_29TACAT>ATCTA); (iii) a point mutation converting T to A at nucleotide position 49 and a point mutation converting A to T at position 50, converting an internal Met codon to a stop codon (c.49_50TA>AT); (iv) a point mutation converting A to T at nucleotide position 100 and a point mutation converting T to A at position 101, converting an internal Met codon to a stop codon (c.100_101AT>TA); and/or (v) deletion of SEQ ID NO: 14, removing Hexon-associated precursor/pVIII sequence (pVIIIDeletion1). The nucleotide positions of the adenoviral PVIII gene inactivating mutations are in reference to SEQ ID NO: 13, which is the wild type Ad5 PVIII gene.

In various aspects, a polynucleotide of the present disclosure, e.g., an AAV helper plasmid variant, comprises one or more inactivated adenoviral E4 region open reading frames (E4orf1, E4orf2, E4orf3, and/or E4orf4) comprising one or more inactivation mutations, sometimes referred to herein as an “inactivated adenoviral E4 region orf”, “inactivated E4 region orf”, an “inactivated adenoviral E4orfx” or “inactivated E4orfx”, where x is 1 to 4. For example, an inactivated E4 region includes at least one inactivating mutation that prevents expression of the protein encoded by any one or more of the E4 orfs, e.g., a frame-shift mutation, mutation of the start codon, a deletion, etc., as described herein. In certain embodiments, the adenoviral E4orf1, E4orf2, E4orf3, and E4orf4 genes comprise nucleic acid sequences at least 95% identical to SEQ ID NOs: 15, 16, 17, and 18, respectively. In some embodiments, the inactivated adenoviral E4 region protein gene comprises one or more of the following mutations: (i) mutation converting T to A at nucleotide position 1 and mutation converting A to T at nucleotide position 2 to destroy the E4orf1 start codon (E4orf1 c.1_2TA>AT); (ii) mutation converting T to A at nucleotide position 1 to destroy the E4orf2 start codon (E4orf2 c.1T>A); (iii) mutation converting T to A at nucleotide position 1 and mutation converting A to T at nucleotide position 2 to destroy E4orf3 start codon (E4orf3 c.1_2TA>AT); (iv) mutation converting T to A at nucleotide position and mutation converting A to T at nucleotide position 2 to destroy E4orf4 start codon (E4orf4 c.1_2TA>AT); (iv) deletion of ATG at nucleotide positions 1-3, removing start codon of E4orf1 (modification is in frame to avoid E4 transcript degradation) (E4orf1 c.1_3delATG); (v) deletion of SEQ ID NO:24, removing E4orf6 intron sequence containing E4orf1, E4orf2, E4orf3, E4orf4 start codons (E4Deletion1); (vi) deletion of ATG at nucleotide positions 18-16 of E4orf2, removing upstream Met (modification is in frame to avoid E4 transcript degradation) (E4orf2 c.-18_-16delATG (upstream); (vii) deletion of ATG at nucleotide positions 1-3 of E4orf2, removing E4orf2 start codon (modification is in frame to avoid E4 transcript degradation) (E4orf2 c.1_3delATG); (viii) deletion of ATG at nucleotide positions 16-18 of E4orf2, removing downstream Met (modification is in frame to avoid E4 transcript degradation) (E4orf2 c.16_18delATG); (ix) deletion of ATG at nucleotide positions 1-3 of E4orf3, removing E4orf3 start codon (modification is in frame to avoid E4 transcript degradation) (E4orf3 c.1_3delATG); (x) deletion of ATG at nucleotide positions 55-57 of E4orf3, removing E4orf3 downstream Met (modification is in frame to avoid E4 transcript degradation) (E4orf3 c.55_57delATG); and/or (xi) deletion of ATG at nucleotide positions 1-3 of E4orf4, removing E4orf4 start codon (modification is in frame to avoid E4 transcript degradation) (E4orf4 c.1_3delATG). The nucleotide positions relating to E4orf1 are in reference to SEQ ID NO: 15, which is the wild type Ad5 E4orf1 sequence. The nucleotide positions relating to E4orf2 are in reference to SEQ ID NO: 16, which is the wild type Ad5 E4orf2 sequence. The nucleotide positions relating to E4orf3 are in reference to SEQ ID NO: 17, which is the wild type Ad5 E4orf3 sequence. The nucleotide positions relating to E4orf4 are in reference to SEQ ID NO: 18, which is the wild type Ad5 E4orf4 sequence.

In various aspects, a polynucleotide of the present disclosure, e.g., an AAV helper plasmid variant, lacks all or part of an adenoviral inverted terminal repeat (ITR). For example, an AAV helper plasmid variant of the present disclosure lacks SEQ ID NO: 19 (Ad5 ITR), SEQ ID NO: 20 (ITRDeletion1) or SEQ ID NO: 21 (ITRDeletion2).

The present disclosure contemplates the use of any one or any combination of the inactivating mutations detailed above in constructing an AAV helper plasmid and/or engineering a host cell (e.g., by stable integration of one or more of the inactivated adenoviral genes/regions disclosed herein) that improves the safety profile of an AAV vector drug substance for use as a gene therapy vector in a mammalian subject, e.g., a human subject. Moreover, the present application contemplates the use of inactivating mutations in the genes/regions that are not explicitly disclosed but that result in the inactivation of the genes/regions above in a similar manner, e.g., by introducing mutations resulting in one or more of frame shifts, stop codons, start codon removal, deletions, etc., that inactivate the gene(s)/region(s) of interest.

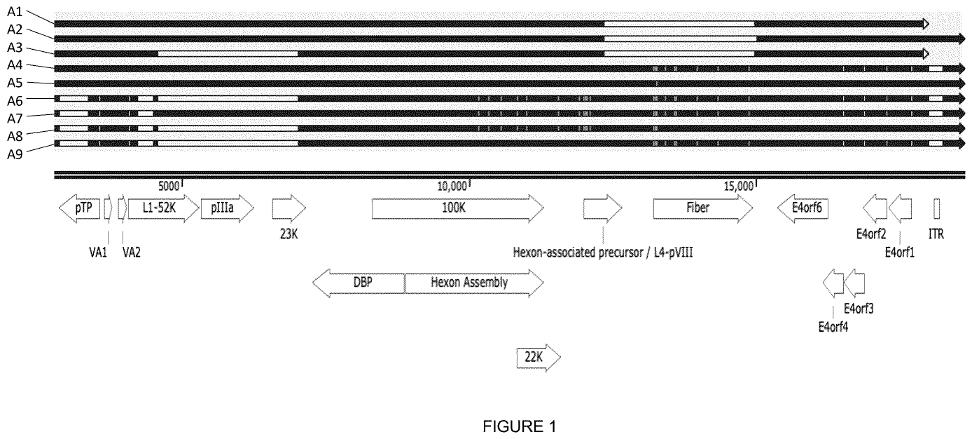

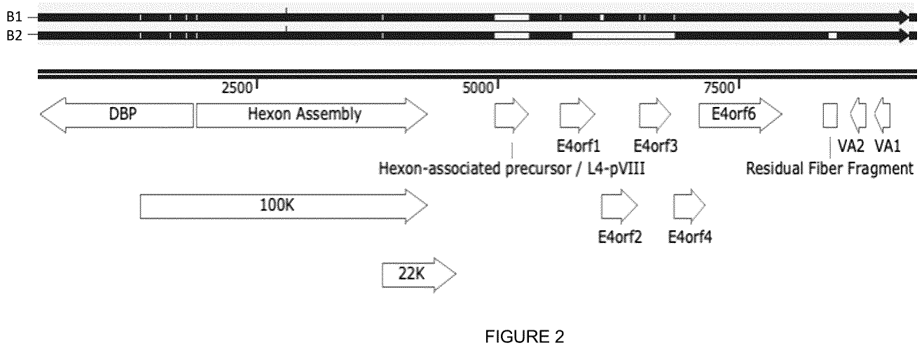

Provided below are non-limiting examples of specific AAV helper plasmid variants that find use in the present disclosure. The alignment of each plasmid variant to its parent helper plasmid is shown in FIG. 1 (for parent A variants) or FIG. 2 (for parent B variants). Each of the inactivating mutations listed below is described in detail in Table 2. The inactivating mutations in each variant helper plasmid (i.e., A1 to A9, B1 and B2) are listed in Table 3 along with the SEQ ID NO of each variant.

An example of an AAV helper plasmid of the disclosure is variant A4, which comprises (i) an inactivated adenoviral fiber gene comprising the following inactivating mutations: c.1_2AT>TA, c.22G>T, c.45T>A, c.196_199ATGG>TAG, c.361-362AT>TA, c.385-386AT>TA, c.751-752AT>TA, and c.1118delT; (ii) an inactivated adenoviral E4 region orf comprising the following inactivating mutations: E4orf1 c.1_2TA>AT, E4orf2 c.1T>A, E4orf3 c.1_2TA>AT, and E4orf4 c.1_2TA>AT; and (iii) an inactivating mutation of the ITR region (ITRDeletion1).

Another example of an AAV helper plasmid of the disclosure is variant A5, which comprises an inactivated adenoviral fiber gene comprising the following inactivating mutation: c.49delAT.

Another example of an AAV helper plasmid of the disclosure is variant A6, which comprises (i) an inactivated adenoviral fiber gene and comprising the following inactivating mutations: c.1_2AT>TA, c.22G>T, c.45T>A, c.196_199ATGG>TAG, c.361-362AT>TA, c.385-386AT>TA, c.751-752AT>TA, c.1118delT, and c.1654-1655AT>TA; (ii) an inactivated adenoviral precursor terminal protein (pTP) gene comprising the following inactivating mutations: pTP Deletion 1 and c.1T>A; (iii) an inactivated adenoviral L1-52K gene comprising the following inactivating mutations: c.3G>A, L1-52K Deletion 1, and L1-52K Deletion 2; (iv) an inactivated adenoviral 100K protein gene comprising the following inactivating mutations: c.1847T>A, c.2026A>T, c.2239_2240AT>TA, c.2519_2520AT>TA, and c.2685T>A; (v) an inactivated adenoviral PVIII protein gene comprising the following inactivating mutations: c.1T>A, c.25_29TACAT>ATCTA, c.49_50TA>AT, and c.100_101AT>TA; (vi) an inactivated adenoviral E4 region orf comprising the following inactivating mutations: E4orf1 c.1_2TA>AT, E4orf2 c.1T>A, E4orf3 c.1_2TA>AT, E4orf4 c.1_2TA>AT; and (vii) an inactivating mutation of the ITR region (ITRDeletion1).

Another example of an AAV helper plasmid of the disclosure is variant A7, which comprises (i) an inactivated adenoviral fiber gene comprising the following inactivating mutations: c.1_2AT>TA, c.22G>T, c.45T>A, c.196_199ATGG>TAG, c.361-362AT>TA, c.385-386AT>TA, c.751-752AT>TA, c.1118delT, and c.1654-1655AT>TA; (ii) an inactivated adenoviral pTP gene comprising the following inactivating mutations: pTP Deletion 1 and c.1T>A; (iii) an inactivated adenoviral L1-52K gene comprising the following inactivating mutations: c.3G>A and L1-52K Deletion 1; (iv) an inactivated adenoviral 100K gene comprising the following inactivating mutations: c.1847T>A, c.2026A>T, c.2239_2240AT>TA, c.2519_2520AT>TA, and c.2685T>A; (v) an inactivated adenoviral PVIII gene comprising the following inactivating mutations: c.1T>A, c.25_29TACAT>ATCTA, c.49_50TA>AT, and c.100_101AT>TA; (vi) an inactivated adenoviral E4 region orf comprises the following inactivating mutations: E4orf1 c.1_2TA>AT, E4orf2 c.1T>A, E4orf3 c.1_2TA>AT, E4orf4 c.1_2TA>AT; and (vii) an inactivating mutation of the ITR region (ITRDeletion1).

Another example of an AAV helper plasmid of the disclosure is variant A8, which comprises (i) an inactivated adenoviral fiber gene comprising the following inactivating mutations: c.1_2AT>TA, c.22G>T, and c.45T>A; (ii) an inactivated adenoviral pTP gene comprising the following inactivating mutations: pTP Deletion 1 and c.1T>A; (iii) an inactivated adenoviral L1-52K gene comprising the following inactivation mutations: c.3G>A, L1-52K Deletion 1, and L1-52K Deletion 2; (iv) an inactivated adenoviral 100K gene comprising the following inactivating mutations: c.1847T>A, c.2026A>T, c.2239_2240AT>TA, c.2519_2520AT>TA, and c.2685T>A; and (v) an inactivated adenoviral PVIII gene comprising the following inactivating mutations: c.1T>A, c.25_29TACAT>ATCTA, c.49_50TA>AT, and c.100_101AT>TA.

Another example of an AAV helper plasmid of the disclosure is variant A9, which comprises (i) an inactivated adenoviral fiber gene comprising the following inactivating mutations: c.1_2AT>TA, c.22G>T, c.45T>A, c.196_199ATGG>TAG, c.361-362AT>TA, c.386-386AT>TA, c.751-752AT>TA, c.1118delT, and c.1654-1655AT>TA; (ii) an inactivated adenoviral precursor terminal protein gene comprising the following inactivating mutations: pTP Deletion 1 and c.1T>A; (iii) an inactivated adenoviral L1-52K gene comprising the following inactivating mutations: c.3G>A, L1-52K Deletion 1, and L1-52K Deletion 2; (iv) an inactivated adenoviral E4 region orf comprising the following inactivating mutations: E4orf1 c.1_2TA>AT, E4orf2 c.1T>A, E4orf3 c.1_2TA>AT, E4orf4 c.1_2TA>AT; and (v) an inactivating mutation of the ITR region (ITRDeletion1).

Another example of an AAV helper plasmid of the disclosure is variant B1, which comprises (i) an inactivated adenoviral 100K gene comprising the following inactivating mutations: c.3G>T, c.312G>C, and c.480G>T; (ii) an inactivated adenoviral PVIII gene and comprising the inactivation mutation pVIII Deletion 1; (iii) an inactivated adenoviral E4 region orf comprising the following inactivating mutations: E4orf1 c.1_3delATG, E4orf2 c.-18_-16delATG (upstream), E4orf2 c.1_3delATG, E4orf2 c.16_18delATG, E4orf3 c.1_3delATG, E4orf3 c.55_57delATG, and E4orf4 c.1_3delATG; and (iv) an inactivated hexon assembly gene comprising the inactivating mutations c.1_3delATG, c.933insTAATAA, and c.1937-1938delAT.

Another example of an AAV helper plasmid of the disclosure is variant B2, which comprises (i) deletion of residual Fiber sequences (Fiber deletion 3); (ii) an inactivated adenoviral 100K gene comprising the following inactivating mutations: c.3G>T, c.312G>C, and c.480G>T; (iii) an inactivated adenoviral PVIII gene comprising the activating mutation pVIII Deletion 1; (iv) an inactivated adenoviral E4 region orf comprising the inactivating mutation E4 deletion 1; and (v) an inactivated hexon assembly gene comprising the inactivating mutations c.1_3delATG, c.933insTAATAA, and c.1937-1938delAT.

Aspects of the present disclosure include AAV helper plasmids that include the inactivating mutations present in variant A1 as listed in Table 3. In certain embodiments, these AAV helper plasmids have from 80% to 100% sequence identity to SEQ ID NO: 37, including at least 80%, at least 85%, at least 90%, at least 95%, at least 97%, at least 98%, at least 99%, and up to 100% sequence identity to SEQ ID NO: 37.

Aspects of the present disclosure include AAV helper plasmids that include the inactivating mutations present in variant A2 as listed in Table 3. In certain embodiments, these AAV helper plasmids have from 80% to 100% sequence identity to SEQ ID NO: 38, including at least 80%, at least 85%, at least 90%, at least 95%, at least 97%, at least 98%, at least 99%, and up to 100% sequence identity to SEQ ID NO: 38.

Aspects of the present disclosure include AAV helper plasmids that include the inactivating mutations present in variant A3 as listed in Table 3. In certain embodiments, these AAV helper plasmids have from 80% to 100% sequence identity to SEQ ID NO: 39, including at least 80%, at least 85%, at least 90%, at least 95%, at least 97%, at least 98%, at least 99%, and up to 100% sequence identity to SEQ ID NO: 39.

Aspects of the present disclosure include AAV helper plasmids that include the inactivating mutations present in variant A4 as listed in Table 3. In certain embodiments, these AAV helper plasmids have from 80% to 100% sequence identity to SEQ ID NO: 40, including at least 80%, at least 85%, at least 90%, at least 95%, at least 97%, at least 98%, at least 99%, and up to 100% sequence identity to SEQ ID NO: 40.

Aspects of the present disclosure include AAV helper plasmids that include the inactivating mutations present in variant A5 as listed in Table 3. In certain embodiments, these AAV helper plasmids have from 80% to 100% sequence identity to SEQ ID NO: 41, including at least 80%, at least 85%, at least 90%, at least 95%, at least 97%, at least 98%, at least 99%, and up to 100% sequence identity to SEQ ID NO: 41.

Aspects of the present disclosure include AAV helper plasmids that include the inactivating mutations present in variant A6 as listed in Table 3. In certain embodiments, these AAV helper plasmids have from 80% to 100% sequence identity to SEQ ID NO: 42, including at least 80%, at least 85%, at least 90%, at least 95%, at least 97%, at least 98%, at least 99%, and up to 100% sequence identity to SEQ ID NO: 42.

Aspects of the present disclosure include AAV helper plasmids that include the inactivating mutations present in variant A7 as listed in Table 3. In certain embodiments, these AAV helper plasmids have from 80% to 100% sequence identity to SEQ ID NO: 43, including at least 80%, at least 85%, at least 90%, at least 95%, at least 97%, at least 98%, at least 99%, and up to 100% sequence identity to SEQ ID NO: 43.

Aspects of the present disclosure include AAV helper plasmids that include the inactivating mutations present in variant A8 as listed in Table 3. In certain embodiments, these AAV helper plasmids have from 80% to 100% sequence identity to SEQ ID NO: 44, including at least 80%, at least 85%, at least 90%, at least 95%, at least 97%, at least 98%, at least 99%, and up to 100% sequence identity to SEQ ID NO: 44.

Aspects of the present disclosure include AAV helper plasmids that include the inactivating mutations present in variant A9 as listed in Table 3. In certain embodiments, these AAV helper plasmids have from 80% to 100% sequence identity to SEQ ID NO: 45, including at least 80%, at least 85%, at least 90%, at least 95%, at least 97%, at least 98%, at least 99%, and up to 100% sequence identity to SEQ ID NO: 45.

Aspects of the present disclosure include AAV helper plasmids that include the inactivating mutations present in variant B1 as listed in Table 3. In certain embodiments, these AAV helper plasmids have from 80% to 100% sequence identity to SEQ ID NO: 46, including at least 80%, at least 85%, at least 90%, at least 95%, at least 97%, at least 98%, at least 99%, and up to 100% sequence identity to SEQ ID NO: 46.

Aspects of the present disclosure include AAV helper plasmids that include the inactivating mutations present in variant B2 as listed in Table 3. In certain embodiments, these AAV helper plasmids have from 80% to 100% sequence identity to SEQ ID NO: 47, including at least 80%, at least 85%, at least 90%, at least 95%, at least 97%, at least 98%, at least 99%, and up to 100% sequence identity to SEQ ID NO: 47.

The disclosure also provides a plasmid system comprising (i) an AAV helper plasmid variant described herein and (ii)(a) a plasmid comprising a nucleic acid encoding an AAV Rep protein and a nucleic acid encoding an AAV Cap protein or (ii)(b) a plasmid comprising a nucleic acid encoding an AAV Rep protein and a plasmid comprising nucleic acid encoding an AAV Cap protein. Optionally, the system further comprises (iii) a plasmid comprising at least one heterologous nucleic acid, wherein the heterologous nucleic acid(s) is flanked by a 5′ and 3′ AAV inverted terminal repeat (ITR). The plasmid system may be provided with each of the different types of plasmid present in a single vessel, or may be provided as a kit wherein each of the different types of plasmid is provided in separate vessels. For example, the system may be provided such that plasmid (i) and plasmid(s) (ii) are provided in the same vessel or the same cell, or the plasmids may be provided in separate vessels or cells. When plasmid (iii) is present, it may be combined with either or both of plasmid (i) or plasmid(s) (ii) in the same vessel or cell, or may be provided separately.

The system of the disclosure comprises one or more plasmids which encode(s) proteins that supply the functions of the AAV Rep and Cap genes. In this regard, the system comprises (a) a plasmid comprising a nucleic acid encoding one or more AAV Rep proteins and a nucleic acid encoding one or more AAV Cap proteins or (b) a plasmid comprising a nucleic acid encoding one or more AAV Rep proteins and a plasmid comprising a nucleic acid encoding one or more AAV Cap proteins. Four AAV Rep proteins are encoded within the single open reading frame present in the AAV genome ad are produced by alternative RNA splicing: Rep78, Rep68, Rep52, and Rep40. The Rep proteins have a variety of functions including, but not limited to, DNA helicase activity, endonuclease activity, promoter modulation activity, and mediation of viral assembly. In various aspects of the disclosure, Rep-encoding nucleic acid(s) encodes the Rep78 protein and the Rep52 and/or Rep40 proteins; the Rep68 and the Rep52 and/or Rep40 proteins; the Rep68 and Rep52 proteins; the Rep68 and Rep40 proteins; the Rep78 and Rep52 proteins; the Rep78 and Rep40 proteins; or the Rep78, Rep68, Rep52 and Rep40 proteins. The Rep-encoding nucleic acid provides the functions required for AAV virion production. The AAV Cap gene encodes multiple structural proteins required for viral packaging, called VP1, VP2, and VP3. Typically, the Cap-encoding sequence will encode all of the AAV capsid subunits, but less than all of the capsid subunits may be encoded as long as a functional capsid is produced (VP1, VP2, and/or VP3). The Rep- and/or Cap-encoding sequences can be derived from any suitable AAV serotype, including serotypes AAV1, AAV2, AAV3, AAV4, AAV5, AAV6, AAV7, AAV8, AAV9, AAV10, and/or AAV11, or a chimera thereof. If desired, the Rep- and/or Cap-encoding sequences may be selected based on the tropism of the parent AAV virus, and the serotype may be different than the serotype of the AAV ITRs used in the payload plasmid. The Rep- and/or Cap-encoding sequences may also be selected to provide a hybrid capsid comprising elements from different serotypes. Examples of Rep/Cap helper plasmids are disclosed in, e.g., Samulski et al. (1989) J. Virol. 63:3822-3828; McCarty et al. (1991) J. Virol. 65:2936-2945; and U.S. Pat. Nos. 5,139,941; 6,001,650; 6,376,237; and 7,259,151, each of which is incorporated herein by reference in their entirety and particularly with respect to disclosures relating to supply of Rep and Cap proteins in trans for AAV virus production.

The materials and methods described herein are useful for, e.g., the manufacture of recombinant adeno-associated virus (AAV) vectors comprising a heterologous nucleic acid. Indeed, the system of the disclosure comprises a plasmid comprising at least one heterologous nucleic acid flanked by a 5′ and 3 AAV inverted terminal repeat (ITR) (also referred to herein as a “payload plasmid”), which is packaged into AAV virions suitable for gene therapy applications. It will be appreciated that aspects of the disclosure herein regarding AAV vectors also applies to the payload plasmid of the system. The term “AAV” includes all serotypes of AAV, including AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8, and hybrids thereof (i.e., chimeric AAV vectors). The genomic sequences of various serotypes of AAV, as well as the sequences of the native terminal repeats (TRs), Rep proteins, and capsid subunits are known in the art. Such sequences may be found in the literature or in public databases such as GenBank. By “heterologous nucleic acid” is meant a polynucleotide sequence not of AAV origin, typically a sequence of interest for delivery to a host cell. In general, the heterologous polynucleotide is flanked by at least one, and generally by two, AAV inverted terminal repeat sequences (ITRs). An AAV vector may either be single- stranded (ssAAV) or self-complementary (scAAV). See, e.g., Raj et al., Expert Rev Hematol. 2011 October; 4(5): 539-549. AAVs may comprise genome components and capsids from multiple serotypes (e.g., pseudotyped vectors). The payload plasmid or recombinant AAV vector comprises inverted terminal repeats (ITRs), which may be derived from the same serotype as the capsid of the virus particle or derived from a different serotype (e.g., AAV2 ITRs and AAV9 capsid proteins; AAV2 ITRs and AAV8 capsid proteins; AAV2 ITRs and AAV5 capsid proteins; etc.). In a representative embodiment, the recombinant AAV vector comprises AAV2 ITRs. Pseudotyped vectors may demonstrate improved transduction efficiency as well as altered tropism. In some cases, an AAV serotype that can cross the blood brain barrier or infect cells of the CNS is preferred. In some aspects, the recombinant AAV vector is AAV1, AAV8, AAV9, AAVDJ, or chimeric AAV comprising features of two or more of these serotypes. In various embodiments, the AAV vector is an AAV9 vector or a scAAV9 vector.

The payload plasmid or resulting recombinant AAV vector comprises a heterologous nucleic acid. The heterologous nucleic acid may comprise, or be in the form, of an “expression cassette,” referring to a polynucleotide comprising one or more regulatory elements operably linked to a coding sequence (i.e., a polynucleotide sequence encoding an RNA or peptide of interest). The coding sequence is sometimes referred to herein as a transgene. The recombinant AAV vector may comprise any heterologous nucleic acid of interest, including a heterologous nucleic acid that encodes a functional nucleic acid or peptide/polypeptide of interest. For example, the heterologous nucleic acid may encode an agonist, an antagonist, an antigen, an anti-apoptosis factor (i.e., an apoptosis inhibitor), an angiogenic factor, an anti-angiogenic factor, an anti-viral factor, an anti-bacterial factor, an anti-fungal factor, a receptor, a blood factor (e.g., blood factor VII, blood factor VIIa, blood factor VIII, blood factor IX, blood factor XIII, etc.), a cytokine or cytokine receptor, a chemokine or chemokine receptor, a cytotoxin, an erythropoietic agent, a glycoprotein, a growth factor (e.g., Nerve Growth Factor, Ciliary Neurotrophic Factor, Insulin-like Growth Factor, Myelopoiesis Growth Factor, Epithelial Growth Factor, Epidermal Growth Factor, Glioma Derived Growth Factor (GDGF), Platelet Derived Growth Factor-A (PDGF-A), Platelet Derived Growth Factor-B (PDGF-B), Placental Growth Factor (PIGF), Placental Growth Factor-2 (PIGF-2), Vascular Endothelial Growth Factor (VEGF) (e.g., Vascular Endothelial Growth Factor-A (VEGF-A), Vascular Endothelial Growth Factor-2 (VEGF-2), Vascular Endothelial Growth Factor B (VEGF-3), Vascular Endothelial Growth Factor B-186 (VEGF-B186), Vascular Endothelial Growth Factor-D (VEGF-D), Vascular Endothelial Growth Factor-D (VEGF-D), or Vascular Endothelial Growth Factor-E (VEGF-E)), a fibroblast growth factor (such as FGF-1, FGF-2, FGF-3, FGF-4, FGF-5, FGF-6, FGF-7, FGF-8, FGF-9, FGF-10, FGF-11, FGF-12, FGF-13, FGF-14, or FGF-15), a hematopoietic growth factor (e.g., granulocyte macrophage colony stimulating factor (GM-CSF), granulocyte colony stimulating factor (G-CSF) (filgrastim), macrophage colony stimulating factor (M-CSF, CSF-1) erythropoietin (epoetin alfa), stem cell factor (SCF, c-kit ligand, steel factor), megakaryocyte colony stimulating factor, etc.), a growth factor receptor, a hormone or hormone receptor (e.g., growth hormone, growth hormone release hormones, follicle stimulating hormone, progesterone forming hormone, progesterone forming hormone releasing hormone, thyroid stimulating hormone, etc.), an interferon or interferon receptor (e.g., interferon-alpha, -beta and -gamma, Type I soluble interferon receptor, etc.), an interleukin or interleukin receptor (e.g., IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, or IL-21), an immuno-costimulatory factor, a neuroactive peptide or neuroactive peptide receptor, a neurogenic factor or neurogenic factor receptor, a neurotrophic factor, a neurotransmitter regulator, a nuclease (e.g., a CRISPR/Cas9 nuclease), a protease, a protease inhibitor, a protein decarboxylase, a protein kinase, a protein kinase inhibitor, an enzyme, a receptor binding protein, a transport protein or inhibitor thereof, an ion channel or component thereof or inhibitor thereof, a serotonin receptor or an uptake inhibitor thereof, a serpin, a serpin receptor, a tumor suppressor, a tumor necrosis factor (e.g., TNF-alpha, TNF-beta, TNF-gamma), a receptor antagonist (e.g., IL1-Ra, etc.), a cell surface antigen (e.g., CD 2, 3, 4, 5, 7, 11a, 11b, 18, 19, 20, 23, 25, 33, 38, 40, 45, 69, etc.), a transcription factor, an antibody or fragment thereof, an antibody-like protein which binds a target (epitope) (e.g., a single chain antibody, nanobody, and the like), a vasoactive agent, or any combination thereof (optionally presented as a fusion protein). The heterologous nucleic acid may also encode a functional nucleic acid, such as a ribozyme, siRNA, RNAi, miRNA, an antisense oligonucleotide, and the like.

In some instances, the payload plasmid or recombinant AAV vector comprises a heterologous nucleic acid that encodes an ion channel, a neurotransmitter regulator, a transcription factor, or a subunit, variant, or functional fragment of any of the foregoing. Examples of ion channels include voltage gated and ligand gated ion channels. Voltage gated ion channels include sodium channels, calcium channels, potassium channels, and proton channels. In some embodiments, the transgene encodes SCN1A, SCN2A, SCN8A, SCN1B, SCN2B, KV3.1, KV3.2, KV3.3, STXBP1, KCNC1, KCNC3, or isoforms, variants, or functional fragments thereof. For example, the payload plasmid or resulting recombinant AAV vector comprises a transgene encoding a polypeptide comprising an amino acid sequence having at least 80%, at least 85%, at least 90%, at least 95%, at least 96%, at least 97%, at least 98%, or at least 99% sequence identity to the amino sequence of any one of SCN1A, SCN2A, SCN8A, SCN1B, SCN2B, KV3.1, KV3.2, KV3.3, STXBP1, KCNC1, KCNC3, or a functional fragment thereof.

Another example of a heterologous nucleic acid of interest encodes a transcription factor, including non-naturally occurring (engineered) transcription factors, which may be a transcription activator or a transcription repressor. A transcription factor comprises a DNA binding domain (DBD) and a transcription modulation domain (TMD). A DNA binding domain binds a target site in DNA. A TMD contains binding sites for other proteins that promote or repress transcription of a target gene/nucleic acid sequence. The TMD may contact transcriptional machinery (e.g., RNA polymerase) either directly or through other proteins (known as co-activators or co-modulators). The transcription factor may be wildtype (i.e., unmodified) or may be a non-naturally occurring transcription factor, such as a transcription factor comprising an engineered DBD, a DBD operably linked to a TMD to which it is not naturally linked (e.g., derived from a different transcription factor or from a different species). In various aspects, the heterologous nucleic acid encodes a transcription factor that modulates expression (e.g., enhances expression) of one or more of SCN1A, SCN2A, SCN8A, SCN1B, SCN2B, KV3.1, KV3.2, KV3.3, STXBP1, KCNC1, or KCNC3.

Examples of DBDs include zinc fingers, helix-turn-helix, leucine zipper (e.g., bZIP), helix-loop-helix, and beta-scaffold Cas9, a Cas family protein, dCas9, a dCas family protein, or a transcriptional activator like effector (TALE). In some cases, the recombinant AAV vector (or payload plasmid) comprises a transgene that encodes a DNA binding protein comprising a DNA cleaving region that has been deactivated. In some cases, the transgene comprises a gene editing protein, e.g., a Cas protein, Cas9. The heterologous nucleic acid may encode multiple copies of the same DNA binding domain, or may comprise multiple DNA binding domains of different sequences. For example, various aspects of the disclosure provide recombinant AAV vector (or payload plasmid) comprising a heterologous nucleic acid comprising from 2 to 10 DNA binding domains, such as zinc fingers (e.g., 3 to 8 zinc fingers, or 1, 2, 3, 4, 5, 6, 7, 8, 9, or 10 zinc fingers).

Examples of suitable DBDs are described in International Patent Publication Nos. WO 2019/109051 (entitled “Engineered DNA Binding Proteins”) and WO 2020/243651 (entitled “Compositions and Methods for Selective Gene Regulation”), each of which is incorporated herein by reference in its entirety.

The TMD(s) and DNA binding domain(s) (DBD) may be derived from different proteins. An engineered TF may comprise more than one TMD, and two or more of the TMDs may be derived (e.g., isolated from) different proteins compared to other TMD(s) in the protein. In various aspects, the TMD is a transactivation domain, which enhances or upregulates expression. Examples of transactivation domains include those derived from known transcription activation proteins, e.g., VP64, VPR, VP16, VP128, p65, p300, CBP/p300-interacting transactivator 2 (CITED2), CBP/p300-interacting transactivator 4 (CITED4), EGR1, or EGR3. Any suitable arrangement of one or more DBDs and one or more TMDs is contemplated.

Native transcription factors may be promiscuous, meaning they are active in most cell types. Transcription factors also may be a tissue-specific, such as those from muscle cells (e.g., MyoD and muscle enhancer factor 2 (MEF2)) or those from neuronal cells (e.g., nuclear factor 1C (NF1C), nuclear factor 1X (NF1X), Brain-1 (Brn-1), or Brain-2 (Brn-2)). Transcription factors also may be ligand-dependent. Ligand-dependent transcription factors comprise an additional domain which is bound by the ligand, which results in up-or down-regulation of gene expression. Steroid hormone receptors and nuclear receptors are examples of ligand-dependent transcription factors. Other examples of ligand-dependent transcription factors are metal-responsive transcription factors that, e.g., regulate metal (iron, zinc, or copper) homeostasis.

Examples of transcription factors include, but are not limited to, AF-4 transcription factors, Androgen receptor transcription factors, AP-2 transcription factors, ARID transcription factors, bHLH transcription factors, C/EBP transcription factors, CBF transcription factors, CG-1 transcription factors, COE transcription factors, COUP transcription factors, CP2 transcription factors, CSD transcription factors, CSL transcription factors, CTF/NFI transcription factors, CUT transcription factors, DM transcription factors, E2F transcription factors, EAF2 transcription factors, Ecdystd receptor transcription factors, ETS transcription factors, Fork head transcription factors, GCM transcription factors, GCR transcription factors, GTF2I transcription factors, HMG transcription factors, HMGI/HMGY transcription factors, Homeobox transcription factors, HSF transcription factors, HTH transcription factors, IRF transcription factors, MBD transcription factors, MH1 transcription factors, MYB transcription factors, NDT80/PhoG transcription factors, NF-YA transcription factors, NF-YB/C transcription factors, Nrf1 transcription factors, Nuclear orphan receptor transcription factors, Oestrogen receptor transcription factors, P53 transcription factors, PAX transcription factors, PC4 transcription factors, POU transcription factors, PPAR receptor transcription factors, PREB transcription factors, Progesterone receptor transcription factors, Prox1 transcription factors, Retinoic acid receptor transcription factors, RFX transcription factors, RHD transcription factors, ROR receptor transcription factors, Runt transcription factors, SAND transcription factors, SPZ1 transcription factors, SRF transcription factors, STAT transcription factors, T-box transcription factors, TEA transcription factors, TF-bZIP transcription factors, TF-Otx transcription factors, THAP transcription factors, Thyroid hormone receptor transcription factors, TSC22 transcription factors, Tub transcription factors, ZBTB transcription factors, zf-BED transcription factors, zf-C2H2 transcription factors, zf-C2HC transcription factors, zf-GATA transcription factors, zf-LITAF-like transcription factors, zf-MIZ transcription factors, and zf-NF-X1 transcription factors. Metal-responsive transcription factors include, but are not limited to, Aft1, Aft2, Fep1, SREA, Urbs1, Ace1, Amt1, Srf1, Mac1, Cuf1, GRISEA, Crr1, Zap1, and metal response element-binding transcription factor-1 (MTF-1). MTF-1 induces expression of metallothioneins and other genes involved in metal homeostasis in response to heavy metals such as copper. See, e.g., Rutherford and Bird, Eukaryot Cell. 2004 February; 3(1): 1-13; and Wang et al., Biol Chem. 2004 July;385(7):623-32.

The transcription factor may be any of the transcription factors disclosed herein, or may comprise components of any of the referenced transcription factors referenced herein (e.g., the DBD or the TMD of the referenced transcription factors). In exemplary aspects of the disclosure, the heterologous nucleic acid encodes a transcription factor that upregulates SCN1A production and is any of the engineered transcription factors described in International Patent Publication No. WO 2020/243651, incorporated herein by reference in its entirety. .

The heterologous nucleic acid sequence optionally comprises a promoter to drive expression of the nucleic acid. A promoter can be native or non-native to the nucleic acid sequence to which it is operably linked, and native or non-native to a particular host cell. A promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Additional examples of constitutive promoters include, a GAD2 promoter, a human synapsin promoter, CBA promoter, a minCMV promoter, a TATA box, a super core promoter, or an EF1a promoter. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. In various embodiments, the heterologous nucleic acid comprises the CMV promoter.

Optionally, the heterologous nucleic acid comprises one or more additional regulatory elements (optionally in addition to a promoter), such as, for example, sequences associated with transcription initiation or termination, enhancer sequences, and efficient RNA processing signals. Exemplary regulatory elements include, for example, an intron, an enhancer, UTR, stability element, WPRE sequence, a Kozak consensus sequence, posttranslational response element, a microRNA binding site, a polyadenylation (polyA) signal sequence, or a combination thereof. Regulatory elements can function to modulate gene expression at the transcriptional phase, post-transcriptional phase, or at the translational phase of gene expression. At the RNA level, regulation can occur at the level of translation (e.g., stability elements that stabilize mRNA for translation), RNA cleavage, RNA splicing, and/or transcriptional termination.

Regulatory elements included in the heterologous nucleic acid may be cell type selective regulatory elements, such as regulatory elements that drive expression in central nervous system cell types. Optionally, the regulatory element(s) selectively drive expression in GABAergic cells. GABAergic cells are inhibitory neurons which produce gamma-aminobutyric acid. GABAergic cells can be identified by the expression of glutamic acid decarboxylase 2 (GAD2). Other markers of GABAergic cells include GAD1, NKX2.1, DLX1, DLX5, SST, PV and VIP. The regulatory element(s) may selectively drive expression in GABAergic cells that express parvalbumin (“PV cells”), to a greater degree than another cell type (e.g., another CNS cell type, such as a non-GABAergic neuron (e.g., non-PV GABAergic neurons)). Examples of non-PV CNS cells include excitatory neurons, dopaminergic neurons, astrocytes, microglia, motor neurons, and vascular cells. Non-GABAergic neurons also include cells that do not express one or more of GAD2, GAD1, NKX2.1, DLX1, DLX5, SST and VIP. In some cases, non-PV GABAergic neurons include, but are not limited to, calretinin (CR), somatostatin (SOM), cholecystokinin (CCK), CR+SOM, CR+neuropeptide Y (NPY), CR+vasointestinal polypeptide (VIP), SOM+NPY, SOM+VIP, VIP+choline acetyltransferase (ChAT), CCK+NPY, CR+SOM+NPY, and CR+SOM+VIP expressing cells.

In some aspects, the payload plasmid or recombinant AAV vector comprises a PV-selective regulatory element as described in International Patent Publication No. WO 2018/187363 (entitled “Tissue Selective Transgene Expression”), incorporated herein by reference in its entirety.

In certain embodiments, the heterologous nucleic acid further comprises a polyA signal sequence.

Suitable polyA signal sequences include, for example, an artificial polyA that is about 75 bp in length (PA75) (see e.g., International Patent Publication No. WO 2018/126116), the bovine growth hormone polyA, SV40 early polyA signal, SV40 late polyA signal, rabbit beta globin polyA, HSV thymidine kinase polyA, protamine gene polyA, adenovirus 5 Elb polyA, growth hormone polyA, or a PBGD polyA. In exemplary embodiments, the polyA sequence is an hGH polyA or a synthetic polyA, e.g., as described in International Patent Publication No. WO 2019/109051 (entitled “Engineered DNA Binding Proteins”), incorporated herein by reference in its entirety.

Typically, the polyA signal sequence is operably linked to a coding nucleic acid sequence.

The disclosure further provides a host cell comprising the AAV helper plasmid or system described herein. The host cell is able to support AAV genome replication and packaging (sometimes referred to as an “AAV packaging cell”). In various aspects, the host cell is a mammalian cell. Examples of host cells include, but are not limited to, Human Embryonic Kidney (HEK) 293 cells (e.g., HEK 293T cells), CHO cells, Jurkat cells, A549 cells, KS62 cells, PerC6 cells, KB cells, and HeLa cells. HEK 293 cells include adenoviral E1A and E1B genes incorporated into the cellular genome. It will be appreciated that derivatives of these parent cell lines also are contemplated in the context of the disclosure. Host cells may be adherent cells (i.e., cells which grow in a single layer attached to a surface) or suspension cells (i.e., cells which grow in suspension in a culture medium).

The plasmids described herein may be introduced into host cells using any suitable method. Methods of introducing heterologous nucleic acid into host cells include, but are not limited to, lipid-mediated transfection, microinjection, electroporation, microprojectile bombardment, or chemical-mediated transduction. For example, transduction may be facilitated using polyethylenimine (PEI) or other cationic polymers, chitin, or calcium phosphate. In various aspects, the host cell(s) comprising the plasmid(s) of the disclosure are cultured under conditions to allow AAV replication and packaging. Culture conditions suitable for AAV production are known in the art. Merely to illustrate, examples of culture conditions include, e.g., incubation at 37° C. at 5% CO₂. The host cell may be cultured in any medium suitable for the particular cell type. A suitable culture media is, for example, Dulbecco's modified Eagle's medium (DMEM) (optionally containing 10% (vol/vol) fetal bovine serum (FBS)).

The disclosure further provides a method of producing recombinant AAV, the method comprising culturing the host cell(s) described herein and isolating recombinant AAV. The disclosure further provides a recombinant AAV produced by the method described herein. The disclosure is not specific to a particular culture system, and may be utilized with tissue culture flasks, multiwell plates, roller bottles, multi-layer tissue flasks, agitated flasks, agitated bottles, microcarriers, fiber discs, rocking bioreactors, stirred-tank bioreactors, airlift bioreactors, orbital bioreactors, hollow-fiber bioreactors, or other suitable culture system. The culture may be operated in batch, fed-batch, semi-batch, chemostat, or perfusion modes, or any combination thereof. In various aspects, the host cells are lysed to release the recombinant AAV, allowing isolation of the AAV produced by the method. Cells may be lysed via chemical lysis, e.g., using a detergent, mechanical lysis, or a combination thereof. Methods of chemical and mechanical lysis are known in the art. Lysis solutions can include one or more buffering agents, solubilizing agents, surfactants, preservatives, cryoprotectants, enzymes, enzyme inhibitors and/or chelators. Mechanical cell lysis may be performed under any condition that promotes cellular disruption but does not significantly damage the resulting AAV stock, for example, certain temperatures, pressures, or osmotic purity. Representative mechanical lysis techniques include, but are not limited to, freeze-thaw lysis, use of mechanical forces (e.g., grinding or pressure), use of sonic forces, use of gravitational forces, use of electrical forces and the like. In some aspects, the lysis process involves cell centrifugation to clear the lysate and remove debris, and optionally comprises further purification steps. Nonlimiting examples of various materials and methods of AAV production are described in Clement and Grieger, “Manufacturing of recombinant adeno-associated viral vectors for clinical trials,” Mol. Ther. Methods Clin Dev. 3:16002 (2016) and Grieger et al., “Production of recombinant adeno-associated virus vectors using suspension HEK293 cells and continuous harvest of vector from the culture media for GMP FIX and FLT1 clinical vector,” Mol Ther 24(2):287-297 (2016), the contents of which are incorporated by reference herein.

An advantage of the materials and methods of the disclosure is efficient production of recombinant AAV while minimizing the potential for contaminants in the resulting viral vector stock, e.g., a drug substance for use in gene therapy in a mammalian subject, e.g., a human subject. In this regard, culturing the host cell using the materials and methods described herein optionally results in packaging of viral genome into viral capsids such that the capsid protein to viral genome ratio is 10:1 or less, e.g., a capsid protein to viral genome ratio of 6:1 or less, 5:1 or less, 4:1 or less, 3:1 or less, 2:1 or less, or 1:1. Robust packaging efficiency is one exemplary advantage of the materials and methods described herein. Methods of determining a capsid protein to viral genome ratio are known in the art, and an exemplary method is described in the Examples below. Robust production of AAV vectors is another exemplary advantage of the materials and methods described herein. In this regard, in various aspects, AAV production achieved using the materials and methods described herein may be within 80% of the production (e.g., within 85%, within 88%, within 90%, within 92%, within 94%, within 95%, within 96%, within 98% of the level of production) achieved using an AAV helper plasmid that does not comprise the one or more inactivated adenoviral genes (i.e., a plasmid which comprises a functional adenoviral gene(s) corresponding to the gene(s) that are inactivated in the AAV helper plasmid). Representative AAV helper plasmids comprising functional adenoviral genes for use for comparison in the context of the instant method include the nucleic acid sequences set forth in SEQ ID NO: 1 (referred herein as “Parent A”) and SEQ ID NO: 2 (referred to herein as “Parent B”). “AAV production” refers to the amount of vector produced, which may be characterized as, e.g., viral genomes per milliliter (vg/mL) or infectious units per milliliter (iu/mL). Methods of characterizing AAV vector production are known in the art, and a representative methodology is provided in the Examples below. A further advantage of the materials and methods of the disclosure is reduced levels of potential contaminating adenoviral material in the AAV composition produced as described herein. In various aspects, the host cell comprises lower levels of mRNA transcripts or proteins encoded by the inactivated genes compared to a host cell comprising an AAV helper plasmid that does not comprise the one or more inactivated adenoviral genes (e.g., Parent A helper plasmid (SEQ ID NO: 1) or Parent B helper plasmid (SEQ ID NO: 2), which comprise functional versions of the various inactivated genes described herein). For instance, the AAV helper plasmid produces at least 10% less, at least 20% less, at least 30% less, at least 40% less, at least 50% less, at least 60% less, at least 70% less, at least 80% less, or at least 90% less of an RNA transcript or protein encoded by the Ad gene compared to, e.g., Parent A or Parent B, or does not produce a detectable amount of the RNA transcript or protein. Methods of characterizing RNA transcripts or peptides produced by a host cell are known in the art, and a representative methodology is provided in the Examples below. A decrease in the production of unwanted adenoviral proteins results in reduced amounts of these byproducts in the AAV culture, reducing the burden in purifying a stock to prepare an AAV pharmaceutical composition. In addition, in the event that off-target packaging of adenoviral sequences into AAV capsids may occur, the resulting AAV contaminant is not able to efficiently produce functional adenoviral proteins in a patient receiving the AAV therapy, thereby minimizing potential toxic side effects and/or unwanted immune responses.

### EXAMPLES

As numerous gene therapy products based on AAV vectors move towards clinical use, there is continual need to improve the safety of these therapeutics. One strategy to improve AAV vector safety is to inactivate adenoviral genes used in the production process that are not essential to the formation of functional AAV vectors. The Examples provided in this section describe results from our characterization of AAV vector production using AAV helper plasmid variants in which one or more adenoviral genes are inactivated. The data described below demonstrate that AAV helper plasmids comprising one or more inactivated adenoviral genes mediate efficient production of AAV thereby improving the safety profile of the resulting product.

### Example 1: Description of Methods and AAV Helper Plasmid Variants

Shake flask: To generate rAAV9, HEK293 cells containing a stable integration of the Ad5 E1 sequence were cultured in shake flask suspension cultures (30 mL) and transiently transfected using polyethyleneimine (PEI) with plasmids containing 1) AAV2 Rep gene sequence and AAV9 Cap gene sequence, 2) a nucleotide of interest flanked by ITRs (GOI plasmid), and 3) adenovirus gene-containing helper AAV plasmid. Cells were harvested and lysed to release rAAV9, and crude lysate was used for downstream analysis to quantify vector genome titer and AAV capsid abundance (capsid ELISA). The ratio of vector genome titer to capsid protein titer was used to generate an estimate of empty:full AAV particles (non-genome containing: AAV genome-containing capsids).

Bioreactor methods: To generate rAAV9, HEK293 cells containing a stable integration of the Ad5 E1 sequence were cultured in 2.0 L bioreactor suspension cultures and transiently transfected using polyethyleneimine (PEI) with plasmids containing 1) AAV2 Rep gene sequence and AAV9 Cap gene sequence, 2) a nucleotide of interest flanked by ITRs (GOI plasmid), and 3) adenovirus gene-containing helper AAV plasmid. Cells were harvested and lysed to release rAAV9, and crude lysate samples were collected. Cell lysate was further filtered to generate a clarified harvest, and samples from each were used for downstream analysis to quantify vector genome titer, and AAV capsid abundance (capsid ELISA). The ratio of vector genome titer to capsid protein titer was used to generate an estimate of empty:full AAV particles (non-genome containing: AAV genome-containing capsids).

Fiber transcript expression (qRT-PCR): Adherent HEK293 cells were plated in a 20-well plate (4.2×10⁵ cells per well) and transiently transfected with individual helper plasmids 24h post plating. Cells were harvested 24 hours post-transfection. RNA was extracted, and mRNA was reverse transcribed (Superscript IV RT system, Invitrogen) using OligoDT primers. qRT-PCR (Taqman FastAdvance Master Mix) was performed on cDNA in accordance with manufacturer's recommendations using the primer probe sets in Table 1. The fiber (5′) primer set targets a region at the 5′ end of the fiber mRNA transcript while the fiber (3′) primer set targets a region at the 3′ end of the fiber mRNA transcript. Relative fiber mRNA expression was determined using the delta-delta Ct method with respect to a control gene open reading frame (E4orf6) commonly expressed by all helper plasmids.

TABLE 1

| Primer | Forward (5′-3′) | Reverse (5′-3′) | Probe |
|---|---|---|---|
| Fiber | GAAGCGCGCAAGACCG | CGGATAGGCGCAAAGAGAG | ACGGAAACCGGTCCTCCAACTG |
| (5′) | (SEQ ID NO: 26) | (SEQ ID NO: 27) | TGCC (SEQ ID NO: 28) |
| Fiber | CGGAGACAAAACTAAAC | TTGTGGCCAGACCAGTCC | ACGGTACACAGGAAACAGGAGA |
| (3″) | CTGTAACAC (SEQ ID | (SEQ ID NO: 30) | CACAACTCC (SEQ ID NO: 31) |
| | NO: 29) | | |
| E4orf6 | AGCGCGCGAATAAACTG | TAAGTGAGATCAGGGTGCGC | CGCTCCGTCCTGCAGGAATACA |
| | C (SEQ ID NO: 32) | (SEQ ID NO: 33) | ACAT (SEQ ID NO: 34) |

Vg titer methods: AAV vector genome titer was determined by ddPCR (BioRad) using standard methodology. Briefly, lysate samples were diluted in buffer, DNAse-treated to remove extra-capsid DNA, and absolute quantification of vector genomes were determined in triplicate via ddPCR (BioRad) using primer:probe mix designed and validated against the AAV vector genome sequence.

Cp ELISA methods: Capsid protein abundance in harvest samples were quantified in duplicate by colorimetric sandwich ELISA using a modified from AAV9 Titration ELISA (PROGEN, Cat #PRAAV9). Briefly, a sandwich enzyme-linked immunosorbent assay (ELISA) was used to detect and quantify the level of AAV serotype 9 particles in samples. A monoclonal antibody specific for a conformational epitope on AAV9 capsids is pre-immobilized to the wells of strips on a microtiter plate. A biotin conjugated detection antibody that recognizes distinct epitope of AAV9 capsids was co-incubated with AAV9 particles in samples to form the immune complex. A streptavidin peroxidase conjugate was applied, then a TMB (tetramethylbenzidine) substrate solution was added producing a visible signal that is correlated with the amount of specifically bound viral particles. The color reaction was then stopped by adding Stop Solution. The absorbance at 450 nm was measured photometrically by a spectrophotometer.

Table 2 provides a detailed description of the inactivating mutations analyzed herein and in which AAV helper plasmid variant they appear. Members of the variant A family were generated using parent A as a backbone while members of the variant B family were generated using parent B as a backbone. The Adenovirus genomic regions in both parent A and B were derived from Ad5.

FIG. 1 provides an alignment of Variants A1 to A9 as compared to Parent A (the parent plasmid from which these variants were derived). The designation for each variant plasmid is provided at the left (A1, A2, and so on). The orientation and position of adenoviral genes, open reading frames (orfs), or other regions in Parent A are shown below the alignments in the labeled open arrows/boxes. Functional regions in the plasmid backbone or Patent A are shown in the labeled black/shaded arrows. The direction of the arrows indicates the directionality of each functional region, if it has one (e.g., direction of transcription). This figure illustrates the helper plasmids in a linear format to aid in showing their structural features and location of inactivation mutations even though they are circular plasmids when used for AAV production experiments in the Examples. Shaded regions in the linear representations of each variant are identical to Parent A. Vertical hashes in the linear representations represent point mutations or small insertions/deletions (less than 10 bp) in each variant while unshaded/open regions (i.e., white blocks) represent larger deletions. FIG. 2 is in the same format as FIG. 1 and provides an alignment of Variants B1 and B2 as compared to Parent B.

Together, the combined information in Table 2 (and the SEQ ID NOs referred to therein) and FIGS. 1 and 2 provides a detailed description of the inactivating mutations present in each variant assayed in the Examples.

TABLE 2

| ORF/Sequence | Mutation | |
|---|---|---|
| feature | [Description] | Helper constructs |
| Fiber | c.49delAT | VARIANT A5 |
| | [deletion of A at nucleotide position 49 and deletion of | |
| | T at nucleotide position 50; reference sequence - | |
| | SEQ ID NO: 3] | |
| Fiber | c.1_2AT > TA | VARIANT A4; VARIANT A6; |
| | [conversion of AT at nucleotide positions 1 and 2 to | VARIANT A7; VARIANT A8; |
| | TA, converting ATG start codon to TAG stop codon; | VARIANT A9 |
| | reference sequence - SEQ ID NO: 3] | |
| Fiber | c.22G > T | VARIANT A4; VARIANT A6; |
| | [G to T mutation at nucleotide position 22 to generate | VARIANT A7; VARIANT A8; |
| | premature stop codon; reference sequence - SEQ ID | VARIANT A9 |
| | NO: 3] | |
| Fiber | c.45T > A | VARIANT A4; VARIANT A6; |
| | [T to A mutation at nucleotide position 45; reference | VARIANT A7; VARIANT A8; |
| | sequence - SEQ ID NO: 3] | VARIANT A9 |
| Fiber | c.196_199ATGG > TAG | VARIANT A4; VARIANT A6; |
| | [Single base deletion (A at nucleotide position 196) | VARIANT A7; VARIANT A9 |
| | and point mutation (G to A mutation at nucleotide | |
| | position 198) to generate frameshift and premature | |
| | stop codon; reference sequence - SEQ ID NO: 3] | |
| Fiber | c.361-362AT > TA | VARIANT A4; VARIANT A6; |
| | [conversion of AT at nucleotide positions 361 and | VARIANT A7; VARIANT A9 |
| | 362 to destroy internal Met codon; reference | |
| | sequence - SEQ ID NO: 3] | |
| Fiber | c.385-386AT > TA | VARIANT A4; VARIANT A6; |
| | [conversion of AT at nucleotide positions 385 and | VARIANT A7; VARIANT A9 |
| | 386 to destroy internal Met codon; reference | |
| | sequence - SEQ ID NO: 3] | |
| Fiber | c.751-752AT > TA | VARIANT A4; VARIANT A6; |
| | [conversion of AT at nucleotide positions 751 and | VARIANT A7; VARIANT A9 |
| | 752 to destroy internal Met codon; reference | |
| | sequence - SEQ ID NO: 3] | |
| Fiber | c.1118delT | VARIANT A4; VARIANT A6; |
| | [deletion of T at nucleotide position 1118 to produce | VARIANT A7; VARIANT A9 |
| | frameshift mutation; reference sequence - SEQ ID | |
| | NO: 3] | |
| Fiber | c.1654-1655AT > TA | VARIANT A4; VARIANT A6; |
| | [conversion of AT at nucleotide positions 1654 and | VARIANT A7; VARIANT A9 |
| | 1655 to destroy internal Met codon; reference | |
| | sequence - SEQ ID NO: 3] | |
| pTP | pTP Deletion 1 | VARIANT A6; VARIANT A7; |
| | [deletion of SEQ ID NO: 6 to remove pTP ORF | VARIANT A8; VARIANT A9 |
| | sequence; the deleted sequence provided as SEQ ID | |
| | NO: 6 is in same orientation as pTP ORF, which is | |
| | opposite orientation to Fiber gene; reference | |
| | sequence - SEQ ID NO: 5] | |
| pTP | c.1T > A | VARIANT A6; VARIANT A7; |
| | [conversion of T to A at nucleotide position 1 to | VARIANT A8; VARIANT A9 |
| | destroy pTP start codon; reference sequence - SEQ | |
| | ID NO: 5] | |
| L1-52K | c.3G > A | VARIANT A6; VARIANT A7; |
| | [conversion of G to A at nucleotide position 3 to | VARIANT A8; VARIANT A9 |
| | destroy L1 52K start codon; reference sequence - | |
| | SEQ ID NO: 7] | |
| L1-52K | L1-52K Deletion 1 | VARIANT A6; VARIANT A7; |
| | [deletion of SEQ ID NO: 8 to remove L1 52K ORF | VARIANT A8; VARIANT A9 |
| | sequence] | |
| L1-52K; pIIIa; 23K | L1-52K Deletion 2 | VARIANT A3; VARIANT A6; |
| endoprotease | [deletion of SEQ ID NO: 9 to remove nonessential | VARIANT A8; VARIANT A9 |
| | sequences spanning multiple ORFs | |
| 100K | c.1847T > A | VARIANT A6; VARIANT A7; |
| | [conversion of T to A at nucleotide position 1847, | VARIANT A8 |
| | nonsense mutation to generate premature stop | |
| | codon; reference sequence - SEQ ID NO: 10] | |
| 100K | c.2026A > T | VARIANT A6; VARIANT A7; |
| | [conversion of T to A at nucleotide position 2026, | VARIANT A8 |
| | nonsense mutation to generate premature stop | |
| | codon; reference sequence - SEQ ID NO: 10] | |
| 100K | c.2239_2240AT > TA | VARIANT A6; VARIANT A7; |
| | [conversion of AT at nucleotide positions 2239 and | VARIANT A8 |
| | 2240 to TA, nonsense mutation to generate | |
| | premature stop codon; reference sequence - SEQ ID | |
| | NO: 10] | |
| 100K | c.2519_2520AT > TA | VARIANT A6; VARIANT A7; |
| | [conversion of AT at nucleotide positions 2519 and | VARIANT A8 |
| | 2520 to TA, nonsense mutation to generate | |
| | premature stop codon; reference sequence - SEQ ID | |
| | NO: 10] | |
| 100K | c.2685T > A | VARIANT A6; VARIANT A7; |
| | [conversion of T at nucleotide position 2685 to A, | VARIANT A8 |
| | nonsense mutation to generate premature stop | |
| | codon; reference sequence - SEQ ID NO: 10] | |
| Hexon-associated | c.1T > A | VARIANT A6; VARIANT A7; |
| precursor/pVIII | [conversion of T at nucleotide position 1 to A, | VARIANT A8 |
| | nonsense mutation to destroy start codon; reference | |
| | sequence - SEQ ID NO: 13] | |
| Hexon-associated | c.25_29TACAT > ATCTA | VARIANT A6; VARIANT A7; |
| precursor/pVIII | [conversion of TACAT sequence at nucleotide | VARIANT A8 |
| | positions 25-29 to ATCTA, converts internal Met | |
| | codon to stop codon; reference sequence - SEQ ID | |
| | NO: 13] | |
| Hexon-associated | c.49_50TA > AT | VARIANT A6; VARIANT A7; |
| precursor/pVIII | [conversion of TA at nucleotide positions 49 and 50 | VARIANT A8 |
| | AT, converts internal Met codon to stop codon; | |
| | reference sequence - SEQ ID NO: 13] | |
| Hexon-associated | c.100_101AT > TA | VARIANT A6; VARIANT A7; |
| precursor/pVIII | [conversion of AT at nucleotide positions 100 and | VARIANT A8 |
| | 101 to TA, converts internal Met codon to stop codon; | |
| | reference sequence - SEQ ID NO: 13] | |
| Fiber | Fiber Deletion 1 | VARIANT A1; VARIANT A3 |
| | [deletion of SEQ ID NO: 22; fiber sequence starting 5′ | |
| | to fiber gene (3′region of Hexon-associated precursor/ | |
| | pVIII), encompassing the entire fiber ORF; reference | |
| | sequence - SEQ ID NO: 3] | |
| Fiber | Fiber Deletion 2 | VARIANT A2 |
| | [deletion of SEQ ID NO: 23, fiber sequence starting 5′ | |
| | to fiber gene (3′region of Hexon-associated precursor/ | |
| | pVIII), encompassing the entire fiber ORF (alternate | |
| | sequence deletion); reference sequence - SEQ ID | |
| | NO: 3] | |
| E4orf1 | c.1_2TA > AT | VARIANT A4; VARIANT A6; |
| | [conversion of TA at nucleotide positions 1 and 2 to | VARIANT A7; VARIANT A9 |
| | AT, mutation to destroy E4orf1 start codon; reference | |
| | sequence - SEQ ID NO: 15] | |
| E4orf2 | c.1T > A | VARIANT A4; VARIANT A6; |
| | [conversion of T at nucleotide position 1 to A, | VARIANT A7; VARIANT A9 |
| | mutation to destroy E4orf2 start codon; reference | |
| | sequence - SEQ ID NO: 16] | |
| E4orf3 | c.1_2TA > AT | VARIANT A4; VARIANT A6; |
| | [conversion of TA at nucleotide positions 1 and 2 to | VARIANT A7; VARIANT A9 |
| | AT, mutation to destroy E4orf3 start codon; reference | |
| | sequence - SEQ ID NO: 17] | |
| E4orf4 | c.1_2TA > AT | VARIANT A4; VARIANT A6; |
| | [conversion of TA at nucleotide positions 1 and 2 to | VARIANT A7; VARIANT A9 |
| | AT, mutation to destroy E4orf4 start codon; reference | |
| | sequence - SEQ ID NO: 18] | |
| Ad5 ITR | Ad5 ITR Deletion 1 | VARIANT A4; VARIANT A6; |
| | [deletion of SEQ ID NO: 20, removing Ad5 ITR | VARIANT A7; VARIANT A9 |
| | sequence] | |
| Ad5 ITR | Ad5 ITR Deletion 2 | VARIANT A1; VARIANT A3 |
| | [deletion of SEQ ID NO: 21, removing Ad5 ITR | |
| | sequence (alternate sequence deletion)] | |
| 100K | c.3G > T | VARIANT B2; VARIANT B1 |
| | [conversion of G at nucleotide position 3 to T, | |
| | destroys start codon, silent in E2A DBP ORF; | |
| | reference sequence - SEQ ID NO: 10] | |
| 100K | c.312G > C | VARIANT B2; VARIANT B1 |
| | [conversion of G at nucleotide position 312 to C, | |
| | destroys downstream 100K Met codon, silent in E2A | |
| | DBP ORF; reference sequence - SEQ ID NO: 10] | |
| 100K | c.480G > T | VARIANT B2; VARIANT B1 |
| | [conversion of G at nucleotide position 480 to T, | |
| | destroys downstream 100K Met codon, silent in E2A | |
| | DBP ORF; reference sequence - SEQ ID NO: 10] | |
| Hexon assembly | c.1_3delATG | VARIANT B2; VARIANT B1 |
| (inside 100K) | [deletion of ATG at nucleotide positions 1-3, removes | |
| | start codon to destroy hexon assembly start codon; | |
| | reference sequence - SEQ ID NO: 11] | |
| Hexon assembly | c.933ins TAATAA | VARIANT B2; VARIANT B1 |
| (inside 100K) | [addition of TAATAA (SEQ ID NO: 12) at nucleotide | |
| | position 993, inserts tandem stop codon to prevent | |
| | hexon assembly expression; reference sequence - | |
| | SEQ ID NO: 11] | |
| Hexon Assembly | c.1937-1938delAT | VARIANT B2; VARIANT B1 |
| (begins at 22K | [deletion of AT at nucleotide positions 1937-1938, | |
| ORF) | destroys L4/22K potential ORF start codon, upstream | |
| | of TSS in E2A gene; reference sequence - SEQ ID | |
| | NO: 11] | |
| Hexon-associated | Hexon-associated precursor/pVIII Deletion 1 | VARIANT B2; VARIANT B1 |
| precursor/pVIII | [deletion of SEQ ID NO: 14, removes Hexon- | |
| | associated precursor/pVIII sequence; reference | |
| | sequence - SEQ ID NO: 13] | |
| E4orf1 | c.1_3delATG | VARIANT B1 |
| | [deletion of ATG at nucleotide positions 1-3, removes | |
| | start codon of E4orf1 (in frame to avoid E4 transcript | |
| | degradation); reference sequence - SEQ ID NO: 15] | |
| E4orf1; E4orf2; | E4 deletion 1 | VARIANT B2 |
| E4orf3; E4orf4 | [deletion of SEQ ID NO: 24, removes E4orf6 intron | |
| | sequence containing E4orf1, E4orf2, E4orf3, E4orf4 | |
| | start codons] | |
| E4orf2 | c.-18_-16delATG (upstream) | VARIANT B1 |
| | [deletion of ATG at nucleotide positions 18-16; | |
| | removes upstream Met (in frame to avoid E4 | |
| | transcript degradation); reference sequence - SEQ | |
| | ID NO: 16] | |
| E4orf2 | c.1_3delATG | VARIANT B1 |
| | [deletion of ATG at nucleotide positions 1-13; | |
| | removes E4orf2 start codon (in frame to avoid E4 | |
| | transcript degradation); reference sequence - SEQ | |
| | ID NO: 16] | |
| E4orf2 | c.16_18delATG | VARIANT B1 |
| | [deletion of ATG at nucleotide positions 16-18; | |
| | removes downstream Met (in frame to avoid E4 | |
| | transcript degradation); reference sequence - SEQ | |
| | ID NO: 16] | |
| E4orf3 | c.1_3delATG | VARIANT B1 |
| | [deletion of ATG at nucleotide positions 1-3; removes | |
| | E4orf3 start codon (in frame to avoid E4 transcript | |
| | degradation); reference sequence - SEQ ID NO: 17] | |
| E4orf3 | c.55_57delATG | VARIANT B1 |
| | [deletion of ATG at nucleotide positions 55-57, | |
| | removes E4orf3 downstream Met (in frame to avoid | |
| | E4 transcript degradation); reference sequence - | |
| | SEQ ID NO: 17] | |
| E4orf4 | c.1_3delATG | VARIANT B1 |
| | [deletion of ATG at nucleotide positions 1-3, removes | |
| | E4orf4 start codon (in frame to avoid E4 transcript | |
| | degradation); reference sequence - SEQ ID NO: 18] | |
| Fiber | Fiber deletion 3 | VARIANT B2 |
| | [deletion of SEQ ID NO: 25 in SEQ ID NO: 3, | |
| | removes residual non-coding fiber sequence] | |

Table 3 provides a listing of the inactivating mutations of Variants A1 to A9, B1, and B2 and the corresponding SEQ ID NO for the complete sequence of each Variant. As noted herein, the sequences for the Variants do not include plasmid backbone elements (e.g., the origin of replication, f1, antibiotic resistance gene, etc.).

TABLE 3

| Vari- | Inactivating Mutation from Table 2 | SEQ ID |
|---|---|---|
| ant | (ORF/feature if needed)) | NO |
| A1 | Fiber Deletion 1 | 37 |
| | Ad5 ITR Deletion 2 | |
| A2 | Fiber Deletion 2 | 38 |
| A3 | L1-52K Deletion 2 | 39 |
| | Fiber Deletion 1 | |
| | Ad5 ITR Deletion 2 | |
| A4 | c.1_2AT > TA (Fiber) | 40 |
| | c.22G > T (Fiber) | |
| | c.45T > A (Fiber) | |
| | c.196_199ATGG > TAG (Fiber) | |
| | c.361-362AT > TA (Fiber) | |
| | c.385-386AT > TA (Fiber) | |
| | c.751-752AT > TA (Fiber) | |
| | c.1118delT (Fiber) | |
| | c.1654-1655AT > TA (Fiber) | |
| | c.1_2TA > AT (E4orf1) | |
| | c.1T > A (E4orf2) | |
| | c.1_2TA > AT (E4orf3) | |
| | c.1_2TA > AT (E4orf4) | |
| | Ad5 ITR Deletion 1 | |
| A5 | c.49delAT (Fiber) | 41 |
| A6 | c.1_2AT > TA (Fiber) | 42 |
| | c.22G > T (Fiber) | |
| | c.45T > A (Fiber) | |
| | c.196_199ATGG > TAG (Fiber) | |
| | c.361-362AT > TA (Fiber) | |
| | c.385-386AT > TA (Fiber) | |
| | c.751-752AT > TA (Fiber) | |
| | c.1118delT (Fiber) | |
| | c.1654-1655AT > TA (Fiber) | |
| | pTP Deletion 1 | |
| | c.1T > A (pTP) | |
| | c.3G > A (L1-52K) | |
| | L1-52K Deletion 1 | |
| | L1-52K Deletion 2 | |
| | c.1847T > A (100K) | |
| | c.2026A > T (100K) | |
| | c.2239_2240AT > TA (100K) | |
| | c.2519_2520AT > TA (100K) | |
| | c.2685T > A (100K) | |
| | c.1T > A (Hexon-associated precursor/pVIII) | |
| | c.25_29TACAT > ATCTA (Hexon-associated | |
| | precursor/pVIII) | |
| | c.49_50TA > AT (Hexon-associated precursor/pVIII) | |
| | c.100_101AT > TA (Hexon-associated precursor/pVIII) | |
| | c.1_2TA > AT (E4orf1) | |
| | c.1T > A (E4orf2) | |
| | c.1_2TA > AT (E4orf3) | |
| | c.1_2TA > AT (E4orf4) | |
| | Ad5 ITR Deletion 1 | |
| A7 | c.1_2AT > TA (Fiber) | 43 |
| | c.22G > T (Fiber) | |
| | c.45T > A (Fiber) | |
| | c.196_199ATGG > TAG (Fiber) | |
| | c.361-362AT > TA (Fiber) | |
| | c.385-386AT > TA (Fiber) | |
| | c.751-752AT > TA (Fiber) | |
| | c.1118delT (Fiber) | |
| | c.1654-1655AT > TA (Fiber) | |
| | pTP Deletion 1 | |
| | c.1T > A (pTP) | |
| | c.3G > A (L1-52K) | |
| | L1-52K Deletion 1 | |
| | c.1847T > A (100K) | |
| | c.2026A > T (100K) | |
| | c.2239_2240AT > TA (100K) | |
| | c.2519_2520AT > TA (100K) | |
| | c.2685T > A (100K) | |
| | c.1T > A (Hexon-associated precursor/pVIII) | |
| | c.25_29TACAT > ATCTA (Hexon-associated | |
| | precursor/pVIII) | |
| | c.49_50TA > AT (Hexon-associated precursor/pVIII) | |
| | c.100_101AT > TA (Hexon-associated precursor/pVIII) | |
| | c.1_2TA > AT (E4orf1) | |
| | c.1T > A (E4orf2) | |
| | c.1_2TA > AT (E4orf3) | |
| | c.1_2TA > AT (E4orf4) | |
| | Ad5 ITR Deletion 1 | |
| A8 | c.1_2AT > TA (Fiber) | 44 |
| | c.22G > T (Fiber) | |
| | c.45T > A (Fiber) | |
| | pTP Deletion 1 | |
| | c.1T > A (pTP) | |
| | c.3G > A (L1-52K) | |
| | L1-52K Deletion 1 | |
| | L1-52K Deletion 2 | |
| | c.1847T > A (100K) | |
| | c.2026A > T (100K) | |
| | c.2239_2240AT > TA (100K) | |
| | c.2519_2520AT > TA (100K) | |
| | c.2685T > A (100K) | |
| | c.1T > A (Hexon-associated precursor/pVIII) | |
| | c.25_29TACAT > ATCTA (Hexon-associated | |
| | precursor/pVIII) | |
| | c.49_50TA > AT (Hexon-associated precursor/pVIII) | |
| | c.100_101AT > TA (Hexon-associated precursor/pVIII) | |
| A9 | c.1_2AT > TA (Fiber) | 45 |
| | c.22G > T (Fiber) | |
| | c.45T > A (Fiber) | |
| | c.196_199ATGG > TAG (Fiber) | |
| | c.361-362AT > TA (Fiber) | |
| | c.385-386AT > TA (Fiber) | |
| | c.751-752AT > TA (Fiber) | |
| | c.1118delT (Fiber) | |
| | c.1654-1655AT > TA (Fiber) | |
| | pTP Deletion 1 | |
| | c.1T > A (pTP) | |
| | c.3G > A (L1-52K) | |
| | L1-52K Deletion 1 | |
| | L1-52K Deletion 2 | |
| | c.1_2TA > AT (E4orf1) | |
| | c.1T > A (E4orf2) | |
| | c.1_2TA > AT (E4orf3) | |
| | c.1_2TA > AT (E4orf4) | |
| | Ad5 ITR Deletion 1 | |
| B1 | c.3G > T (100K) | 46 |
| | c.312G > C (100K) | |
| | c.480G > T (100K) | |
| | c.1_3delATG (Hexon assembly; inside 100K) | |
| | c.933insTAATAA (Hexon assembly; inside 100K) | |
| | c.1937-1938delAT (Hexon assembly; at 22K) | |
| | Hexon-associated precursor/pVIII Deletion 1 | |
| | c.1_3delATG (E4orf1) | |
| | c.-18_-16delATG (upstream) (E4orf2) | |
| | c.1_3delATG (E4orf2) | |
| | c.16_18delATG (E4orf2) | |
| | c.1_3delATG (E4orf3) | |
| | c.55_57delATG (E4orf3) | |
| | c.1_3delATG (E4orf4) | |
| B2 | c.3G > T (100K) | 47 |
| | c.312G > C (100K) | |
| | c.480G > T (100K) | |
| | c.1_3delATG (Hexon assembly; inside 100K) | |
| | c.933insTAATAA (Hexon assembly; inside 100K) | |
| | c.1937-1938delAT (Hexon assembly; at 22K) | |
| | Hexon-associated precursor/pVIII Deletion 1 | |
| | E4 deletion 1 | |
| | Fiber deletion 3 | |

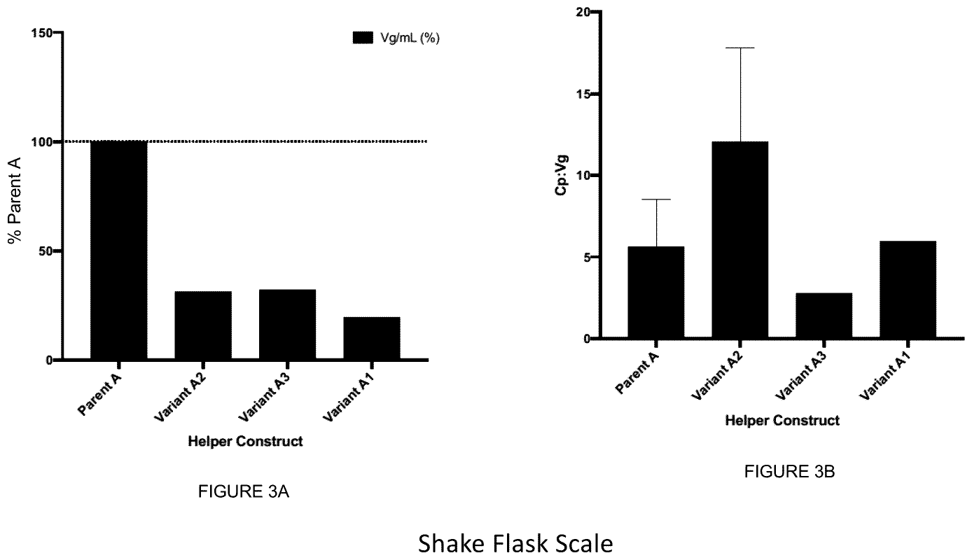

The results of the methods described above are illustrated in FIGS. 3-6.

### Example 2: Fiber Gene Deletion Reduces AAV Production

Shake flask AAV production experiments (30 mL volume) were performed to compare AAV production in viral genomes per mL of harvested lysate (Vg/mL) and capsid protein to viral genome ratios (Cp:Vg) between Parent A and Variants A1, A2, and A3, each of which had the fiber gene deleted (fiber deletion 1 for Variants A1 and A3; fiber gene deletion 2 for Variant A2; see Table 2). FIG. 1 shows a schematic representation. As shown in FIG. 3A, Vg/mL in each of these three variants fell dramatically as compared to Parent A. The Cp:Vg ratios for Variant A2 increased while those for Variants A3 and A1 were similar or slightly lower than Parent A (FIG. 3B).

These results indicate that deletion of a region including the entire fiber gene from Parent A negatively impacts AAV vector production. Based on our current understanding of AAV vector production, the fiber protein itself is not a necessary component for AAV production. As such, these experiments indicate that the region deleted in Variants A1-A3 provides a benefit to AAV vector production efficiency that is not tied to the production of the fiber protein itself. While not being bound by theory, this region may include a genetic element (e.g., a regulatory element) that impacts the expression or activity of other adenoviral helper genes and/or AAV genes that play a role in AAV vector production.

### Example 3: Variants with Alternative Fiber Gene Inactivating Mutations Improve AAV Production

Shake flask AAV production experiments (30 mL volume) were performed to compare AAV production in viral genomes per mL of harvested lysate (Vg/mL) and capsid protein to viral genome ratios (Cp:Vg) between Parent A and Variants A4, A5, A6, A7, A8, and A9. Each of these variants include fiber gene inactivating mutations that are not deletions of the entire fiber gene (as are the deletions in variants A1, A2, and A3). As such, if the fiber gene region provides a benefit to AAV production that is independent of the expression of the fiber gene itself, these variants would be expected to result in higher AAV titers than variants with complete deletion of the fiber gene region (e.g., variants A1-A3) when used as helper plasmids in AAV production. Each variant tested in this example, apart from variant A5, includes additional inactivating mutations in different genes/regions of the helper plasmid. Variant A4 includes additional inactivating mutations in the E4 region orfs and the ITR; Variant A6 includes additional inactivating mutations in pTP, the L1-52K region, 100K, PVIII, the E4 region orfs, and the ITR; Variant A7 includes additional inactivating mutations in pTP, L1-52K, 100K, PVIII, the E4 region orfs, and the ITR; Variant A8 includes additional inactivating mutations in pTP, the L1-52K region, 100K, and PVIII; and Variant A9 includes additional inactivating mutations in: pTP, the L1-52K region, PVIII, the E4 region orfs, and the ITR. The specific inactivating mutations present in each of these variants is listed in Table 2, described in detail in hereinabove, and shown schematically in FIG. 1.

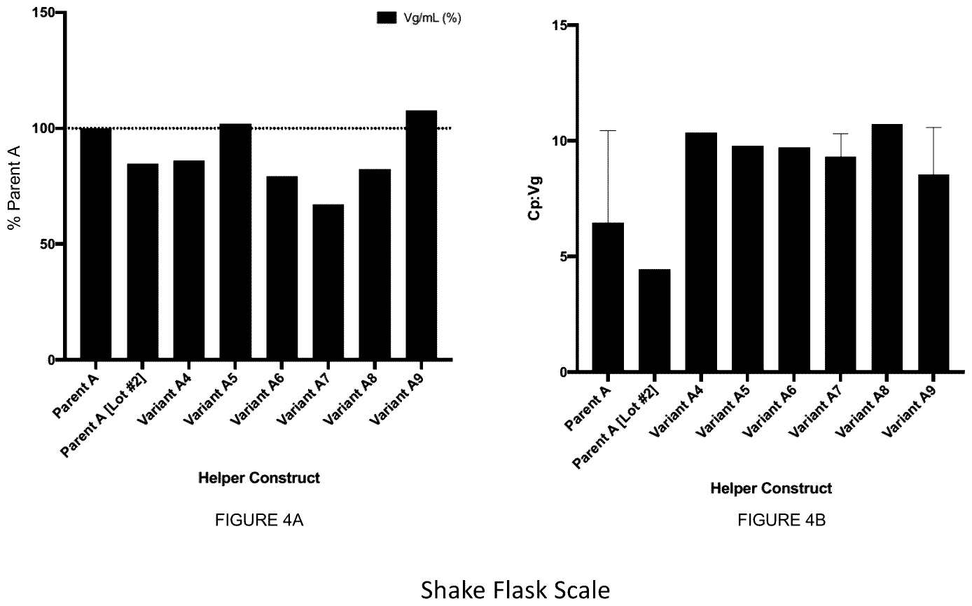

As shown in FIG. 4A, AAV titers achieved using each of variants A4 to A9 as helper plasmids in the shake flask production assay were higher than those achieved for variants A1-A3 (see FIG. 3A). Specifically, AAV titers achieved using variants A4-A9 were at least 60% of AAV titers achieved with Parent A (the results from two different lots of Parent A are shown in FIG. 3A). Variants A4, A5, and A9 achieved AAV titers that were equivalent to Parent A. As shown in FIG. 4B, the Cp:Vg ratio for each variant tested was approximately 10, which is higher than the parent. This indicates that use of these variants in AAV production may result in a slightly higher level of empty capsids production in the host cells than the parent. However, the improved safety profile of these variants, i.e., the inactivation of the fiber gene and, in some variants, several additional genes/regions, is enough to overcome this minor inefficiency with respect to their use in generating clinical-grade AAV drug substance.

### Example 4: Inactivating Mutations in Different Parent Helper Plasmid

Shake flask AAV production experiments (30 mL volume) were performed to compare AAV production in viral genomes per mL of harvested lysate (Vg/mL) and capsid protein to viral genome ratios (Cp:Vg) between Parent B and Variants B1 and B2. Parent B does not include an adenoviral ITR or a functional fiber gene (only a residual fiber fragment is present). Variant B1 includes inactivating mutations in 100 k, PVIII, Hexon Assembly, and the E4 region orfs; Variant B2 includes inactivating mutations in 100 k, PVIII, Hexon Assembly, the E4 region orfs (deletion), and has a deletion removing the residual fiber gene fragment. The specific inactivating mutations present in each of these variants is listed in Table 2, described in detail herein, and shown schematically in FIG. 2.

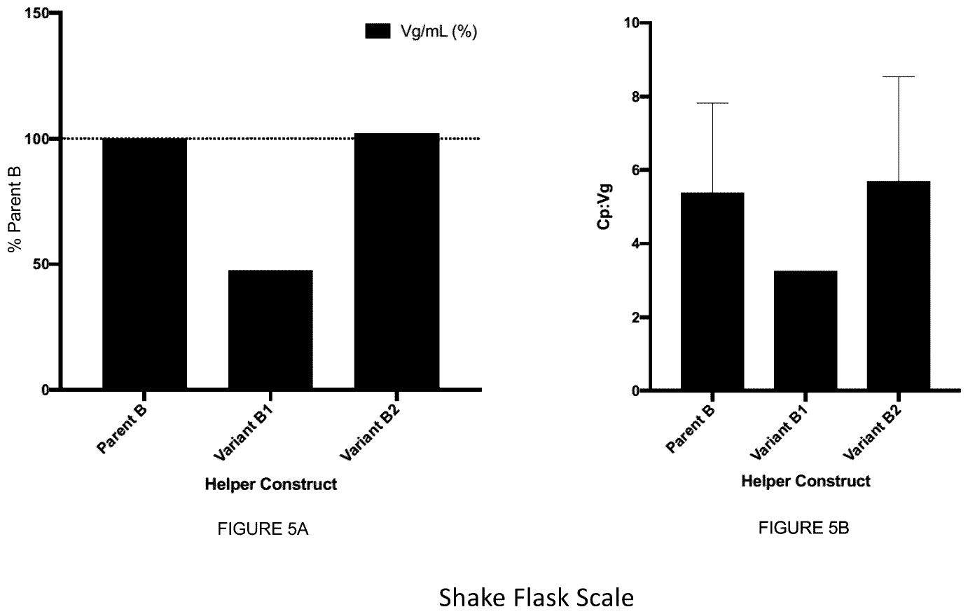

As shown in FIG. 5A, the AAV titer achieved using Variant B1 as a helper plasmid in the shake flask production assay was approximately 50% of the titer achieved with Parent B. In contrast, the titer achieved using Variant B2 was equivalent to Parent B. As shown in FIG. 5B, the Cp:Vg ratio for these variants was lower (Variant B1) or equivalent (Variant B2) to the parent. As compared to Parent B, Variant B2 has an improved safety profile while maintaining equivalent AAV production performance. These characteristics make it a good candidate for use as an AAV helper plasmid in generating clinical-grade AAV drug substance.

### Example 5: Bioreactor Scale AAV Production Assays

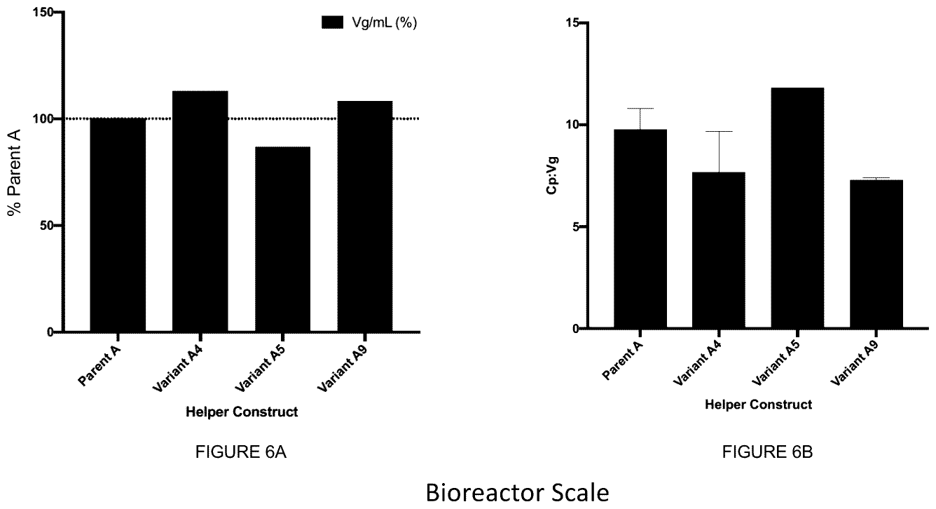

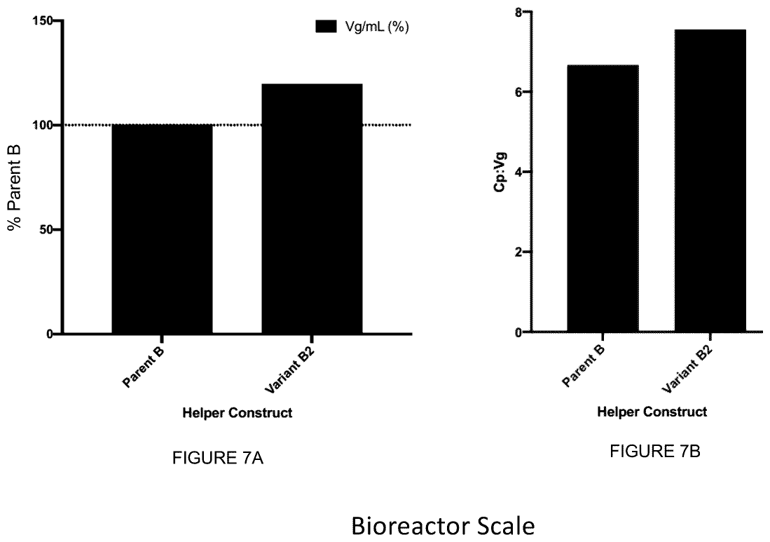

Bioreactor scale AAV production experiments (2.0 L) were performed to compare AAV production in viral genomes per mL of harvested lysate (Vg/mL) and capsid protein to viral genome ratios (Cp:Vg) between specific variant helper plasmids and their respective parent helper plasmids. FIGS. 6A and 6B show the results for Variants A4, A5, and A9 as compared to Parent A while FIGS. 7A and 7B show the results for Variant B2 as compared to Parent B. As shown in these figures, each of the variants performed equivalently to their respective parent helper plasmids with respect to both viral titer (FIGS. 6A and 7A) and Cp:Vg ratio (FIGS. 6B and 7B). These results demonstrate that AAV helper variant plasmids A4, A5, A9, and B2 maintain their AAV production characteristics at increased scale. Given their improved safety profiles, each of these AAV helper variant plasmids are good candidates for use in generating clinical-grade AAV drug substance.

### Example 6: Fiber Gene Transcript Expression in Host Cells

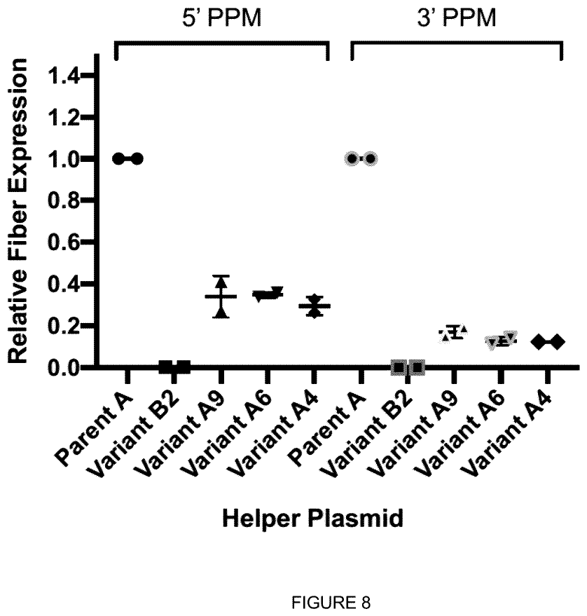

FIG. 8 shows the results of fiber gene transcript qRT-PCR assays performed in HEK293 cells (as described above in Example 1) for the AAV helper plasmids indicated. All variant plasmids are compared to Parent A, which includes a functional fiber gene. Variant B2, which does not include a functional fiber gene, was included as a negative control. Primer sets directed to a 5′ region of the fiber gene transcript (5′ PPM) and a 3′ region of the fiber gene transcript (3′ PPM) were used as indicated at the top of the graph in FIG. 8.

As shown in FIG. 8, the level of fiber gene transcript present in the HEK293 host cells was significantly reduced in Variants A4, A6, and A9, indicating that the inactivating mutations in the fiber gene in these variants resulted in lower transcription of the fiber gene, lower stability of the fiber gene transcript, or both. This reduced level of fiber gene transcript in the host cells provides for an improved safety profile for the AAV product as compared to Parent A. For example, using any one of these variant helper plasmids for AAV production will reduce the expression of a protein or protein fragment from these fiber gene transcripts. In addition, should off-target fiber gene DNA get packaged into an AAV capsid during the production process, any possible transcript expressed from this off-target DNA when administered to a subject (e.g., a human subject) would be reduced or eliminated.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature. The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 10%, up to 5%, or up to 1% of a given value.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. In any of the ranges described herein, the endpoints of the range are included in the range. However, the description also contemplates the same ranges in which the lower and/or the higher endpoint is excluded.

All method steps described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

TABLE 4
Sequences

| SEQ ID | | |
|---|---|---|
| NO | Description | Sequence |
| 1 | Parent A; | GCGAAAGGGGGATGTGCTGCAAGGCGATTAAGTTGGGTAACGCCAGGGTTTT |
| | without | CCCAGTCACGACGTTGTAAAACGACGGCCAGTGCCAAGCTTAAGGTGCACGG |
| | plasmid | CCCACGTGGCCACTAGTACTTCTCGACAGAAGCACCATGTCCTTGGGTCCGG |
| | backbone | CCTGCTGAATGCGCAGGCGGTCGGCCATGCCCCAGGCTTCGTTTTGACATCG |
| | elements | GCGCAGGTCTTTGTAGTAGTCTTGCATGAGCCTTTCTACCGGCACTTCTTCTT |
| | (e.g., | CTCCTTCCTCTTGTCCTGCATCTCTTGCATCTATCGCTGCGGCGGCGGCGGA |
| | antibiotic | GTTTGGCCGTAGGTGGCGCCCTCTTCCTCCCATGCGTGTGACCCCGAAGCCC |
| | resistance | CTCATCGGCTGAAGCAGGGCTAGGTCGGCGACAACGCGCTCGGCTAATATG |
| | gene, ori) | GCCTGCTGCACCTGCGTGAGGGTAGACTGGAAGTCATCCATGTCCACAAAGC |
| | | GGTGGTATGCGCCCGTGTTGATGGTGTAAGTGCAGTTGGCCATAACGGACCA |
| | | GTTAACGGTCTGGTGACCCGGCTGCGAGAGCTCGGTGTACCTGAGACGCGA |
| | | GTAAGCCCTCGAGTCAAATACGTAGTCGTTGCAAGTCCGCACCAGGTACTGG |
| | | TATCCCACCAAAAAGTGCGGGGGGGGCTGGCGGTAGAGGGGCCAGCGTAGG |
| | | GTGGCCGGGGCTCCGGGGGCGAGATCTTCCAACATAAGGCGATGATATCCG |
| | | TAGATGTACCTGGACATCCAGGTGATGCCGGGGGGGGTGGTGGAGGCGCGC |
| | | GGAAAGTCGCGGACGCGGTTCCAGATGTTGCGCAGCGGCAAAAAGTGCTCC |
| | | ATGGTCGGGACGCTCTGGCCGGTCAGGCGCGCGCAATCGTTGACGCTCTAG |
| | | CGTGCAAAAGGAGAGCCTGTAAGCGGGCACTCTTCCGTGGTCTGGTGGATAA |
| | | ATTCGCAAGGGTATCATGGCGGACGACCGGGGTTCGAGCCCCGTATCCGGC |
| | | CGTCCGCCGTGATCCATGCGGTTACCGCCCGCGTGTCGAACCCAGGTGTGC |
| | | GACGTCAGACAACGGGGGAGTGCTCCTTTTGGCTTCCTTCCAGGCGCGGCG |
| | | GCTGCTGCGCTAGCTTTTTTGGCCACTGGCCGCGCGCAGCGTAAGCGGTTAG |
| | | GCTGGAAAGCGAAAGCATTAAGTGGCTCGCTCCCTGTAGCCGGAGGGTTATT |
| | | TTCCAAGGGTTGAGTCGCGGGACCCCCGGTTCGAGTCTCGGACCGGCCGGA |
| | | CTGCGGCGAACGGGGGTTTGCCTCCCCGTCATGCAAGACCCCGCTTGCAAAT |
| | | TCCTCCGGAAACAGGGACGAGCCCCTTTTTTGCTTTTCCCAGATGCATCCGGT |
| | | GCTGCGGCAGATGCGCCCCCCTCCTCAGCAGCGGCAAGAGCAAGAGCAGCG |
| | | GCAGACATGCAGGGCACCCTCCCCTCCTCCTACCGCGTCAGGAGGGGCGAC |
| | | ATCCGCGGTTGACGCGGCAGCAGATGGTGATTACGAACCCCCGCGGCGCCG |
| | | GGCCCGGCACTACCTGGACTTGGAGGAGGGCGAGGGCCTGGCGCGGCTAG |
| | | GAGCGCCCTCTCCTGAGCGGCACCCAAGGGTGCAGCTGAAGCGTGATACGC |
| | | GTGAGGCGTACGTGCCGCGGCAGAACCTGTTTCGCGACCGCGAGGGAGAGG |
| | | AGCCCGAGGAGATGCGGGATCGAAAGTTCCACGCAGGGCGCGAGCTGCGGC |
| | | ATGGCCTGAATCGCGAGCGGTTGCTGCGCGAGGAGGACTTTGAGCCCGACG |
| | | CGCGAACCGGGATTAGTCCCGCGCGCGCACACGTGGCGGCCGCCGACCTG |
| | | GTAACCGCATACGAGCAGACGGTGAACCAGGAGATTAACTTTCAAAAAAGCTT |
| | | TAACAACCACGTGCGTACGCTTGTGGCGCGCGAGGAGGTGGCTATAGGACT |
| | | GATGCATCTGTGGGACTTTGTAAGCGCGCTGGAGCAAAACCCAAATAGCAAG |
| | | CCGCTCATGGCGCAGCTGTTCCTTATAGTGCAGCACAGCAGGGACAACGAGG |
| | | CATTCAGGGATGCGCTGCTAAACATAGTAGAGCCCGAGGGCCGCTGGCTGCT |
| | | CGATTTGATAAACATCCTGCAGAGCATAGTGGTGCAGGAGCGCAGCTTGAGC |
| | | CTGGCTGACAAGGTGGCCGCCATCAACTATTCCATGCTTAGCCTGGGCAAGT |
| | | TTTACGCCCGCAAGATATACCATACCCCTTACGTTCCCATAGACAAGGAGGTA |
| | | AAGATCGAGGGGTTCTACATGCGCATGGCGCTGAAGGTGCTTACCTTGAGCG |
| | | ACGACCTGGGCGTTTATCGCAACGAGCGCATCCACAAGGCCGTGAGCGTGA |
| | | GCCGGCGGCGCGAGCTCAGCGACCGCGAGCTGATGCACAGCCTGCAAAGG |
| | | GCCCTGGCTGGCACGGGCAGCGGCGATAGAGAGGCCGAGTCCTACTTTGAC |
| | | GCGGGCGCTGACCTGCGCTGGGCCCCAAGCCGACGCGCCCTGGAGGCAGC |
| | | TGGGGCCGGACCTGGGCTGGCGGTGGCACCCGCGCGCGCTGGCAACGTCG |
| | | GCGGCGTGGAGGAATATGACGAGGACGATGAGTACGAGCCAGAGGACGGCG |
| | | AGTACTAAGCGGTGATGTTTCTGATCAGATGATGCAAGACGCAACGGACCCG |
| | | GCGGTGCGGGCGGCGCTGCAGAGCCAGCCGTCCGGCCTTAACTCCACGGAC |
| | | GACTGGCGCCAGGTCATGGACCGCATCATGTCGCTGACTGCGCGCAATCCTG |
| | | ACGCGTTCCGGCAGCAGCCGCAGGCCAACCGGCTCTCCGCAATTCTGGAAG |
| | | CGGTGGTCCCGGCGCGCGCAAACCCCACGCACGAGAAGGTGCTGGCGATCG |
| | | TAAACGCGCTGGCCGAAAACAGGGCCATCCGGCCCGACGAGGCCGGCCTGG |
| | | TCTACGACGCGCTGCTTCAGCGCGTGGCTCGTTACAACAGCGGCAACGTGCA |
| | | GACCAACCTGGACCGGCTGGTGGGGGATGTGCGCGAGGCCGTGGCGCAGC |
| | | GTGAGCGCGCGCAGCAGCAGGGCAACCTGGGCTCCATGGTTGCACTAAACG |
| | | CCTTCCTGAGTACACAGCCCGCCAACGTGCCGCGGGGACAGGAGGACTACA |
| | | CCAACTTTGTGAGCGCACTGCGGCTAATGGTGACTGAGACACCGCAAAGTGA |
| | | GGTGTACCAGTCTGGGCCAGACTATTTTTTCCAGACCAGTAGACAAGGCCTG |
| | | CAGACCGTAAACCTGAGCCAGGCTTTCAAAAACTTGCAGGGGCTGTGGGGGG |
| | | TGCGGGCTCCCACAGGCGACCGCGCGACCGTGTCTAGCTTGCTGACGCCCA |
| | | ACTCGCGCCTGTTGCTGCTGCTAATAGCGCCCTTCACGGACAGTGGCAGCGT |
| | | GTCCCGGGACACATACCTAGGTCACTTGCTGACACTGTACCGCGAGGCCATA |
| | | GGTCAGGCGCATGTGGACGAGCATACTTTCCAGGAGATTACAAGTGTCAGCC |
| | | GCGCGCTGGGGCAGGAGGACACGGGCAGCCTGGAGGCAACCCTAAACTACC |
| | | TGCTGACCAACCGGCGGCAGAAGATCCCCTCGTTGCACAGTTTGCACCCTTT |
| | | GGCGCATCCCATTCTCCAGTAACTTTATGTCCATGGGCGCACTCACAGACCTG |
| | | GGCCAAAACCTTCTCTACGCCAACTCCGCCCACGCGCTAGACATGACTTTTGA |
| | | GGTGGATCCCATGGACGAGCCCACCCTTCTTTATGTTTTGTTTGAAGTCTTTG |
| | | ACGTGGTCCGTGTGCACCAGCCGCACCGCGGCGTCATCGAAACCGTGTACC |
| | | TGCGCACGCCCTTCTCGGCCGGCAACGCCACAACATAAAGAAGCAAGCAACA |
| | | TCAACAACAGCTGCCGCCATGGGCTCCAGTGAGCAGGAACTGAAAGCCATTG |
| | | TCAAAGATCTTGGTTGTGGGCCATATTTTTTGGGCACCTATGACAAGCGCTTT |
| | | CCAGGCTTTGTTTCTCCACACAAGCTCGCCTGCGCCATAGTCAATACGGCCG |
| | | GTCGCGAGACTGGGGGCGTACACTGGATGGCCTTTGCCTGGAACCCGCACT |
| | | CAAAAACATGCTACCTCTTTGAGCCCTTTGGCTTTTCTGACCAGCGACTCAAG |
| | | CAGGTTTACCAGTTTGAGTACGAGTCACTCCTGCGCCGTAGCGCCATTGCTTC |
| | | TTCCCCCGACCGCTGTATAACGCTGGAAAAGTCCACCCAAAGCGTACAGGGG |
| | | CCCAACTCGGCCGCCTGTGGACTATTCTGCTGCATGTTTCTCCACGCCTTTGC |
| | | CAACTGGCCCCAAACTCCCATGGATCACAACCCCACCATGAACCTTATTACCG |
| | | GGGTACCCAACTCCATGCTCAACAGTCCCCAGGTACAGCCCACCCTGCGTCG |
| | | CAACCAGGAACAGCTCTACAGCTTCCTGGAGCGCCACTCGCCCTACTTCCGC |
| | | AGCCACAGTGCGCAGATTAGGAGCGCCACTTCTTTTTGTCACTTGAAAAACAT |
| | | GTAAAAATAATGTACTAGAGACACTTTCAATAAAGGCAAATGCTTTTATTTGTA |
| | | CACTCTCGGGTGATTATTTACCCCCACCCTTGCCGTCTGCGCCGTTTAAAAAT |
| | | CAAAGGGGTTCTGCCGCGCATCGCTATGCGCCACTGGCAGGGACACGTTGC |
| | | GATACTGGTGTTTAGTGCTCCACTTAAACTCAGGCACAACCATCCGCGGCAG |
| | | CTCGGTGAAGTTTTCACTCCACAGGCTGCGCACCATCACCAACGCGTTTAGC |
| | | AGGTCGGGCGCCGATATCTTGAAGTCGCAGTTGGGGCCTCCGCCCTGCGCG |
| | | CGCGAGTTGCGATACACAGGGTTGCAGCACTGGAACACTATCAGCGCCGGGT |
| | | GGTGCACGCTGGCCAGCACGCTCTTGTCGGAGATCAGATCCGCGTCCAGGT |
| | | CCTCCGCGTTGCTCAGGGCGAACGGAGTCAACTTTGGTAGCTGCCTTCCCAA |
| | | AAAGGGCGCGTGCCCAGGCTTTGAGTTGCACTCGCACCGTAGTGGCATCAAA |
| | | AGGTGACCGTGCCCGGTCTGGGCGTTAGGATACAGCGCCTGCATAAAAGCCT |
| | | TGATCTGCTTAAAAGCCACCTGAGCCTTTGCGCCTTCAGAGAAGAACATGCCG |
| | | CAAGACTTGCCGGAAAACTGATTGGCCGGACAGGCCGCGTCGTGCACGCAG |
| | | CACCTTGCGTCGGTGTTGGAGATCTGCACCACATTTCGGCCCCACCGGTTCT |
| | | TCACGATCTTGGCCTTGCTAGACTGCTCCTTCAGCGCGCGCTGCCCGTTTTC |
| | | GCTCGTCACATCCATTTCAATCACGTGCTCCTTATTTATCATAATGCTTCCGTG |
| | | TAGACACTTAAGCTCGCCTTCGATCTCAGCGCAGCGGTGCAGCCACAACGCG |
| | | CAGCCCGTGGGCTCGTGATGCTTGTAGGTCACCTCTGCAAACGACTGCAGGT |
| | | ACGCCTGCAGGAATCGCCCCATCATCGTCACAAAGGTCTTGTTGCTGGTGAA |
| | | GGTCAGCTGCAACCCGCGGTGCTCCTCGTTCAGCCAGGTCTTGCATACGGCC |
| | | GCCAGAGCTTCCACTTGGTCAGGCAGTAGTTTGAAGTTCGCCTTTAGATCGTT |
| | | ATCCACGTGGTACTTGTCCATCAGCGCGCGCGCAGCCTCCATGCCCTTCTCC |
| | | CACGCAGACACGATCGGCACACTCAGCGGGTTCATCACCGTAATTTCACTTTC |
| | | CGCTTCGCTGGGCTCTTCCTCTTCCTCTTGCGTCCGCATACCACGCGCCACT |
| | | GGGTCGTCTTCATTCAGCCGCCGCACTGTGCGCTTACCTCCTTTGCCATGCTT |
| | | GATTAGCACCGGTGGGTTGCTGAAACCCACCATTTGTAGCGCCACATCTTCTC |
| | | TTTCTTCCTCGCTGTCCACGATTACCTCTGGTGATGGGGGGCGCTCGGGCTT |
| | | GGGAGAAGGGCGCTTCTTTTTCTTCTTGGGCGCAATGGCCAAATCCGCCGCC |
| | | GAGGTCGATGGCCGCGGGCTGGGTGTGCGCGGCACCAGCGCGTCTTGTGAT |
| | | GAGTCTTCCTCGTCCTCGGACTCGATACGCCGCCTCATCCGCTTTTTTGGGG |
| | | GCGCCCGGGGAGGCGGCGGCGACGGGGACGGGGACGACACGTCCTCCATG |
| | | GTTGGGGGACGTCGCGCCGCACCGCGTCCGCGCTCGGGGGTGGTTTCGCG |
| | | CTGCTCCTCTTCCCGACTGGCCATTTCCTTCTCCTATAGGCAGAAAAAGATCA |
| | | TGGAGTCAGTCGAGAAGAAGGACAGCCTAACCGCCCCCTCTGAGTTCGCCAC |
| | | CACCGCCTCCACCGATGCCGCCAACGCGCCTACCACCTTCCCCGTCGAGGC |
| | | ACCCCCGCTTGAGGAGGAGGAAGTGATTATCGAGCAGGACCCAGGTTTTGTA |
| | | AGCGAAGACGACGAGGACCGCTCAGTACCAACAGAGGATAAAAAGCAAGACC |
| | | AGGACAACGCAGAGGCAAACGAGGAACAAGTCGGGGGGGGGACGAAAGG |
| | | CATGGCGACTACCTAGATGTGGGAGACGACGTGCTGTTGAAGCATCTGCAGC |
| | | GCCAGTGCGCCATTATCTGCGACGCGTTGCAAGAGCGCAGCGATGTGCCCCT |
| | | CGCCATAGCGGATGTCAGCCTTGCCTACGAACGCCACCTATTCTCACCGCGC |
| | | GTACCCCCCAAACGCCAAGAAAACGGCACATGCGAGCCCAACCCGCGCCTC |
| | | AACTTCTACCCCGTATTTGCCGTGCCAGAGGTGCTTGCCACCTATCACATCTT |
| | | TTTCCAAAACTGCAAGATACCCCTATCCTGCCGTGCCAACCGCAGCCGAGCG |
| | | GACAAGCAGCTGGCCTTGCGGCAGGGCGCTGTCATACCTGATATCGCCTCGC |
| | | TCAACGAAGTGCCAAAAATCTTTGAGGGTCTTGGACGCGACGAGAAGCGCGC |
| | | GGCAAACGCTCTGCAACAGGAAAACAGCGAAAATGAAAGTCACTCTGGAGTG |
| | | TTGGTGGAACTCGAGGGTGACAACGCGCGCCTAGCCGTACTAAAACGCAGCA |
| | | TCGAGGTCACCCACTTTGCCTACCCGGCACTTAACCTACCCCCCAAGGTCAT |
| | | GAGCACAGTCATGAGTGAGCTGATCGTGCGCCGTGCGCAGCCCCTGGAGAG |
| | | GGATGCAAATTTGCAAGAACAAACAGAGGAGGGCCTACCCGCAGTTGGCGAC |
| | | GAGCAGCTAGCGCGCTGGCTTCAAACGCGCGAGCCTGCCGACTTGGAGGAG |
| | | CGACGCAAACTAATGATGGCCGCAGTGCTCGTTACCGTGGAGCTTGAGTGCA |
| | | TGCAGCGGTTCTTTGCTGACCCGGAGATGCAGCGCAAGCTAGAGGAAACATT |
| | | GCACTACACCTTTCGACAGGGCTACGTACGCCAGGCCTGCAAGATCTCCAAC |
| | | GTGGAGCTCTGCAACCTGGTCTCCTACCTTGGAATTTTGCACGAAAACCGCCT |
| | | TGGGCAAAACGTGCTTCATTCCACGCTCAAGGGCGAGGCGCGCCGCGACTA |
| | | CGTCCGCGACTGCGTTTACTTATTTCTATGCTACACCTGGCAGACGGCCATGG |
| | | GCGTTTGGCAGCAGTGCTTGGAGGAGTGCAACCTCAAGGAGCTGCAGAAACT |
| | | GCTAAAGCAAAACTTGAAGGACCTATGGACGGCCTTCAACGAGCGCTCCGTG |
| | | GCCGCGCACCTGGCGGACATCATTTTCCCCGAACGCCTGCTTAAAACCCTGC |
| | | AACAGGGTCTGCCAGACTTCACCAGTCAAAGCATGTTGCAGAACTTTAGGAAC |
| | | TTTATCCTAGAGCGCTCAGGAATCTTGCCCGCCACCTGCTGTGCACTTCCTAG |
| | | CGACTTTGTGCCCATTAAGTACCGCGAATGCCCTCCGCCGCTTTGGGGCCAC |
| | | TGCTACCTTCTGCAGCTAGCCAACTACCTTGCCTACCACTCTGACATAATGGA |
| | | AGACGTGAGCGGTGACGGTCTACTGGAGTGTCACTGTCGCTGCAACCTATGC |
| | | ACCCCGCACCGCTCCCTGGTTTGCAATTCGCAGCTGCTTAACGAAAGTCAAAT |
| | | TATCGGTACCTTTGAGCTGCAGGGTCCCTCGCCTGACGAAAAGTCCGCGGCT |
| | | CCGGGGTTGAAACTCACTCCGGGGCTGTGGACGTCGGCTTACCTTCGCAAAT |
| | | TTGTACCTGAGGACTACCACGCCCACGAGATTAGGTTCTACGAAGACCAATCC |
| | | CGCCCGCCTAATGCGGAGCTTACCGCCTGCGTCATTACCCAGGGCCACATTC |
| | | TTGGCCAATTGCAAGCCATCAACAAAGCCCGCCAAGAGTTTCTGCTACGAAAG |
| | | GGACGGGGGGTTTACTTGGACCCCCAGTCCGGCGAGGAGCTCAACCCAATC |
| | | CCCCCGCCGCCGCAGCCCTATCAGCAGCAGCCGCGGGCCCTTGCTTCCCAG |
| | | GATGGCACCCAAAAAGAAGCTGCAGCTGCCGCCGCCACCCACGGACGAGGA |
| | | GGAATACTGGGACAGTCAGGCAGAGGAGGTTTTGGACGAGGAGGAGGAGGA |
| | | CATGATGGAAGACTGGGAGAGCCTAGACGAGGAAGCTTCCGAGGTCGAAGA |
| | | GGTGTCAGACGAAACACCGTCACCCTCGGTCGCATTCCCCTCGCCGGCGCC |
| | | CCAGAAATCGGCAACCGGTTCCAGCATGGCTACAACCTCCGCTCCTCAGGCG |
| | | CCGCCGGCACTGCCCGTTCGCCGACCCAACCGTAGATGGGACACCACTGGA |
| | | ACCAGGGCCGGTAAGTCCAAGCAGCCGCCGCCGTTAGCCCAAGAGCAACAA |
| | | CAGCGCCAAGGCTACCGCTCATGGCGCGGGCACAAGAACGCCATAGTTGCTT |
| | | GCTTGCAAGACTGTGGGGGCAACATCTCCTTCGCCCGCCGCTTTCTTCTCTA |
| | | CCATCACGGCGTGGCCTTCCCCCGTAACATCCTGCATTACTACCGTCATCTCT |
| | | ACAGCCCATACTGCACCGGCGGCAGCGGCAGCAACAGCAGCGGCCACACAG |
| | | AAGCAAAGGCGACCGGATAGCAAGACTCTGACAAAGCCCAAGAAATCCACAG |
| | | CGGCGGCAGCAGCAGGAGGAGGAGCGCTGCGTCTGGCGCCCAACGAACCC |
| | | GTATCGACCCGCGAGCTTAGAAACAGGATTTTTCCCACTCTGTATGCTATATTT |
| | | CAACAGAGCAGGGGCCAAGAACAAGAGCTGAAAATAAAAAACAGGTCTCTGC |
| | | GATCCCTCACCCGCAGCTGCCTGTATCACAAAAGCGAAGATCAGCTTCGGCG |
| | | CACGCTGGAAGACGCGGAGGCTCTCTTCAGTAAATACTGCGCGCTGACTCTT |
| | | AAGGACTAGTTTCGCGCCCTTTCTCAAATTTAAGCGCGAAAACTACGTCATCT |
| | | CCAGCGGCCACACCCGGCGCCAGCACCTGTTGTCAGCGCCATTATGAGCAA |
| | | GGAAATTCCCACGCCCTACATGTGGAGTTACCAGCCACAAATGGGACTTGCG |
| | | GCTGGAGCTGCCCAAGACTACTCAACCCGAATAAACTACATGAGCGCGGGAC |
| | | CCCACATGATATCCCGGGTCAACGGAATACGCGCCCACCGAAACCGAATTCT |
| | | CCTGGAACAGGCGGCTATTACCACCACACCTCGTAATAACCTTAATCCCCGTA |
| | | GTTGGCCCGCTGCCCTGGTGTACCAGGAAAGTCCCGCTCCCACCACTGTGGT |
| | | ACTTCCCAGAGACGCCCAGGCCGAAGTTCAGATGACTAACTCAGGGGCGCAG |
| | | CTTGCGGGGGGCTTTCGTCACAGGGTGCGGTCGCCCGGGCAGGGTATAACT |
| | | CACCTGACAATCAGAGGGCGAGGTATTCAGCTCAACGACGAGTCGGTGAGCT |
| | | CCTCGCTTGGTCTCCGTCCGGACGGGACATTTCAGATCGGCGGCGCCGGCC |
| | | GCTCTTCATTCACGCCTCGTCAGGCAATCCTAACTCTGCAGACCTCGTCCTCT |
| | | GAGCCGCGCTCTGGAGGCATTGGAACTCTGCAATTTATTGAGGAGTTTGTGC |
| | | CATCGGTCTACTTTAACCCCTTCTCGGGACCTCCCGGCCACTATCCGGATCAA |
| | | TTTATTCCTAACTTTGACGCGGTAAAGGACTCGGCGGACGGCTACGACTGAAT |
| | | GTTAAGTGGAGAGGCAGAGCAACTGCGCCTGAAACACCTGGTCCACTGTCGC |
| | | CGCCACAAGTGCTTTGCCCGCGACTCCGGTGAGTTTTGCTACTTTGAATTGCC |
| | | CGAGGATCATATCGAGGGCCCGGCGCACGGCGTCCGGCTTACCGCCCAGGG |
| | | AGAGCTTGCCCGTAGCCTGATTCGGGAGTTTACCCAGCGCCCCCTGCTAGTT |
| | | GAGCGGGACAGGGGACCCTGTGTTCTCACTGTGATTTGCAACTGTCCTAACC |
| | | CTGGATTACATCAAGATCCTCTAGTTAATTAACTAGAGTACCCGGGGATOTTAT |
| | | TCCCTTTAACTAATAAAAAAAAATAATAAAGCATCACTTACTTAAAATCAGTTAG |
| | | CAAATTTCTGTCCAGTTTATTCAGCAGCACCTCCTTGCCCTCCTCCCAGCTCT |
| | | GGTATTGCAGCTTCCTCCTGGCTGCAAACTTTCTCCACAATCTAAATGGAATG |
| | | TCAGTTTCCTCCTGTTCCTGTCCATCCGCACCCACTATCTTCATGTTGTTGCAG |
| | | ATGAAGCGCGCAAGACCGTCTGAAGATACCTTCAACCCCGTGTATCCATATGA |
| | | CACGGAAACCGGTCCTCCAACTGTGCCTTTTCTTACTCCTCCCTTTGTATCCC |
| | | CCAATGGGTTTCAAGAGAGTCCCCCTGGGGTACTCTCTTTGCGCCTATCCGA |
| | | ACCTCTAGTTACCTCCAATGGCATGCTTGCGCTCAAAATGGGCAACGGCCTCT |
| | | CTCTGGACGAGGCCGGCAACCTTACCTCCCAAAATGTAACCACTGTGAGCCC |
| | | ACCTCTCAAAAAAACCAAGTCAAACATAAACCTGGAAATATCTGCACCCCTCA |
| | | CAGTTACCTCAGAAGCCCTAACTGTGGCTGCCGCCGCACCTCTAATGGTCGC |
| | | GGGCAACACACTCACCATGCAATCACAGGCCCCGCTAACCGTGCACGACTCC |
| | | AAACTTAGCATTGCCACCCAAGGACCCCTCACAGTGTCAGAAGGAAAGCTAG |
| | | CCCTGCAAACATCAGGCCCCCTCACCACCACCGATAGCAGTACCCTTACTATC |
| | | ACTGCCTCACCCCCTCTAACTACTGCCACTGGTAGCTTGGGCATTGACTTGAA |
| | | AGAGCCCATTTATACACAAAATGGAAAACTAGGACTAAAGTACGGGGCTCCTT |
| | | TGCATGTAACAGACGACCTAAACACTTTGACCGTAGCAACTGGTCCAGGTGTG |
| | | ACTATTAATAATACTTCCTTGCAAACTAAAGTTACTGGAGCCTTGGGTTTTGAT |
| | | TCACAAGGCAATATGCAACTTAATGTAGCAGGAGGACTAAGGATTGATTCTCA |
| | | AAACAGACGCCTTATACTTGATGTTAGTTATCCGTTTGATGCTCAAAACCAACT |
| | | AAATCTAAGACTAGGACAGGGCCCTCTTTTTATAAACTCAGCCCACAACTTGG |
| | | ATATTAACTACAACAAAGGCCTTTACTTGTTTACAGCTTCAAACAATTCCAAAA |
| | | AGCTTGAGGTTAACCTAAGCACTGCCAAGGGGTTGATGTTTGACGCTACAGC |
| | | CATAGCCATTAATGCAGGAGATGGGCTTGAATTTGGTTCACCTAATGCACCAA |
| | | ACACAAATCCCCTCAAAACAAAAATTGGCCATGGCCTAGAATTTGATTCAAACA |
| | | AGGCTATGGTTCCTAAACTAGGAACTGGCCTTAGTTTTGACAGCACAGGTGCC |
| | | ATTACAGTAGGAAACAAAAATAATGATAAGCTAACTTTGTGGACCACACCAGC |
| | | TCCATCTCCTAACTGTAGACTAAATGCAGAGAAAGATGCTAAACTCACTTTGGT |
| | | CTTAACAAAATGTGGCAGTCAAATACTTGCTACAGTTTCAGTTTTGGCTGTTAA |
| | | AGGCAGTTTGGCTCCAATATCTGGAACAGTTCAAAGTGCTCATCTTATTATAAG |
| | | ATTTGACGAAAATGGAGTGCTACTAAACAATTCCTTCCTGGACCCAGAATATT |
| | | GGAACTTTAGAAATGGAGATCTTACTGAAGGCACAGCCTATACAAACGCTGTT |
| | | GGATTTATGCCTAACCTATCAGCTTATCCAAAATCTCACGGTAAAACTGCCAAA |
| | | AGTAACATTGTCAGTCAAGTTTACTTAAACGGAGACAAAACTAAACCTGTAACA |
| | | CTAACCATTACACTAAACGGTACACAGGAAACAGGAGACACAACTCCAAGTGC |
| | | ATACTCTATGTCATTTTCATGGGACTGGTCTGGCCACAACTACATTAATGAAAT |
| | | ATTTGCCACATCCTCTTACACTTTTTCATACATTGCCCAAGAATAAAGAATCGT |
| | | TTGTGTTATGTTTCAACGTGTTTATTTTTCAATTGCAGAAAATTTCAAGTCATTT |
| | | TTCATTCAGTAGTATAGCCCCACCACCACATAGCTTATACAGATCACCGTACC |
| | | TTAATCAAACTCACAGAACCCTAGTATTCAACCTGCCACCTCCCTOCCAACAC |
| | | ACAGAGTACACAGTCCTTTCTCCCCGGCTGGCCTTAAAAAGCATCATATCATG |
| | | GGTAACAGACATATTCTTAGGTGTTATATTCCACACGGTTTCCTGTCGAGCCA |
| | | AACGCTCATCAGTGATATTAATAAACTCCCCGGGCAGCTCACTTAAGTTCATG |
| | | TCGCTGTCCAGCTGCTGAGCCACAGGCTGCTGTCCAACTTGCGGTTGCTTAA |
| | | CGGGCGGCGAAGGAGAAGTCCACGCCTACATGGGGGTAGAGTCATAATCGT |
| | | GCATCAGGATAGGGCGGTGGTGCTGCAGCAGCGCGCGAATAAACTGCTGCC |
| | | GCCGCCGCTCCGTCCTGCAGGAATACAACATGGCAGTGGTCTCCTCAGCGAT |
| | | GATTCGCACCGCCCGCAGCATAAGGCGCCTTGTCCTCCGGGCACAGCAGCG |
| | | CACCCTGATCTCACTTAAATCAGCACAGTAACTGCAGCACAGCACCACAATAT |
| | | TGTTCAAAATCCCACAGTGCAAGGCGCTGTATCCAAAGCTCATGGGGGGAC |
| | | CACAGAACCCACGTGGCCATCATACCACAAGCGCAGGTAGATTAAGTGGCGA |
| | | CCCCTCATAAACACGCTGGACATAAACATTACCTCTTTTGGCATGTTGTAATTC |
| | | ACCACCTCCCGGTACCATATAAACCTCTGATTAAACATGGCGCCATCCACCAC |
| | | CATCCTAAACCAGCTGGCCAAAACCTGCCCGCCGGCTATACACTGCAGGGAA |
| | | CCGGGACTGGAACAATGACAGTGGAGAGCCCAGGACTCGTAACCATGGATCA |
| | | TCATGCTCGTCATGATATCAATGTTGGCACAACACAGGCACACGTGCATACAC |
| | | TTCCTCAGGATTACAAGCTCCTCCCGCGTTAGAACCATATCCCAGGGAACAAC |
| | | CCATTCCTGAATCAGCGTAAATCCCACACTGCAGGGAAGACCTCGCACGTAA |
| | | CTCACGTTGTGCATTGTCAAAGTGTTACATTCGGGCAGCAGCGGATGATCCTC |
| | | CAGTATGGTAGCGCGGGTTTCTGTCTCAAAAGGAGGTAGACGATCCCTACTG |
| | | TACGGAGTGCGCCGAGACAACCGAGATCGTGTTGGTCGTAGTGTCATGCCAA |
| | | ATGGAACGCCGGACGTAGTCATATTTCCTGAAGCAAAACCAGGTGCGGGCGT |
| | | GACAAACAGATCTGCGTCTCCGGTCTCGCCGCTTAGATCGCTCTGTGTAGTA |
| | | GTTGTAGTATATCCACTCTCTCAAAGCATCCAGGCGCCCCCTGGCTTCGGGTT |
| | | CTATGTAAACTCCTTCATGCGCCGCTGCCCTGATAACATCCACCACCGCAGAA |
| | | TAAGCCACACCCAGCCAACCTACACATTCGTTCTGCGAGTCACACACGGGAG |
| | | GAGCGGGAAGAGCTGGAAGAACCATGTTTTTTTTTTTATTCCAAAAGATTATCC |
| | | AAAACCTCAAAATGAAGATCTATTAAGTGAACGCGCTCCCCTCCGGTGGCGTG |
| | | GTCAAACTCTACAGCCAAAGAACAGATAATGGCATTTGTAAGATGTTGCACAA |
| | | TGGCTTCCAAAAGGCAAACGGCCCTCACGTCCAAGTGGACGTAAAGGCTAAA |
| | | CCCTTCAGGGTGAATCTCCTCTATAAACATTCCAGCACCTTCAACCATGCCCA |
| | | AATAATTCTCATCTCGCCACCTTCTCAATATATCTCTAAGCAAATCCCGAATAT |
| | | TAAGTCCGGCCATTGTAAAAATCTGCTCCAGAGCGCCCTCCACCTTCAGCCTC |
| | | AAGCAGCGAATCATGATTGCAAAAATTCAGGTTCCTCACAGACCTGTATAAGA |
| | | TTCAAAAGCGGAACATTAACAAAAATACCGCGATCCCGTAGGTCCCTTCGCAG |
| | | GGCCAGCTGAACATAATCGTGCAGGTCTGCACGGACCAGCGCGGCCACTTC |
| | | CCCGCCAGGAACCATGACAAAAGAACCCACACTGATTATGACACGCATACTC |
| | | GGAGCTATGCTAACCAGCGTAGCCCCGATGTAAGCTTGTTGCATGGGGGGCG |
| | | ATATAAAATGCAAGGTGCTGCTCAAAAAATCAGGCAAAGCCTCGCGCAAAAAA |
| | | GAAAGCACATCGTAGTCATGCTCATGCAGATAAAGGCAGGTAAGCTCCGGAA |
| | | CCACCACAGAAAAAGACACCATTTTTCTCTCAAACATGTCTGCGGGTTTCTGC |
| | | ATAAACACAAAATAAAATAACAAAAAAACATTTAAACATTAGAAGCCTGTCTTAC |
| | | AACAGGAAAAACAACCETTATAAGCATAAGACGGACTACGGCCATGCCGGCG |
| | | TGACCGTAAAAAAACTGGTCACCGTGATTAAAAAGCACCACCGACAGCTCCTC |
| | | GGTCATGTCCGGAGTCATAATGTAAGACTCGGTAAACACATCAGGTTGATTCA |
| | | CATCGGTCAGTGCTAAAAAGCGACCGAAATAGCCCGGGGGAATACATACCCG |
| | | CAGGCGTAGAGACAACATTACAGCCCCCATAGGAGGTATAACAAAATTAATAG |
| | | GAGAGAAAAACACATAAACACCTGAAAAACCCTCCTGCCTAGGCAAAATAGCA |
| | | CCCTCCCGCTCCAGAACAACATACAGCGCTTCCACAGCGGCAGCCATAACAG |
| | | TCAGCCTTACCAGTAAAAAAGAAAACCTATTAAAAAAACACCACTCGACACGG |
| | | CACCAGCTCAATCAGTCACAGTGTAAAAAAGGGCCAAGTGCAGAGCGAGTAT |
| | | ATATAGGACTAAAAAATGACGTAACGGTTAAAGTCCACAAAAAACACCCAGAA |
| | | AACCGCACGCGAACCTACGCCCAGAAACGAAAGCCAAAAAACCCACAACTTC |
| | | CTCAAATCGTCACTTCCGTTTTCCCACGTTACGTCACTTCCCATTTTAAGAAAA |
| | | CTACAATTCCCAACACATACAAGTTACTCCGCCCTAAAACCTACGTCACCCGC |
| | | CCCGTTCCCACGCCCCGCGCCACGTCACAAACTCCACCCCCTCATTATCATAT |
| | | TGGCTTCAATCCAAAATAATCATCAATAATATACCTTATTTTGGATTGAAGCCA |
| | | ATATGATAATGAGGGGGTGGAGTTTGTGACGTGGCGCGGGGCGTGGGAACG |
| | | GGGCGGGTGACGTAGTAGTGTGGCGGAAGTGTGATGTTGCAAGTGTGGCGG |
| | | AACACATGTAAGCGACGGATGTGGCAAAAGTGACGTTTTTGGTGTGCGCCGG |
| | | ATCCACAGGACGGGTGTGGTCGCCATGATCGCGTAGTCGATAGTGGCTCCAA |
| | | GTAGCGAAGCGAGCAGGACTGGGGGGCGGCCAAAGCGGTCGGACAGTGCT |
| | | CCGAGAACGGGTGCGCATAGAAATTGCATCAACGCATATAGCGCTAGCAGCA |
| | | CGCCATAGTGACTGGCGATGCTGTCGGAATGGACGATATCCCGCAAGAGGCC |
| | | CGGCAGTACCGGCATAACCAAGCCTATGCCTACAGCATCCAGGGTGACGGTG |
| | | CCGAGGATGACGATGAGCGCATTGTTAGATTTCATACACGGTGCCTGACTGC |
| | | GTTAGCAATTTAACTGTGATAAACTACCGCATTAAAGCTTATCGAATTOGTAAT |
| | | CATGTCATAGCTGTTTCCTGTGTGAAATTGTTATCCGCTCACAATTCCACACAA |
| | | CATACGAGCCGGAAGCATAAAGTGTAAAGCCTGGGGTGCCTAATGAGTGAGC |
| | | TAACTCACATTAATTGCGTTGCGCTCACTGCCCGCTTTCCAGTCGGGAAACCT |
| | | GTCGTGC |
| 2 | Parent B; | GGTACCCAACTCCATGCTTAACAGTCCCCAGGTACAGCCCACCCTGCGTCGC |
| | without | AACCAGGAACAGCTCTACAGCTTCCTGGAGCGCCACTCGCCCTACTTCCGCA |
| | plasmid | GCCACAGTGCGCAGATTAGGAGCGCCACTTCTTTTTGTCACTTGAAAAACATG |
| | backbone | TAAAAATAATGTACTAGGAGACACTTTCAATAAAGGCAAATGTTTTTATTTGTAC |
| | elements | ACTCTCGGGTGATTATTTACCCCCACCCTTGCCGTCTGCGCCGTTTAAAAATC |
| | (e.g., | AAAGGGGTTCTGCCGCGCATCGCTATGCGCCACTGGCAGGGACACGTTGCG |
| | antibiotic | ATACTGGTGTTTAGTGCTCCACTTAAACTCAGGCACAACCATCCGCGGCAGCT |
| | resistance | CGGTGAAGTTTTCACTCCACAGGCTGCGCACCATCACCAACGCGTTTAGCAG |
| | gene, ori) | GTCGGGCGCCGATATCTTGAAGTCGCAGTTGGGGCCTCCGCCCTGCGCGCG |
| | | CGAGTTGCGATACACAGGGTTGCAGCACTGGAACACTATCAGCGCCGGGTG |
| | | GTGCACGCTGGCCAGCACGCTCTTGTCGGAGATCAGATCCGCGTCCAGGTC |
| | | CTCCGCGTTGCTCAGGGCGAACGGAGTCAACTTTGGTAGCTGCCTTCCCAAA |
| | | AAGGGCGCGTGCCCAGGCTTTGAGTTGCACTCGCACCGTAGTGGCATCAAAA |
| | | GGTGACCGTGCCCGGTCTGGGCGTTAGGATACAGCGCCTGCATAAAAGCCTT |
| | | GATCTGCTTAAAAGCCACCTGAGCCTTTGCGCCTTCAGAGAAGAACATGCCG |
| | | CAAGACTTGCCGGAAAACTGATTGGCCGGACAGGCCGCGTCGTGCACGCAG |
| | | CACCTTGCGTCGGTGTTGGAGATCTGCACCACATTTCGGCCCCACCGGTTCT |
| | | TCACGATCTTGGCCTTGCTAGACTGCTCCTTCAGCGCGCGCTGCCCGTTTTC |
| | | GCTCGTCACATCCATTTCAATCACGTGCTCCTTATTTATCATAATGCTTCCGTG |
| | | TAGACACTTAAGCTCGCCTTCGATCTCAGCGCAGCGGTGCAGCCACAACGCG |
| | | CAGCCCGTGGGCTCGTGATGCTTGTAGGTCACCTCTGCAAACGACTGCAGGT |
| | | ACGCCTGCAGGAATCGCCCCATCATCGTCACAAAGGTCTTGTTGCTGGTGAA |
| | | GGTCAGCTGCAACCCGCGGTGCTCCTCGTTCAGCCAGGTCTTGCATACGGCC |
| | | GCCAGAGCTTCCACTTGGTCAGGCAGTAGTTTGAAGTTCGCCTTTAGATCGTT |
| | | ATCCACGTGGTACTTGTCCATCAGCGCGCGCGCAGCCTCCATGCCCTTCTCC |
| | | CACGCAGACACGATCGGCACACTCAGCGGGTTCATCACCGTAATTTCACTTTC |
| | | CGCTTCGCTGGGCTCTTCCTCTTCCTCTTGCGTCCGCATACCACGCGCCACT |
| | | GGGTCGTCTTCATTCAGCCGCCGCACTGTGCGCTTACCTCCTTTGCCATGCTT |
| | | GATTAGCACCGGTGGGTTGCTGAAACCCACCATTTGTAGCGCCACATCTTCTC |
| | | TTTCTTCCTCGCTGTCCACGATTACCTCTGGTGATGGGGGGCGCTCGGGCTT |
| | | GGGAGAAGGGCGCTTCTTTTTCTTCTTGGGCGCAATGGCCAAATCCGCCGCC |
| | | GAGGTCGATGGCCGCGGGCTGGGTGTGCGCGGCACCAGCGCGTCTTGTGAT |
| | | GAGTCTTCCTCGTCCTCGGACTCGATACGCCGCCTCATCCGCTTTTTTGGGG |
| | | GCGCCCGGGGAGGCGGCGGCGACGGGGACGGGGACGACACGTCCTCCATG |
| | | GTTGGGGGACGTCGCGCCGCACCGCGTCCGCGCTCGGGGGTGGTTTCGCG |
| | | CTGCTCCTCTTCCCGACTGGCCATTTCCTTCTCCTATAGGCAGAAAAAGATCA |
| | | TGGAGTCAGTCGAGAAGAAGGACAGCCTAACCGCCCCCTCTGAGTTCGCCAC |
| | | CACCGCCTCCACCGATGCCGCCAACGCGCCTACCACCTTCCCCGTCGAGGC |
| | | ACCCCCGCTTGAGGAGGAGGAAGTGATTATCGAGCAGGACCCAGGTTTTGTA |
| | | AGCGAAGACGACGAGGACCGCTCAGTACCAACAGAGGATAAAAAGCAAGACC |
| | | AGGACAACGCAGAGGCAAACGAGGAACAAGTCGGGGGGGGGACGAAAGG |
| | | CATGGCGACTACCTAGATGTGGGAGACGACGTGCTGTTGAAGCATCTGCAGC |
| | | GCCAGTGCGCCATTATCTGCGACGCGTTGCAAGAGCGCAGCGATGTGCCCCT |
| | | CGCCATAGCGGATGTCAGCCTTGCCTACGAACGCCACCTATTCTCACCGCGC |
| | | GTACCCCCCAAACGCCAAGAAAACGGCACATGCGAGCCCAACCCGCGCCTC |
| | | AACTTCTACCCCGTATTTGCCGTGCCAGAGGTGCTTGCCACCTATCACATCTT |
| | | TTTCCAAAACTGCAAGATACCCCTATCCTGCCGTGCCAACCGCAGCCGAGCG |
| | | GACAAGCAGCTGGCCTTGCGGCAGGGCGCTGTCATACCTGATATCGCCTCGC |
| | | TCAACGAAGTGCCAAAAATCTTTGAGGGTCTTGGACGCGACGAGAAGCGCGC |
| | | GGCAAACGCTCTGCAACAGGAAAACAGCGAAAATGAAAGTCACTCTGGAGTG |
| | | TTGGTGGAACTCGAGGGTGACAACGCGCGCCTAGCCGTACTAAAACGCAGCA |
| | | TCGAGGTCACCCACTTTGCCTACCCGGCACTTAACCTACCCCCCAAGGTCAT |
| | | GAGCACAGTCATGAGTGAGCTGATCGTGCGCCGTGCGCAGCCCCTGGAGAG |
| | | GGATGCAAATTTGCAAGAACAAACAGAGGAGGGCCTACCCGCAGTTGGCGAC |
| | | GAGCAGCTAGCGCGCTGGCTTCAAACGCGCGAGCCTGCCGACTTGGAGGAG |
| | | CGACGCAAACTAATGATGGCCGCAGTGCTCGTTACCGTGGAGCTTGAGTGCA |
| | | TGCAGCGGTTCTTTGCTGACCCGGAGATGCAGCGCAAGCTAGAGGAAACATT |
| | | GCACTACACCTTTCGACAGGGCTACGTACGCCAGGCCTGCAAGATCTCCAAC |
| | | GTGGAGCTCTGCAACCTGGTCTCCTACCTTGGAATTTTGCACGAAAACCGCCT |
| | | TGGGCAAAACGTGCTTCATTCCACGCTCAAGGGCGAGGCGCGCCGCGACTA |
| | | CGTCCGCGACTGCGTTTACTTATTTCTATGCTACACCTGGCAGACGGCCATGG |
| | | GCGTTTGGCAGCAGTGCTTGGAGGAGTGCAACCTCAAGGAGCTGCAGAAACT |
| | | GCTAAAGCAAAACTTGAAGGACCTATGGACGGCCTTCAACGAGCGCTCCGTG |
| | | GCCGCGCACCTGGCGGACATCATTTTCCCCGAACGCCTGCTTAAAACCCTGC |
| | | AACAGGGTCTGCCAGACTTCACCAGTCAAAGCATGTTGCAGAACTTTAGGAAC |
| | | TTTATCCTAGAGCGCTCAGGAATCTTGCCCGCCACCTGCTGTGCACTTCCTAG |
| | | CGACTTTGTGCCCATTAAGTACCGCGAATGCCCTCCGCCGCTTTGGGGCCAC |
| | | TGCTACCTTCTGCAGCTAGCCAACTACCTTGCCTACCACTCTGACATAATGGA |
| | | AGACGTGAGCGGTGACGGTCTACTGGAGTGTCACTGTCGCTGCAACCTATGC |
| | | ACCCCGCACCGCTCCCTGGTTTGCAATTCGCAGCTGCTTAACGAAAGTCAAAT |
| | | TATCGGTACCTTTGAGCTGCAGGGTCCCTCGCCTGACGAAAAGTCCGCGGCT |
| | | CCGGGGTTGAAACTCACTCCGGGGCTGTGGACGTCGGCTTACCTTCGCAAAT |
| | | TTGTACCTGAGGACTACCACGCCCACGAGATTAGGTTCTACGAAGACCAATCC |
| | | CGCCCGCCTAATGCGGAGCTTACCGCCTGCGTCATTACCCAGGGCCACATTC |
| | | TTGGCCAATTGCAAGCCATCAACAAAGCCCGCCAAGAGTTTCTGCTACGAAAG |
| | | GGACGGGGGGTTTACTTGGACCCCCAGTCCGGCGAGGAGCTCAACCCAATC |
| | | CCCCCGCCGCCGCAGCCCTATCAGCAGCAGCCGCGGGCCCTTGCTTCCCAG |
| | | GATGGCACCCAAAAAGAAGCTGCAGCTGCCGCCGCCACCCACGGACGAGGA |
| | | GGAATACTGGGACAGTCAGGCAGAGGAGGTTTTGGACGAGGAGGAGGAGGA |
| | | CATGATGGAAGACTGGGAGAGCCTAGACGAGGAAGCTTCCGAGGTCGAAGA |
| | | GGTGTCAGACGAAACACCGTCACCCTCGGTCGCATTCCCCTCGCCGGCGCC |
| | | CCAGAAATCGGCAACCGGTTCCAGCATGGCTACAACCTCCGCTCCTCAGGCG |
| | | CCGCCGGCACTGCCCGTTCGCCGACCCAACCGTAGATGGGACACCACTGGA |
| | | ACCAGGGCCGGTAAGTCCAAGCAGCCGCCGCCGTTAGCCCAAGAGCAACAA |
| | | CAGCGCCAAGGCTACCGCTCATGGCGCGGGCACAAGAACGCCATAGTTGCTT |
| | | GCTTGCAAGACTGTGGGGGCAACATCTCCTTCGCCCGCCGCTTTCTTCTCTA |
| | | CCATCACGGCGTGGCCTTCCCCCGTAACATCCTGCATTACTACCGTCATCTCT |
| | | ACAGCCCATACTGCACCGGCGGCAGCGGCAGCAACAGCAGCGGCCACACAG |
| | | AAGCAAAGGCGACCGGATAGCAAGACTCTGACAAAGCCCAAGAAATCCACAG |
| | | CGGCGGCAGCAGCAGGAGGAGGAGCGCTGCGTCTGGCGCCCAACGAACCC |
| | | GTATCGACCCGCGAGCTTAGAAACAGGATTTTTCCCACTCTGTATGCTATATTT |
| | | CAACAGAGCAGGGGCCAAGAACAAGAGCTGAAAATAAAAAACAGGTCTCTGC |
| | | GATCCCTCACCCGCAGCTGCCTGTATCACAAAAGCGAAGATCAGCTTCGGCG |
| | | CACGCTGGAAGACGCGGAGGCTCTCTTCAGTAAATACTGCGCGCTGACTCTT |
| | | AAGGACTAGTTTCGCGCCCTTTCTCAAATTTAAGCGCGAAAACTACGTCATCT |
| | | CCAGCGGCCACACCCGGCGCCAGCACCTGTTGTCAGCGCCATTATGAGCAA |
| | | GGAAATTCCCACGCCCTACATGTGGAGTTACCAGCCACAAATGGGACTTGCG |
| | | GCTGGAGCTGCCCAAGACTACTCAACCCGAATAAACTACATGAGCGCGGGAC |
| | | CCCACATGATATCCCGGGTCAACGGAATACGCGCCCACCGAAACCGAATTCT |
| | | CCTGGAACAGGCGGCTATTACCACCACACCTCGTAATAACCTTAATCCCCGTA |
| | | GTTGGCCCGCTGCCCTGGTGTACCAGGAAAGTCCCGCTCCCACCACTGTGGT |
| | | ACTTCCCAGAGACGCCCAGGCCGAAGTTCAGATGACTAACTCAGGGGCGCAG |
| | | CTTGCGGGGGGCTTTCGTCACAGGGTGCGGTCGCCCGGGCGTTTTAGGGCG |
| | | GAGTAACTTGTATGTGTTGGGAATTGTAGTTTTCTTAAAATGGGAAGTGACGTA |
| | | ACGTGGGAAAACGGAAGTGACGATTTGAGGAAGTTGTGGGTTTTTTGGCTTTC |
| | | GTTTCTGGGCGTAGGTTCGCGTGCGGTTTTCTGGGTGTTTTTTGTGGACTTTA |
| | | ACCGTTACGTCATTTTTTAGTCCTATATATACTCGCTCTGCACTTGGCCCTTTT |
| | | TTACACTGTGACTGATTGAGCTGGTGCCGTGTCGAGTGGTGTTTTTTTAATAG |
| | | GTTTTCTTTTTTACTGGTAAGGCTGACTGTTATGGCTGCCGCTGTGGAAGCGC |
| | | TGTATGTTGTTCTGGAGCGGGAGGGTGCTATTTTGCCTAGGCAGGAGGGTTT |
| | | TTCAGGTGTTTATGTGTTTTTCTCTCCTATTAATTTTGTTATACCTCCTATGGGG |
| | | GCTGTAATGTTGTCTCTACGCCTGCGGGTATGTATTCCCCCGGGCTATTTCGG |
| | | TCGCTTTTTAGCACTGACCGATGTGAATCAACCTGATGTGTTTACCGAGTCTTA |
| | | CATTATGACTCCGGACATGACCGAGGAGCTGTCGGTGGTGCTTTTTAATCACG |
| | | GTGACCAGTTTTTTTACGGTCACGCCGGCATGGCCGTAGTCCGTCTTATGCTT |
| | | ATAAGGGTTGTTTTTCCTGTTGTAAGACAGGCTTCTAATGTTTAAATGTTTTTTT |
| | | GTTATTTTATTTTGTGTTTATGCAGAAACCCGCAGACATGTTTGAGAGAAAAAT |
| | | GGTGTCTTTTTCTGTGGTGGTTCCGGAGCTTACCTGCCTTTATCTGCATGAGC |
| | | ATGACTACGATGTGCTTTCTTTTTTGCGCGAGGCTTTGCCTGATTTTTTGAGCA |
| | | GCACCTTGCATTTTATATCGCCGCCCATGCAACAAGCTTACATCGGGGCTACG |
| | | CTGGTTAGCATAGCTCCGAGTATGCGTGTCATAATCAGTGTGGGTTCTTTTGT |
| | | CATGGTTCCTGGCGGGGAAGTGGCCGCGCTGGTCCGTGCAGACCTGCACGA |
| | | TTATGTTCAGCTGGCCCTGCGAAGGGACCTACGGGATCGCGGTATTTTTGTTA |
| | | ATGTTCCGCTTTTGAATCTTATACAGGTCTGTGAGGAACCTGAATTTTTGCAAT |
| | | CATGATTCGCTGCTTGAGGCTGAAGGTGGAGGGCGCTCTGGAGCAGATTTTT |
| | | ACAATGGCCGGACTTAATATTCGGGATTTGCTTAGAGATATATTGAGAAGGTG |
| | | GCGAGATGAGAATTATTTGGGCATGGTTGAAGGTGCTGGAATGTTTATAGAGG |
| | | AGATTCACCCTGAAGGGTTTAGCCTTTACGTCCACTTGGACGTGAGGGCCGT |
| | | TTGCCTTTTGGAAGCCATTGTGCAACATCTTACAAATGCCATTATCTGTTCTTT |
| | | GGCTGTAGAGTTTGACCACGCCACCGGAGGGGAGCGCGTTCACTTAATAGAT |
| | | CTTCATTTTGAGGTTTTGGATAATCTTTTGGAATAAAAAAAAAAACATGGTTCTT |
| | | CCAGCTCTTCCCGCTCCTCCCGTGTGTGACTCGCAGAACGAATGTGTAGGTT |
| | | GGCTGGGTGTGGCTTATTCTGCGGTGGTGGATGTTATCAGGGCAGCGGCGC |
| | | ATGAAGGAGTTTACATAGAACCCGAAGCCAGGGGGCGCCTGGATGCTTTGAG |
| | | AGAGTGGATATACTACAACTACTACACAGAGCGATCTAAGCGGCGAGACCGG |
| | | AGACGCAGATCTGTTTGTCACGCCCGCACCTGGTTTTGCTTCAGGAAATATGA |
| | | CTACGTCCGGCGTTCCATTTGGCATGACACTACGACCAACACGATCTCGGTT |
| | | GTCTCGGCGCACTCCGTACAGTAGGGATCGTCTACCTCCTTTTGAGACAGAA |
| | | ACCCGCGCTACCATACTGGAGGATCATCCGCTGCTGCCCGAATGTAACACTT |
| | | TGACAATGCACAACGTGAGTTACGTGCGAGGTCTTCCCTGCAGTGTGGGATT |
| | | TACGCTGATTCAGGAATGGGTTGTTCCCTGGGATATGGTTCTAACGCGGGAG |
| | | GAGCTTGTAATCCTGAGGAAGTGTATGCACGTGTGCCTGTGTTGTGCCAACAT |
| | | TGATATCATGACGAGCATGATGATCCATGGTTACGAGTCCTGGGCTCTCCACT |
| | | GTCATTGTTCCAGTCCCGGTTCCCTGCAGTGTATAGCCGGGGGGCAGGTTTT |
| | | GGCCAGCTGGTTTAGGATGGTGGTGGATGGCGCCATGTTTAATCAGAGGTTT |
| | | ATATGGTACCGGGAGGTGGTGAATTACAACATGCCAAAAGAGGTAATGTTTAT |
| | | GTCCAGCGTGTTTATGAGGGGTCGCCACTTAATCTACCTGCGCTTGTGGTATG |
| | | ATGGCCACGTGGGTTCTGTGGTCCCCGCCATGAGCTTTGGATACAGCGCCTT |
| | | GCACTGTGGGATTTTGAACAATATTGTGGTGCTGTGCTGCAGTTACTGTGCTG |
| | | ATTTAAGTGAGATCAGGGTGCGCTGCTGTGCCCGGAGGACAAGGCGCCTTAT |
| | | GCTGCGGGCGGTGCGAATCATCGCTGAGGAGACCACTGCCATGTTGTATTCC |
| | | TGCAGGACGGAGCGGCGGCGGCAGCAGTTTATTCGCGCGCTGCTGCAGCAC |
| | | CACCGCCCTATCCTGATGCACGATTATGACTCTACCCCCATGTAGGCGTGGA |
| | | CTTCTCCTTCGCCGCCCGTTAAGCAACCGCAAGTTGGACAGCAGCCTGTGGC |
| | | TCAGCAGCTGGACAGCGACATGAACTTAAGTGAGCTGCCCGGGGAGTTTATT |
| | | AATATCACTGATGAGCGTTTGGCTCGACAGGAAACCGTGTGGAATATAACACC |
| | | TAAGAATATGTCTGTTACCCATGATATGATGCTTTTTAAGGCCAGCCGGGGAG |
| | | AAAGGACTGTGTACTCTGTGTGTTGGGAGGGAGGTGGCAGGTTGAATACTAG |
| | | GGTTCTGTGAGTTTGATTAAGGTACGGTGATCTGTATAAGCTATGTGGTGGTG |
| | | GGGCTATACTACTGAATGAAAAATGACTTGAAATTTTCTGCAATTGAAAAATAA |
| | | ACACGTTGAAACATAACACAAACGATTCTTTATTCTTGGGCAATGTATGAAAAA |
| | | GTGTAAGAGGATGTGGCAAATATTTCATTAATGTAGTTGTGGCCAGACCAGTC |
| | | CCATGAAAATGACATAGAGTATGCACTTGGAGTTGTGTCTCCTGTTTCCTGTG |
| | | TACCGATCGATGGATGTCGCCCCTCCTGACGCGGTAGGAGGAGGGGAGGGT |
| | | GCCCTGCATGTCTGCCGCTGCTCTTGCTCTTGCCGCTGCTGAGGAGGGGGG |
| | | CGCATCTGCCGCAGCACCGGATGCATCTGGGAAAAGCAAAAAAGGGGCTCGT |
| | | CCCTGTTTCCGGAGGAATTTGCAAGCGGGGTCTTGCATGACGGGGAGGCAAA |
| | | CCCCCGTTCGCCGCAGTCCGGCCGGTCCGAGACTCGAACCGGGGGTCCCGC |
| | | GACTCAACCCTTGGAAAATAACCCTCCGGCTACAGGGAGCGAGCCACTTAAT |
| | | GCTTTCGCTTTCCAGCCTAACCGCTTACGCTGCGCGCGGCCAGTGGCCAAAA |
| | | AAGCTAGCGCAGCAGCCGCCGCGCCTGGAAGGAAGCCAAAAGGAGCACTCC |
| | | CCCGTTGTCTGACGTCGCACACCTGGGTTCGACACGCGGGGGGTAACCGCA |
| | | TGGATCACGGCGGACGGCCGGATACGGGGCTCGAACCCCGGTCGTCCGCCA |
| | | TGATACCCTTGCGAATTTATCCACCAGACCACGGAAGAGTGCCCGCTTACAG |
| | | GCTCTCCTTTTGCACGCTAGAGCGTCAACGATTGCGCGCGCCTGACCGGCCA |
| | | GAGCGTCCCGACCATGGAGCACTTTTTGCCGCTGCGCAACATCTGGAACCGC |
| | | GTCCGCGACTTTCCGCGCGCCTCCACCACCGCCGCCGGCATCACCTGGATG |
| | | TCCAGGTACATCTACGGATGTCGACGTTTAAACCATATG |
| 3 | Fiber | ATGAAGCGCGCAAGACCGTCTGAAGATACCTTCAACCCCGTGTATCCATATGA |
| | Parent A | CACGGAAACCGGTCCTCCAACTGTGCCTTTTCTTACTCCTCCCTTTGTATCCC |
| | backbone | CCAATGGGTTTCAAGAGAGTCCCCCTGGGGTACTCTCTTTGCGCCTATCCGA |
| | | ACCTCTAGTTACCTCCAATGGCATGCTTGCGCTCAAAATGGGCAACGGCCTCT |
| | | CTCTGGACGAGGCCGGCAACCTTACCTCCCAAAATGTAACCACTGTGAGCCC |
| | | ACCTCTCAAAAAAACCAAGTCAAACATAAACCTGGAAATATCTGCACCCCTCA |
| | | CAGTTACCTCAGAAGCCCTAACTGTGGCTGCCGCCGCACCTCTAATGGTCGC |
| | | GGGCAACACACTCACCATGCAATCACAGGCCCCGCTAACCGTGCACGACTCC |
| | | AAACTTAGCATTGCCACCCAAGGACCCCTCACAGTGTCAGAAGGAAAGCTAG |
| | | CCCTGCAAACATCAGGCCCCCTCACCACCACCGATAGCAGTACCCTTACTATC |
| | | ACTGCCTCACCCCCTCTAACTACTGCCACTGGTAGCTTGGGCATTGACTTGAA |
| | | AGAGCCCATTTATACACAAAATGGAAAACTAGGACTAAAGTACGGGGCTCCTT |
| | | TGCATGTAACAGACGACCTAAACACTTTGACCGTAGCAACTGGTCCAGGTGTG |
| | | ACTATTAATAATACTTCCTTGCAAACTAAAGTTACTGGAGCCTTGGGTTTTGAT |
| | | TCACAAGGCAATATGCAACTTAATGTAGCAGGAGGACTAAGGATTGATTCTCA |
| | | AAACAGACGCCTTATACTTGATGTTAGTTATCCGTTTGATGCTCAAAACCAACT |
| | | AAATCTAAGACTAGGACAGGGCCCTCTTTTTATAAACTCAGCCCACAACTTGG |
| | | ATATTAACTACAACAAAGGCCTTTACTTGTTTACAGCTTCAAACAATTCCAAAA |
| | | AGCTTGAGGTTAACCTAAGCACTGCCAAGGGGTTGATGTTTGACGCTACAGC |
| | | CATAGCCATTAATGCAGGAGATGGGCTTGAATTTGGTTCACCTAATGCACCAA |
| | | ACACAAATCCCCTCAAAACAAAAATTGGCCATGGCCTAGAATTTGATTCAAACA |
| | | AGGCTATGGTTCCTAAACTAGGAACTGGCCTTAGTTTTGACAGCACAGGTGCC |
| | | ATTACAGTAGGAAACAAAAATAATGATAAGCTAACTTTGTGGACCACACCAGC |
| | | TCCATCTCCTAACTGTAGACTAAATGCAGAGAAAGATGCTAAACTCACTTTGGT |
| | | CTTAACAAAATGTGGCAGTCAAATACTTGCTACAGTTTCAGTTTTGGCTGTTAA |
| | | AGGCAGTTTGGCTCCAATATCTGGAACAGTTCAAAGTGCTCATCTTATTATAAG |
| | | ATTTGACGAAAATGGAGTGCTACTAAACAATTCCTTCCTGGACCCAGAATATT |
| | | GGAACTTTAGAAATGGAGATCTTACTGAAGGCACAGCCTATACAAACGCTGTT |
| | | GGATTTATGCCTAACCTATCAGCTTATCCAAAATCTCACGGTAAAACTGCCAAA |
| | | AGTAACATTGTCAGTCAAGTTTACTTAAACGGAGACAAAACTAAACCTGTAACA |
| | | CTAACCATTACACTAAACGGTACACAGGAAACAGGAGACACAACTCCAAGTGC |
| | | ATACTCTATGTCATTTTCATGGGACTGGTCTGGCCACAACTACATTAATGAAAT |
| | | ATTTGCCACATCCTCTTACACTTTTTCATACATTGCCCAAGAATAA |
| 5 | pTP | ATGGAGCACTTTTTGCCGCTGCGCAACATCTGGAACCGCGTCCGCGACTTTC |
| | Parent A | CGCGCGCCTCCACCACCGCCGCCGGCATCACCTGGATGTCCAGGTACATCTA |
| | backbone | CGGATATCATCGCCTTATGTTGGAAGATCTCGCCCCCGGAGCCCCGGCCACC |
| | | CTACGCTGGCCCCTCTACCGCCAGCCGCCGCCGCACTTTTTGGTGGGATACC |
| | | AGTACCTGGTGCGGACTTGCAACGACTACGTATTTGACTCGAGGGCTTACTC |
| | | GCGTCTCAGGTACACCGAGCTCTCGCAGCCGGGTCACCAGACCGTTAACTGG |
| | | TCCGTTATGGCCAACTGCACTTACACCATCAACACGGGCGCATACCACCGCTT |
| | | TGTGGACATGGATGACTTCCAGTCTACCCTCACGCAGGTGCAGCAGGCCATA |
| | | TTAGCCGAGCGCGTTGTCGCCGACCTAGCCCTGCTTCAGCCGATGAGGGGC |
| | | TTCGGGGTCACACGCATGGGAGGAAGAGGGCGCCACCTACGGCCAAACTCC |
| | | GCCGCCGCCGCAGCGATAGATGCAAGAGATGCAGGACAAGAGGAAGGAGAA |
| | | GAAGAAGTGCCGGTAGAAAGGCTCATGCAAGACTACTACAAAGACCTGCGCC |
| | | GATGTCAAAACGAAGCCTGGGGCATGGCCGACCGCCTGCGCATTCAGCAGG |
| | | CCGGACCCAAGGACATGGTGCTTCTGTCGAGAAGTACTAGTGGCCACGTGGG |
| | | CCGTGCACCTTAA |
| 6 | pTP Deletion | GGGATACCAGTACCTGGTGCGGACTTGCAACGACTACGTATTTGACTCGAGG |
| | 1 | GCTTACTCGCGTCTCAGGTACACCGAGCTCTCGCAGCCGGGTCACCAGACCG |
| | [pTP | TTAACTGGTCCGTTATGGCCAACTGCACTTACACCATCAACACGGGCGCATAC |
| | sequence | CACCGCTTTGTGGACATGGATGACTTCCAGTCTACCCTCACGCAGGTGCAGC |
| | deleted to | AGGCCATATTAGCCGAGCGCGTTGTCGCCGACCTAGCCCTGCTTCAGCCGAT |
| | remove the | GAGGGGCTTCGGGGTCACACGCATGGGAGGAAGAGGGCGCCACCTACGGC |
| | pTP ORF | CAAACTCCGCCGCCGCCGCAGCGATAGATGCAAGAGATGCAGGACAAGAGG |
| | sequence] | AAGGAGAAGAAGAAGTGCCGGTAGAAAGGCTCATGCAAGACTACTACAAAGA |
| | | CCTGCGCCGATGTCAAAACGAAGCCTGGGGCATGGCCGACCGCCTGCGCAT |
| | | TCAGCAGGCCGGACCCAAGGACATG |
| 7 | L1-52K | ATGCATCCGGTGCTGCGGCAGATGCGCCCCCCTCCTCAGCAGCGGCAAGAG |
| | Parent A | CAAGAGCAGCGGCAGACATGCAGGGCACCCTCCCCTCCTCCTACCGCGTCA |
| | backbone | GGAGGGGCGACATCCGCGGTTGACGCGGCAGCAGATGGTGATTACGAACCC |
| | | CCGCGGCGCCGGGCCCGGCACTACCTGGACTTGGAGGAGGGCGAGGGCCT |
| | | GGCGCGGCTAGGAGCGCCCTCTCCTGAGCGGCACCCAAGGGTGCAGCTGAA |
| | | GCGTGATACGCGTGAGGCGTACGTGCCGCGGCAGAACCTGTTTCGCGACCG |
| | | CGAGGGAGAGGAGCCCGAGGAGATGCGGGATCGAAAGTTCCACGCAGGGC |
| | | GCGAGCTGCGGCATGGCCTGAATCGCGAGCGGTTGCTGCGCGAGGAGGACT |
| | | TTGAGCCCGACGCGCGAACCGGGATTAGTCCCGCGCGCGCACACGTGGCGG |
| | | CCGCCGACCTGGTAACCGCATACGAGCAGACGGTGAACCAGGAGATTAACTT |
| | | TCAAAAAAGCTTTAACAACCACGTGCGTACGCTTGTGGCGCGCGAGGAGGTG |
| | | GCTATAGGACTGATGCATCTGTGGGACTTTGTAAGCGCGCTGGAGCAAAACC |
| | | CAAATAGCAAGCCGCTCATGGCGCAGCTGTTCCTTATAGTGCAGCACAGCAG |
| | | GGACAACGAGGCATTCAGGGATGCGCTGCTAAACATAGTAGAGCCCGAGGG |
| | | CCGCTGGCTGCTCGATTTGATAAACATCCTGCAGAGCATAGTGGTGCAGGAG |
| | | CGCAGCTTGAGCCTGGCTGACAAGGTGGCCGCCATCAACTATTCCATGCTTA |
| | | GCCTGGGCAAGTTTTACGCCCGCAAGATATACCATACCCCTTACGTTCCCATA |
| | | GACAAGGAGGTAAAGATCGAGGGGTTCTACATGCGCATGGCGCTGAAGGTG |
| | | CTTACCTTGAGCGACGACCTGGGCGTTTATCGCAACGAGCGCATCCACAAGG |
| | | CCGTGAGCGTGAGCCGGCGGCGCGAGCTCAGCGACCGCGAGCTGATGCAC |
| | | AGCCTGCAAAGGGCCCTGGCTGGCACGGGCAGCGGCGATAGAGAGGCCGA |
| | | GTCCTACTTTGACGCGGGCGCTGACCTGCGCTGGGCCCCAAGCCGACGCGC |
| | | CCTGGAGGCAGCTGGGGCCGGACCTGGGCTGGCGGTGGCACCCGCGCGCG |
| | | CTGGCAACGTCGGCGGCGTGGAGGAATATGACGAGGACGATGAGTACGAGC |
| | | CAGAGGACGGCGAGTACTAA |
| 8 | L1-52K | CTACCTGGACTTGGAGGAGGGCGAGGGCCTGGCGCGGCTAGGAGCGCCCT |
| | Deletion 1 | CTCCTGAGCGGCACCCAAGGGTGCAGCTGAAGCGTGATACGCGTGAGGCGT |
| | [L1 52K | ACGTGCCGCGGCAGAACCTGTTTCGCGACCGCGAGGGAGAGGAGCCCGAG |
| | sequence | GAGATGCGGGATCGAAAGTTCCACGCAGGGCGCGAGCTGCGGCATGGCCTG |
| | deleted to | AATCGCGAGCGGTTGCTGCGCGAGGAGGACTTTGAGCCCGACGCGCGAACC |
| | remove the | |
| | L1-52K ORF | |
| | sequence] | |
| 9 | L1-52K | CAACCACGTGCGTACGCTTGTGGCGCGCGAGGAGGTGGCTATAGGACTGAT |
| | Deletion 2 | GCATCTGTGGGACTTTGTAAGCGCGCTGGAGCAAAACCCAAATAGCAAGCCG |
| | | CTCATGGCGCAGCTGTTCCTTATAGTGCAGCACAGCAGGGACAACGAGGCAT |
| | | TCAGGGATGCGCTGCTAAACATAGTAGAGCCCGAGGGCCGCTGGCTGCTCG |
| | | ATTTGATAAACATCCTGCAGAGCATAGTGGTGCAGGAGCGCAGCTTGAGCCT |
| | | GGCTGACAAGGTGGCCGCCATCAACTATTCCATGCTTAGCCTGGGCAAGTTT |
| | | TACGCCCGCAAGATATACCATACCCCTTACGTTCCCATAGACAAGGAGGTAAA |
| | | GATCGAGGGGTTCTACATGCGCATGGCGCTGAAGGTGCTTACCTTGAGCGAC |
| | | GACCTGGGCGTTTATCGCAACGAGCGCATCCACAAGGCCGTGAGCGTGAGC |
| | | CGGCGGCGCGAGCTCAGCGACCGCGAGCTGATGCACAGCCTGCAAAGGGC |
| | | CCTGGCTGGCACGGGCAGCGGCGATAGAGAGGCCGAGTCCTACTTTGACGC |
| | | GGGCGCTGACCTGCGCTGGGCCCCAAGCCGACGCGCCCTGGAGGCAGCTG |
| | | GGGCCGGACCTGGGCTGGCGGTGGCACCCGCGCGCGCTGGCAACGTCGGC |
| | | GGCGTGGAGGAATATGACGAGGACGATGAGTACGAGCCAGAGGACGGCGAG |
| | | TACTAAGCGGTGATGTTTCTGATCAGATGATGCAAGACGCAACGGACCCGGC |
| | | GGTGCGGGCGGCGCTGCAGAGCCAGCCGTCCGGCCTTAACTCCACGGACGA |
| | | CTGGCGCCAGGTCATGGACCGCATCATGTCGCTGACTGCGCGCAATCCTGAC |
| | | GCGTTCCGGCAGCAGCCGCAGGCCAACCGGCTCTCCGCAATTCTGGAAGCG |
| | | GTGGTCCCGGCGCGCGCAAACCCCACGCACGAGAAGGTGCTGGCGATCGTA |
| | | AACGCGCTGGCCGAAAACAGGGCCATCCGGCCCGACGAGGCCGGCCTGGTC |
| | | TACGACGCGCTGCTTCAGCGCGTGGCTCGTTACAACAGCGGCAACGTGCAGA |
| | | CCAACCTGGACCGGCTGGTGGGGGATGTGCGCGAGGCCGTGGCGCAGCGT |
| | | GAGCGCGCGCAGCAGCAGGGCAACCTGGGCTCCATGGTTGCACTAAACGCC |
| | | TTCCTGAGTACACAGCCCGCCAACGTGCCGCGGGGACAGGAGGACTACACC |
| | | AACTTTGTGAGCGCACTGCGGCTAATGGTGACTGAGACACCGCAAAGTGAGG |
| | | TGTACCAGTCTGGGCCAGACTATTTTTTCCAGACCAGTAGACAAGGCCTGCAG |
| | | ACCGTAAACCTGAGCCAGGCTTTCAAAAACTTGCAGGGGCTGTGGGGGGTGC |
| | | GGGCTCCCACAGGCGACCGCGCGACCGTGTCTAGCTTGCTGACGCCCAACT |
| | | CGCGCCTGTTGCTGCTGCTAATAGCGCCCTTCACGGACAGTGGCAGCGTGTC |
| | | CCGGGACACATACCTAGGTCACTTGCTGACACTGTACCGCGAGGCCATAGGT |
| | | CAGGCGCATGTGGACGAGCATACTTTCCAGGAGATTACAAGTGTCAGCCGCG |
| | | CGCTGGGGCAGGAGGACACGGGCAGCCTGGAGGCAACCCTAAACTACCTGC |
| | | TGACCAACCGGCGGCAGAAGATCCCCTCGTTGCACAGTTTGCACCCTTTGGC |
| | | GCATCCCATTCTCCAGTAACTTTATGTCCATGGGCGCACTCACAGACCTGGGC |
| | | CAAAACCTTCTCTACGCCAACTCCGCCCACGCGCTAGACATGACTTTTGAGGT |
| | | GGATCCCATGGACGAGCCCACCCTTCTTTATGTTTTGTTTGAAGTCTTTGACG |
| | | TGGTCCGTGTGCACCAGCCGCACCGCGGCGTCATCGAAACCGTGTACCTGC |
| | | GCACGCCCTTCTCGGCCGGCAACGCCACAACATAAAGAAGCAAGCAACATCA |
| | | ACAACAGCTGCCGCCATGGGCTCCAGTGAGCAGGAACTGAAAGCCATTGTCA |
| | | AAGATCTTGGTTGTGGGCCATATTTTTTGGGCACCTATGACAAGCGCTTTCCA |
| | | GGCTTTGTTTCTCCACACAAGCTCGCCTGCGCCATAGTCAATACGGCCGGTC |
| | | GCGAGACTGGGGGCGTACACTGGATGGCCTTTGCCTGGAACCCGCACTCAA |
| | | AAACATGCTACCTCTTTGAGCCCTTTGGCTTTTCTGACCAGCGACTCAAGCAG |
| | | GTTTACCAGTTTGAGTACGAGTCACTCCTGCGCCGTAGCGCCATTGCTTCTTC |
| | | CCCCGACCGCTGTATAACGCTGGAAAAGTCCACCCAAAGCGTACAGGGGCCC |
| | | AACTCGGCCGCCTGTGGACTATTCTGCTGCATGTTTCTCCACGCCTTTGCCAA |
| | | CTGGCCCCAAACTCCCATGGATCACAACCCCACCATGAACCTTATTACCGG |
| 10 | 100K | ATGCCCTTCTCCCACGCAGACACGATCGGCACACTCAGCGGGTTCATCACCG |
| | Parent A | TAATTTCACTTTCCGCTTCGCTGGGCTCTTCCTCTTCCTCTTGCGTCCGCATAC |
| | backbone | CACGCGCCACTGGGTCGTCTTCATTCAGCCGCCGCACTGTGCGCTTACCTCC |
| | | TTTGCCATGCTTGATTAGCACCGGTGGGTTGCTGAAACCCACCATTTGTAGCG |
| | | CCACATCTTCTCTTTCTTCCTCGCTGTCCACGATTACCTCTGGTGATGGGGGG |
| | | CGCTCGGGCTTGGGAGAAGGGCGCTTCTTTTTCTTCTTGGGCGCAATGGCCA |
| | | AATCCGCCGCCGAGGTCGATGGCCGCGGGCTGGGTGTGCGCGGCACCAGC |
| | | GCGTCTTGTGATGAGTCTTCCTCGTCCTCGGACTCGATACGCCGCCTCATCC |
| | | GCTTTTTTGGGGGCGCCCGGGGAGGCGGCGGCGACGGGGACGGGGACGAC |
| | | ACGTCCTCCATGGTTGGGGGACGTCGCGCCGCACCGCGTCCGCGCTCGGGG |
| | | GTGGTTTCGCGCTGCTCCTCTTCCCGACTGGCCATTTCCTTCTCCTATAGGCA |
| | | GAAAAAGATCATGGAGTCAGTCGAGAAGAAGGACAGCCTAACCGCCCCCTCT |
| | | GAGTTCGCCACCACCGCCTCCACCGATGCCGCCAACGCGCCTACCACCTTCC |
| | | CCGTCGAGGCACCCCCGCTTGAGGAGGAGGAAGTGATTATCGAGCAGGACC |
| | | CAGGTTTTGTAAGCGAAGACGACGAGGACCGCTCAGTACCAACAGAGGATAA |
| | | AAAGCAAGACCAGGACAACGCAGAGGCAAACGAGGAACAAGTCGGGGGGGG |
| | | GGACGAAAGGCATGGCGACTACCTAGATGTGGGAGACGACGTGCTGTTGAA |
| | | GCATCTGCAGCGCCAGTGCGCCATTATCTGCGACGCGTTGCAAGAGCGCAG |
| | | CGATGTGCCCCTCGCCATAGCGGATGTCAGCCTTGCCTACGAACGCCACCTA |
| | | TTCTCACCGCGCGTACCCCCCAAACGCCAAGAAAACGGCACATGCGAGCCCA |
| | | ACCCGCGCCTCAACTTCTACCCCGTATTTGCCGTGCCAGAGGTGCTTGCCAC |
| | | CTATCACATCTTTTTCCAAAACTGCAAGATACCCCTATCCTGCCGTGCCAACC |
| | | GCAGCCGAGCGGACAAGCAGCTGGCCTTGCGGCAGGGCGCTGTCATACCTG |
| | | ATATCGCCTCGCTCAACGAAGTGCCAAAAATCTTTGAGGGTCTTGGACGCGA |
| | | CGAGAAGCGCGCGGCAAACGCTCTGCAACAGGAAAACAGCGAAAATGAAAGT |
| | | CACTCTGGAGTGTTGGTGGAACTCGAGGGTGACAACGCGCGCCTAGCCGTA |
| | | CTAAAACGCAGCATCGAGGTCACCCACTTTGCCTACCCGGCACTTAACCTACC |
| | | CCCCAAGGTCATGAGCACAGTCATGAGTGAGCTGATCGTGCGCCGTGCGCA |
| | | GCCCCTGGAGAGGGATGCAAATTTGCAAGAACAAACAGAGGAGGGCCTACCC |
| | | GCAGTTGGCGACGAGCAGCTAGCGCGCTGGCTTCAAACGCGCGAGCCTGCC |
| | | GACTTGGAGGAGCGACGCAAACTAATGATGGCCGCAGTGCTCGTTACCGTGG |
| | | AGCTTGAGTGCATGCAGCGGTTCTTTGCTGACCCGGAGATGCAGCGCAAGCT |
| | | AGAGGAAACATTGCACTACACCTTTCGACAGGGCTACGTACGCCAGGCCTGC |
| | | AAGATCTCCAACGTGGAGCTCTGCAACCTGGTCTCCTACCTTGGAATTTTGCA |
| | | CGAAAACCGCCTTGGGCAAAACGTGCTTCATTCCACGCTCAAGGGCGAGGCG |
| | | CGCCGCGACTACGTCCGCGACTGCGTTTACTTATTTCTATGCTACACCTGGCA |
| | | GACGGCCATGGGCGTTTGGCAGCAGTGCTTGGAGGAGTGCAACCTCAAGGA |
| | | GCTGCAGAAACTGCTAAAGCAAAACTTGAAGGACCTATGGACGGCCTTCAAC |
| | | GAGCGCTCCGTGGCCGCGCACCTGGCGGACATCATTTTCCCCGAACGCCTG |
| | | CTTAAAACCCTGCAACAGGGTCTGCCAGACTTCACCAGTCAAAGCATGTTGCA |
| | | GAACTTTAGGAACTTTATCCTAGAGCGCTCAGGAATCTTGCCCGCCACCTGCT |
| | | GTGCACTTCCTAGCGACTTTGTGCCCATTAAGTACCGCGAATGCCCTCCGCC |
| | | GCTTTGGGGCCACTGCTACCTTCTGCAGCTAGCCAACTACCTTGCCTACCACT |
| | | CTGACATAATGGAAGACGTGAGCGGTGACGGTCTACTGGAGTGTCACTGTCG |
| | | CTGCAACCTATGCACCCCGCACCGCTCCCTGGTTTGCAATTCGCAGCTGCTT |
| | | AACGAAAGTCAAATTATCGGTACCTTTGAGCTGCAGGGTCCCTCGCCTGACG |
| | | AAAAGTCCGCGGCTCCGGGGTTGAAACTCACTCCGGGGCTGTGGACGTCGG |
| | | CTTACCTTCGCAAATTTGTACCTGAGGACTACCACGCCCACGAGATTAGGTTC |
| | | TACGAAGACCAATCCCGCCCGCCTAATGCGGAGCTTACCGCCTGCGTCATTA |
| | | CCCAGGGCCACATTCTTGGCCAATTGCAAGCCATCAACAAAGCCCGCCAAGA |
| | | GTTTCTGCTACGAAAGGGACGGGGGGTTTACTTGGACCCCCAGTCCGGCGA |
| | | GGAGCTCAACCCAATCCCCCCGCCGCCGCAGCCCTATCAGCAGCAGCCGCG |
| | | GGCCCTTGCTTCCCAGGATGGCACCCAAAAAGAAGCTGCAGCTGCCGCCGC |
| | | CACCCACGGACGAGGAGGAATACTGGGACAGTCAGGCAGAGGAGGTTTTGG |
| | | ACGAGGAGGAGGAGGACATGATGGAAGACTGGGAGAGCCTAGACGAGGAAG |
| | | CTTCCGAGGTCGAAGAGGTGTCAGACGAAACACCGTCACCCTCGGTCGCATT |
| | | CCCCTCGCCGGCGCCCCAGAAATCGGCAACCGGTTCCAGCATGGCTACAAC |
| | | CTCCGCTCCTCAGGCGCCGCCGGCACTGCCCGTTCGCCGACCCAACCGTAG |
| 11 | Hexon- | ATGGAGTCAGTCGAGAAGAAGGACAGCCTAACCGCCCCCTCTGAGTTCGCCA |
| | Assembly | CCACCGCCTCCACCGATGCCGCCAACGCGCCTACCACCTTCCCCGTCGAGG |
| | | CACCCCCGCTTGAGGAGGAGGAAGTGATTATCGAGCAGGACCCAGGTTTTGT |
| | | AAGCGAAGACGACGAGGACCGCTCAGTACCAACAGAGGATAAAAAGCAAGAC |
| | | CAGGACAACGCAGAGGCAAACGAGGAACAAGTCGGGGGGGGGACGAAAG |
| | | GCATGGCGACTACCTAGATGTGGGAGACGACGTGCTGTTGAAGCATCTGCAG |
| | | CGCCAGTGCGCCATTATCTGCGACGCGTTGCAAGAGCGCAGCGATGTGCCC |
| | | CTCGCCATAGCGGATGTCAGCCTTGCCTACGAACGCCACCTATTCTCACCGC |
| | | GCGTACCCCCCAAACGCCAAGAAAACGGCACATGCGAGCCCAACCCGCGCC |
| | | TCAACTTCTACCCCGTATTTGCCGTGCCAGAGGTGCTTGCCACCTATCACATC |
| | | TTTTTCCAAAACTGCAAGATACCCCTATCCTGCCGTGCCAACCGCAGCCGAGC |
| | | GGACAAGCAGCTGGCCTTGCGGCAGGGCGCTGTCATACCTGATATCGCCTC |
| | | GCTCAACGAAGTGCCAAAAATCTTTGAGGGTCTTGGACGCGACGAGAAGCGC |
| | | GCGGCAAACGCTCTGCAACAGGAAAACAGCGAAAATGAAAGTCACTCTGGAG |
| | | TGTTGGTGGAACTCGAGGGTGACAACGCGCGCCTAGCCGTACTAAAACGCAG |
| | | CATCGAGGTCACCCACTTTGCCTACCCGGCACTTAACCTACCCCCCAAGGTC |
| | | ATGAGCACAGTCATGAGTGAGCTGATCGTGCGCCGTGCGCAGCCCCTGGAG |
| | | AGGGATGCAAATTTGCAAGAACAAACAGAGGAGGGCCTACCCGCAGTTGGCG |
| | | ACGAGCAGCTAGCGCGCTGGCTTCAAACGCGCGAGCCTGCCGACTTGGAGG |
| | | AGCGACGCAAACTAATGATGGCCGCAGTGCTCGTTACCGTGGAGCTTGAGTG |
| | | CATGCAGCGGTTCTTTGCTGACCCGGAGATGCAGCGCAAGCTAGAGGAAACA |
| | | TTGCACTACACCTTTCGACAGGGCTACGTACGCCAGGCCTGCAAGATCTCCA |
| | | ACGTGGAGCTCTGCAACCTGGTCTCCTACCTTGGAATTTTGCACGAAAACCGC |
| | | CTTGGGCAAAACGTGCTTCATTCCACGCTCAAGGGCGAGGCGCGCCGCGAC |
| | | TACGTCCGCGACTGCGTTTACTTATTTCTATGCTACACCTGGCAGACGGCCAT |
| | | GGGCGTTTGGCAGCAGTGCTTGGAGGAGTGCAACCTCAAGGAGCTGCAGAA |
| | | ACTGCTAAAGCAAAACTTGAAGGACCTATGGACGGCCTTCAACGAGCGCTCC |
| | | GTGGCCGCGCACCTGGCGGACATCATTTTCCCCGAACGCCTGCTTAAAACCC |
| | | TGCAACAGGGTCTGCCAGACTTCACCAGTCAAAGCATGTTGCAGAACTTTAGG |
| | | AACTTTATCCTAGAGCGCTCAGGAATCTTGCCCGCCACCTGCTGTGCACTTCC |
| | | TAGCGACTTTGTGCCCATTAAGTACCGCGAATGCCCTCCGCCGCTTTGGGGC |
| | | CACTGCTACCTTCTGCAGCTAGCCAACTACCTTGCCTACCACTCTGACATAAT |
| | | GGAAGACGTGAGCGGTGACGGTCTACTGGAGTGTCACTGTCGCTGCAACCTA |
| | | TGCACCCCGCACCGCTCCCTGGTTTGCAATTCGCAGCTGCTTAACGAAAGTC |
| | | AAATTATCGGTACCTTTGAGCTGCAGGGTCCCTCGCCTGACGAAAAGTCCGC |
| | | GGCTCCGGGGTTGAAACTCACTCCGGGGCTGTGGACGTCGGCTTACCTTCG |
| | | CAAATTTGTACCTGAGGACTACCACGCCCACGAGATTAGGTTCTACGAAGACC |
| | | AATCCCGCCCGCCTAATGCGGAGCTTACCGCCTGCGTCATTACCCAGGGCCA |
| | | CATTCTTGGCCAATTGCAAGCCATCAACAAAGCCCGCCAAGAGTTTCTGCTAC |
| | | GAAAGGGACGGGGGGTTTACTTGGACCCCCAGTCCGGCGAGGAGCTCAACC |
| | | CAATCCCCCCGCCGCCGCAGCCCTATCAGCAGCAGCCGCGGGCCCTTGCTT |
| | | CCCAGGATGGCACCCAAAAAGAAGCTGCAGCTGCCGCCGCCACCCACGGAC |
| | | GAGGAGGAATACTGGGACAGTCAGGCAGAGGAGGTTTTGGACGAGGAGGAG |
| | | GAGGACATGATGGAAGACTGGGAGAGCCTAGACGAGGAAGCTTCCGAGGTC |
| | | GAAGAGGTGTCAGACGAAACACCGTCACCCTCGGTCGCATTCCCCTCGCCGG |
| | | CGCCCCAGAAATCGGCAACCGGTTCCAGCATGGCTACAACCTCCGCTCCTCA |
| | | GGCGCCGCCGGCACTGCCCGTTCGCCGACCCAACCGTAG |
| 12 | Sequence | TAATAA |
| | insert for | |
| | c.933insTAA | |
| | TAA | |
| | mutation of | |
| | Hexon | |
| | assembly | |
| | (inside 100K) | |
| 13 | Hexon- | ATGAGCAAGGAAATTCCCACGCCCTACATGTGGAGTTACCAGCCACAAATGG |
| | associated | GACTTGCGGCTGGAGCTGCCCAAGACTACTCAACCCGAATAAACTACATGAG |
| | precursor/ | CGCGGGACCCCACATGATATCCCGGGTCAACGGAATACGCGCCCACCGAAA |
| | pVIII | CCGAATTCTCCTGGAACAGGCGGCTATTACCACCACACCTCGTAATAACCTTA |
| | Parent A | ATCCCCGTAGTTGGCCCGCTGCCCTGGTGTACCAGGAAAGTCCCGCTCCCAC |
| | backbone | CACTGTGGTACTTCCCAGAGACGCCCAGGCCGAAGTTCAGATGACTAACTCA |
| | | GGGGCGCAGCTTGCGGGGGGCTTTCGTCACAGGGTGCGGTCGCCCGGGCA |
| | | GGGTATAACTCACCTGACAATCAGAGGGCGAGGTATTCAGCTCAACGACGAG |
| | | TCGGTGAGCTCCTCGCTTGGTCTCCGTCCGGACGGGACATTTCAGATCGGCG |
| | | GCGCCGGCCGCTCTTCATTCACGCCTCGTCAGGCAATCCTAACTCTGCAGAC |
| | | CTCGTCCTCTGAGCCGCGCTCTGGAGGCATTGGAACTCTGCAATTTATTGAG |
| | | GAGTTTGTGCCATCGGTCTACTTTAACCCCTTCTCGGGACCTCCCGGCCACTA |
| | | TCCGGATCAATTTATTCCTAACTTTGACGCGGTAAAGGACTCGGCGGACGGCT |
| | | ACGACTGA |
| 14 | Hexon- | ATGAGCAAGGAAATTCCCACGCCCTACATGTGGAGTTACCAGCCACAAATGG |
| | associated | GACTTGCGGCTGGAGCTGCCCAAGACTACTCAACCCGAATAAACTACATGAG |
| | precursor/ | CGCGGGACCCCACATGATATCCCGGGTCAACGGAATACGCGCCCACCGAAA |
| | pVIII | CCGAATTCTCCTGGAACAGGCGGCTATTACCACCACACCTCGTAATAACCTTA |
| | Deletion 1 | ATCCCCGTAGTTGGCCCGCTGCCCTGGTGTACCAGGAAAGTCCCGCTCCCAC |
| | [Removes | CACTGTGGTACTTCCCAGAGACGCCCAGGCCGAAGTTCAGATGACTAACTCA |
| | Hexon- | GGGGCGCAGCTTGCGGGGGGCTTTCGTCACAGGGTGCGGTC |
| | associated | |
| | precursor/ | |
| | pVIII | |
| | sequence | |
| 15 | E4ORF1 | ATGGCTGCCGCTGTGGAAGCGCTGTATGTTGTTCTGGAGCGGGAGGGTGCTA |
| | Parent A | TTTTGCCTAGGCAGGAGGGTTTTTCAGGTGTTTATGTGTTTTTCTCTCCTATTA |
| | backbone | ATTTTGTTATACCTCCTATGGGGGCTGTAATGTTGTCTCTACGCCTGCGGGTA |
| | | TGTATTCCCCCGGGCTATTTCGGTCGCTTTTTAGCACTGACCGATGTGAATCA |
| | | ACCTGATGTGTTTACCGAGTCTTACATTATGACTCCGGACATGACCGAGGAGC |
| | | TGTCGGTGGTGCTTTTTAATCACGGTGACCAGTTTTTTTACGGTCACGCCGGC |
| | | ATGGCCGTAGTCCGTCTTATGCTTATAAGGGTTGTTTTTCCTGTTGTAAGACA |
| | | GGCTTCTAATGTTTAA |
| 16 | E4ORF2 | ATGTTTGAGAGAAAAATGGTGTCTTTTTCTGTGGTGGTTCCGGAGCTTACCTG |
| | Parent A | CCTTTATCTGCATGAGCATGACTACGATGTGCTTTCTTTTTTGCGCGAGGCTTT |
| | backbone | GCCTGATTTTTTGAGCAGCACCTTGCATTTTATATCGCCGCCCATGCAACAAG |
| | | CTTACATCGGGGCTACGCTGGTTAGCATAGCTCCGAGTATGCGTGTCATAATC |
| | | AGTGTGGGTTCTTTTGTCATGGTTCCTGGCGGGGAAGTGGCCGCGCTGGTCC |
| | | GTGCAGACCTGCACGATTATGTTCAGCTGGCCCTGCGAAGGGACCTACGGGA |
| | | TCGCGGTATTTTTGTTAATGTTCCGCTTTTGAATCTTATACAGGTCTGTGAGGA |
| | | ACCTGAATTTTTGCAATCATGA |
| 17 | E4ORF3 | ATGATTCGCTGCTTGAGGCTGAAGGTGGAGGGCGCTCTGGAGCAGATTTTTA |
| | Parent A | CAATGGCCGGACTTAATATTCGGGATTTGCTTAGAGATATATTGAGAAGGTGG |
| | backbone | CGAGATGAGAATTATTTGGGCATGGTTGAAGGTGCTGGAATGTTTATAGAGGA |
| | | GATTCACCCTGAAGGGTTTAGCCTTTACGTCCACTTGGACGTGAGGGCCGTTT |
| | | GCCTTTTGGAAGCCATTGTGCAACATCTTACAAATGCCATTATCTGTTCTTTGG |
| | | CTGTAGAGTTTGACCACGCCACCGGAGGGGAGCGCGTTCACTTAATAGATCT |
| | | TCATTTTGAGGTTTTGGATAATCTTTTGGAATAA |
| 18 | E4ORF4 | ATGGTTCTTCCAGCTCTTCCCGCTCCTCCCGTGTGTGACTCGCAGAACGAATG |
| | Parent A | TGTAGGTTGGCTGGGTGTGGCTTATTCTGCGGTGGTGGATGTTATCAGGGCA |
| | backbone | GCGGCGCATGAAGGAGTTTACATAGAACCCGAAGCCAGGGGGCGCCTGGAT |
| | | GCTTTGAGAGAGTGGATATACTACAACTACTACACAGAGCGATCTAAGCGGC |
| | | GAGACCGGAGACGCAGATCTGTTTGTCACGCCCGCACCTGGTTTTGCTTCAG |
| | | GAAATATGACTACGTCCGGCGTTCCATTTGGCATGACACTACGACCAACACGA |
| | | TCTCGGTTGTCTCGGCGCACTCCGTACAGTAG |
| 19 | Ad5 ITR | AATCATCAATAATATACCTTATTTTGGATTGAAGCCAATATGATAATGAGGGGG |
| | Parent A | TGGAGTTTGTGACGTGGCGCGGGGCGTGGGAACGGGGGGGGTGACGTAG |
| | backbone | |
| 20 | Ad5 ITR | CACCCGCCCCGTTCCCACGCCCCGCGCCACGTCACAAACTCCACCCCCTCAT |
| | Deletion 1 | TATCATATTGGCTTCAATCCAAAATAATCATCAATAATATACCTTATTTTGGATT |
| | [Deletion of | GAAGCCAATATGATAATGAGGGGGTGGAGTTTGTGACGTGGCGCGGGGCGT |
| | corresponding | GGGAACGGGGGGGGTGACGTAGTAGTGTGGCGGAAGTGTGATGTTGCAAGT |
| | sequence | GTGGCGGAACACATGTAAGC |
| | to remove | |
| | Ad5 ITR | |
| | sequence] | |
| 21 | Ad5 ITR | CACCCGCCCCGTTCCCACGCCCCGCGCCACGTCACAAACTCCACCCCCTCAT |
| | Deletion 2 | TATCATATTGGCTTCAATCCAAAATAATCATCAATAATATACCTTATTTTGGATT |
| | [Deletion of | GAAGCCAATATGATAATGAGGGGGTGGAGTTTGTGACGTGGCGCGGGGCGT |
| | corresponding | GGGAACGGGGGGGGTGACGTAGTAGTGTGGCGGAAGTGTGATGTTGCAAGT |
| | sequence | GTGGCGGAACACATGTAAGCGACGGATGTGGCAAAAGTGACGTTTTTGGTGT |
| | to remove | GCGCCGGATCCACAGGACGGGTGTGGTCGCCATGATCGCGTAGTCGATAGT |
| | Ad5 ITR | GGCTCCAAGTAGCGAAGCGAGCAGGACTGGGGGGCGGCCAAAGCGGTCGG |
| | sequence | ACAGTGCTCCGAGAACGGGTGCGCATAGAAATTGCATCAACGCATATAGCGC |
| | (alternate | TAGCAGCACGCCATAGTGACTGGCGATGCTGTCGGAATGGACGATATCCCGC |
| | sequence | AAGAGGCCCGGCAGTACCGGCATAACCAAGCCTATGCCTACAGCATCCAGGG |
| | deletion)] | TGACGGTGCCGAGGATGACGATGAGCGCATTGTTAGATTTCATACACGGTGC |
| | | CTGACTGCGTTAGCAATTTAACTGTGATAAACTACCGCATTAAAGCTTATCGAA |
| | | TT |
| 22 | Fiber | GGTATAACTCACCTGACAATCAGAGGGCGAGGTATTCAGCTCAACGACGAGT |
| | Deletion 1 | CGGTGAGCTCCTCGCTTGGTCTCCGTCCGGACGGGACATTTCAGATCGGCG |
| | [Deleted | GCGCCGGCCGCTCTTCATTCACGCCTCGTCAGGCAATCCTAACTCTGCAGAC |
| | sequence | CTCGTCCTCTGAGCCGCGCTCTGGAGGCATTGGAACTCTGCAATTTATTGAG |
| | starting 5′ | GAGTTTGTGCCATCGGTCTACTTTAACCCCTTCTCGGGACCTCCCGGCCACTA |
| | to Fiber | TCCGGATCAATTTATTCCTAACTTTGACGCGGTAAAGGACTCGGCGGACGGCT |
| | gene | ACGACTGAATGTTAAGTGGAGAGGCAGAGCAACTGCGCCTGAAACACCTGGT |
| | (3′region of | CCACTGTCGCCGCCACAAGTGCTTTGCCCGCGACTCCGGTGAGTTTTGCTAC |
| | Hexon- | TTTGAATTGCCCGAGGATCATATCGAGGGCCCGGCGCACGGCGTCCGGOTTA |
| | associated | CCGCCCAGGGAGAGCTTGCCCGTAGCCTGATTCGGGAGTTTACCCAGCGCC |
| | precursor/ | CCCTGCTAGTTGAGCGGGACAGGGGACCCTGTGTTCTCACTGTGATTTGCAA |
| | pVIII), | CTGTCCTAACCCTGGATTACATCAAGATCCTCTAGTTAATTAACTAGAGTACCC |
| | encompassing | GGGGATOTTATTCCCTTTAACTAATAAAAAAAAATAATAAAGCATCACTTACTTA |
| | the entire | AAATCAGTTAGCAAATTTCTGTCCAGTTTATTCAGCAGCACCTCCTTGCCCTCC |
| | Fiber ORF] | TCCCAGCTCTGGTATTGCAGCTTCCTCCTGGCTGCAAACTTTCTCCACAATCT |
| | | AAATGGAATGTCAGTTTCCTCCTGTTCCTGTCCATCCGCACCCACTATCTTCAT |
| | | GTTGTTGCAGATGAAGCGCGCAAGACCGTCTGAAGATACCTTCAACCCCGTG |
| | | TATCCATATGACACGGAAACCGGTCCTCCAACTGTGCCTTTTCTTACTCCTCC |
| | | CTTTGTATCCCCCAATGGGTTTCAAGAGAGTCCCCCTGGGGTACTCTCTTTGC |
| | | GCCTATCCGAACCTCTAGTTACCTCCAATGGCATGCTTGCGCTCAAAATGGGC |
| | | AACGGCCTCTCTCTGGACGAGGCCGGCAACCTTACCTCCCAAAATGTAACCA |
| | | CTGTGAGCCCACCTCTCAAAAAAACCAAGTCAAACATAAACCTGGAAATATCT |
| | | GCACCCCTCACAGTTACCTCAGAAGCCCTAACTGTGGCTGCCGCCGCACCTC |
| | | TAATGGTCGCGGGCAACACACTCACCATGCAATCACAGGCCCCGCTAACCGT |
| | | GCACGACTCCAAACTTAGCATTGCCACCCAAGGACCCCTCACAGTGTCAGAA |
| | | GGAAAGCTAGCCCTGCAAACATCAGGCCCCCTCACCACCACCGATAGCAGTA |
| | | CCCTTACTATCACTGCCTCACCCCCTCTAACTACTGCCACTGGTAGCTTGGGC |
| | | ATTGACTTGAAAGAGCCCATTTATACACAAAATGGAAAACTAGGACTAAAGTAC |
| | | GGGGCTCCTTTGCATGTAACAGACGACCTAAACACTTTGACCGTAGCAACTG |
| | | GTCCAGGTGTGACTATTAATAATACTTCCTTGCAAACTAAAGTTACTGGAGCCT |
| | | TGGGTTTTGATTCACAAGGCAATATGCAACTTAATGTAGCAGGAGGACTAAGG |
| | | ATTGATTCTCAAAACAGACGCCTTATACTTGATGTTAGTTATCCGTTTGATGCT |
| | | CAAAACCAACTAAATCTAAGACTAGGACAGGGCCCTCTTTTTATAAACTCAGC |
| | | CCACAACTTGGATATTAACTACAACAAAGGCCTTTACTTGTTTACAGCTTCAAA |
| | | CAATTCCAAAAAGCTTGAGGTTAACCTAAGCACTGCCAAGGGGTTGATGTTTG |
| | | ACGCTACAGCCATAGCCATTAATGCAGGAGATGGGCTTGAATTTGGTTCACCT |
| | | AATGCACCAAACACAAATCCCCTCAAAACAAAAATTGGCCATGGCCTAGAATT |
| | | TGATTCAAACAAGGCTATGGTTCCTAAACTAGGAACTGGCCTTAGTTTTGACA |
| | | GCACAGGTGCCATTACAGTAGGAAACAAAAATAATGATAAGCTAACTTTGTGG |
| | | ACCACACCAGCTCCATCTCCTAACTGTAGACTAAATGCAGAGAAAGATGCTAA |
| | | ACTCACTTTGGTCTTAACAAAATGTGGCAGTCAAATACTTGCTACAGTTTCAGT |
| | | TTTGGCTGTTAAAGGCAGTTTGGCTCCAATATCTGGAACAGTTCAAAGTGCTC |
| | | ATCTTATTATAAGATTTGACGAAAATGGAGTGCTACTAAACAATTCCTTCCTGG |
| | | ACCCAGAATATTGGAACTTTAGAAATGGAGATCTTACTGAAGGCACAGCCTAT |
| | | ACAAACGCTGTTGGATTTATGCCTAACCTATCAGCTTATCCAAAATCTCACGGT |
| | | AAAACTGCCAAAAGTAACATTGTCAGTCAAGTTTACTTAAACGGAGACAAAACT |
| | | AAACCTGTAACACTAACCATTACACTAAACGGTACACAGGAAACAGGAGACAC |
| | | AACTCCAAGTGCATACTCTATGTCATTTTCATGGGACTGGTCTGGCCACAACT |
| | | ACATTAATGAAATATTTGCCACATCCTCTTACACTTTTTCATACATTGCCCAAGA |
| | | ATAAAG |
| 23 | Fiber | AGGGTATAACTCACCTGACAATCAGAGGGCGAGGTATTCAGCTCAACGACGA |
| | Deletion 2 | GTCGGTGAGCTCCTCGCTTGGTCTCCGTCCGGACGGGACATTTCAGATCGGC |
| | [Deleted | GGCGCCGGCCGCTCTTCATTCACGCCTCGTCAGGCAATCCTAACTCTGCAGA |
| | sequence | CCTCGTCCTCTGAGCCGCGCTCTGGAGGCATTGGAACTCTGCAATTTATTGA |
| | starting 5′ to | GGAGTTTGTGCCATCGGTCTACTTTAACCCCTTCTCGGGACCTCCCGGCCAC |
| | Fiber gene | TATCCGGATCAATTTATTCCTAACTTTGACGCGGTAAAGGACTCGGCGGACGG |
| | (3′region of | CTACGACTGAATGTTAAGTGGAGAGGCAGAGCAACTGCGCCTGAAACACCTG |
| | Hexon- | GTCCACTGTCGCCGCCACAAGTGCTTTGCCCGCGACTCCGGTGAGTTTTGCT |
| | associated | ACTTTGAATTGCCCGAGGATCATATCGAGGGCCCGGCGCACGGCGTCCGGC |
| | precursor/ | TTACCGCCCAGGGAGAGCTTGCCCGTAGCCTGATTCGGGAGTTTACCCAGCG |
| | pVIII), | CCCCCTGCTAGTTGAGCGGGACAGGGGACCCTGTGTTCTCACTGTGATTTGC |
| | encompassing | AACTGTCCTAACCCTGGATTACATCAAGATCCTCTAGTTAATTAACTAGAGTAC |
| | the entire | CCGGGGATOTTATTCCCTTTAACTAATAAAAAAAAATAATAAAGCATCACTTAC |
| | Fiber ORF | TTAAAATCAGTTAGCAAATTTCTGTCCAGTTTATTCAGCAGCACCTCCTTGCCC |
| | (alternate | TCCTCCCAGCTCTGGTATTGCAGCTTCCTCCTGGCTGCAAACTTTCTCCACAA |
| | sequence | TCTAAATGGAATGTCAGTTTCCTCCTGTTCCTGTCCATCCGCACCCACTATCTT |
| | deletion)] | CATGTTGTTGCAGATGAAGCGCGCAAGACCGTCTGAAGATACCTTCAACCCC |
| | | GTGTATCCATATGACACGGAAACCGGTCCTCCAACTGTGCCTTTTCTTACTCC |
| | | TCCCTTTGTATCCCCCAATGGGTTTCAAGAGAGTCCCCCTGGGGTACTCTCTT |
| | | TGCGCCTATCCGAACCTCTAGTTACCTCCAATGGCATGCTTGCGCTCAAAATG |
| | | GGCAACGGCCTCTCTCTGGACGAGGCCGGCAACCTTACCTCCCAAAATGTAA |
| | | CCACTGTGAGCCCACCTCTCAAAAAAACCAAGTCAAACATAAACCTGGAAATA |
| | | TCTGCACCCCTCACAGTTACCTCAGAAGCCCTAACTGTGGCTGCCGCCGCAC |
| | | CTCTAATGGTCGCGGGCAACACACTCACCATGCAATCACAGGCCCCGCTAAC |
| | | CGTGCACGACTCCAAACTTAGCATTGCCACCCAAGGACCCCTCACAGTGTCA |
| | | GAAGGAAAGCTAGCCCTGCAAACATCAGGCCCCCTCACCACCACCGATAGCA |
| | | GTACCCTTACTATCACTGCCTCACCCCCTCTAACTACTGCCACTGGTAGCTTG |
| | | GGCATTGACTTGAAAGAGCCCATTTATACACAAAATGGAAAACTAGGACTAAA |
| | | GTACGGGGCTCCTTTGCATGTAACAGACGACCTAAACACTTTGACCGTAGCAA |
| | | CTGGTCCAGGTGTGACTATTAATAATACTTCCTTGCAAACTAAAGTTACTGGAG |
| | | CCTTGGGTTTTGATTCACAAGGCAATATGCAACTTAATGTAGCAGGAGGACTA |
| | | AGGATTGATTCTCAAAACAGACGCCTTATACTTGATGTTAGTTATCCGTTTGAT |
| | | GCTCAAAACCAACTAAATCTAAGACTAGGACAGGGCCCTCTTTTTATAAACTCA |
| | | GCCCACAACTTGGATATTAACTACAACAAAGGCCTTTACTTGTTTACAGCTTCA |
| | | AACAATTCCAAAAAGCTTGAGGTTAACCTAAGCACTGCCAAGGGGTTGATGTT |
| | | TGACGCTACAGCCATAGCCATTAATGCAGGAGATGGGCTTGAATTTGGTTCAC |
| | | CTAATGCACCAAACACAAATCCCCTCAAAACAAAAATTGGCCATGGCCTAGAA |
| | | TTTGATTCAAACAAGGCTATGGTTCCTAAACTAGGAACTGGCCTTAGTTTTGAC |
| | | AGCACAGGTGCCATTACAGTAGGAAACAAAAATAATGATAAGCTAACTTTGTG |
| | | GACCACACCAGCTCCATCTCCTAACTGTAGACTAAATGCAGAGAAAGATGCTA |
| | | AACTCACTTTGGTCTTAACAAAATGTGGCAGTCAAATACTTGCTACAGTTTCAG |
| | | TTTTGGCTGTTAAAGGCAGTTTGGCTCCAATATCTGGAACAGTTCAAAGTGCT |
| | | CATCTTATTATAAGATTTGACGAAAATGGAGTGCTACTAAACAATTCCTTCCTG |
| | | GACCCAGAATATTGGAACTTTAGAAATGGAGATCTTACTGAAGGCACAGCCTA |
| | | TACAAACGCTGTTGGATTTATGCCTAACCTATCAGCTTATCCAAAATCTCACGG |
| | | TAAAACTGCCAAAAGTAACATTGTCAGTCAAGTTTACTTAAACGGAGACAAAAC |
| | | TAAACCTGTAACACTAACCATTACACTAAACGGTACACAGGAAACAGGAGACA |
| | | CAACTCCAAGTGCATACTCTATGTCATTTTCATGGGACTGGTCTGGCCACAAC |
| | | TACATTAATGAAATATTTGCCACATCCTCTTACACTTTTTCATACATTGCCCAAG |
| | | AATAAAGAATCGTTTGTGTTATGTTTCAACGTGTTTATTTTT |
| 24 | E4 deletion 1 | AATGTTGTCTCTACGCCTGCGGGTATGTATTCCCCCGGGCTATTTCGGTCGCT |
| | [Removes | TTTTAGCACTGACCGATGTGAATCAACCTGATGTGTTTACCGAGTCTTACATTA |
| | E4orf6 intron | TGACTCCGGACATGACCGAGGAGCTGTCGGTGGTGCTTTTTAATCACGGTGA |
| | sequence | CCAGTTTTTTTACGGTCACGCCGGCATGGCCGTAGTCCGTCTTATGCTTATAA |
| | containing | GGGTTGTTTTTCCTGTTGTAAGACAGGCTTCTAATGTTTAAATGTTTTTTTGTTA |
| | E4orf1, | TTTTATTTTGTGTTTATGCAGAAACCCGCAGACATGTTTGAGAGAAAAATGGTG |
| | E4orf2, | TCTTTTTCTGTGGTGGTTCCGGAGCTTACCTGCCTTTATCTGCATGAGCATGA |
| | E4orf3, | CTACGATGTGCTTTCTTTTTTGCGCGAGGCTTTGCCTGATTTTTTGAGCAGCA |
| | E4orf4 start | CCTTGCATTTTATATCGCCGCCCATGCAACAAGCTTACATCGGGGCTACGCTG |
| | codon, retain | GTTAGCATAGCTCCGAGTATGCGTGTCATAATCAGTGTGGGTTCTTTTGTCAT |
| | splice donor/ | GGTTCCTGGCGGGGAAGTGGCCGCGCTGGTCCGTGCAGACCTGCACGATTA |
| | splice | TGTTCAGCTGGCCCTGCGAAGGGACCTACGGGATCGCGGTATTTTTGTTAAT |
| | acceptor and | GTTCCGCTTTTGAATCTTATACAGGTCTGTGAGGAACCTGAATTTTTGCAATCA |
| | surrounding | TGATTCGCTGCTTGAGGCTGAAGGTGGAGGGCGCTCTGGAGCAGATTTTTAC |
| | sequences] | AATGGCCGGACTTAATATTCGGGATTTGCTTAGAGATATATTGAGAAGGTGGC |
| | | GAGATGAGAATTATTTGGGCATGGTTGAAGGTGCTGGAATGTTTATAGAGGAG |
| | | ATTCACCCTGAAGGGTTTAGCCTTTACGTCCACTTGGACGTGAGGGCCGTTTG |
| | | CCTTTTGGAAGCCATTGTGCAACATCTTACAAATGCCATTATCTGTTCTTTGGC |
| | | TGTAGAGTTTGACCACGCCACCGGAGGGGAGCGCGTTCACTTAATAGATCTT |
| | | CATTTTGAGGTTTTGGATAATCTTTTGGAATAAAAAAAAAAACATGGTTCTTCC |
| | | AGC |
| 25 | Fiber | TTAATGTAGTTGTGGCCAGACCAGTCCCATGAAAATGACATAGAGTATGCACT |
| | deletion 3 | TGGAGTTGTGTCTCCTGTTTCCTGTGTACCG |
| | [Remove | |
| | residual non- | |
| | coding Fiber | |
| | sequence] | |
| 26 | Fiber 5′ | GAAGCGCGCAAGACCG |
| | forward | |
| 27 | Fiber 5′ | CGGATAGGCGCAAAGAGAG |
| | reverse | |
| 28 | Fiber 5′ | ACGGAAACCGGTCCTCCAACTGTGCC |
| | probe | |
| 29 | Fiber 3′ | CGGAGACAAAACTAAACCTGTAACAC |
| | forward | |
| 30 | Fiber 3′ | TTGTGGCCAGACCAGTCC |
| | reverse | |
| 31 | Fiber 3′ | ACGGTACACAGGAAACAGGAGACACAACTCC |
| | probe | |
| 32 | E4orf6 | AGCGCGCGAATAAACTGC |
| | forward | |
| 33 | E4orf6 | TAAGTGAGATCAGGGTGCGC |
| | reverse | |
| 34 | E4orf6 probe | CGCTCCGTCCTGCAGGAATACAACAT |
| 35 | L3-23K | ATGGGCTCCAGTGAGCAGGAACTGAAAGCCATTGTCAAAGATCTTGGTTGTG |
| | endoprotease | GGCCATATTTTTTGGGCACCTATGACAAGCGCTTTCCAGGCTTTGTTTCTCCA |
| | Parent A | CACAAGCTCGCCTGCGCCATAGTCAATACGGCCGGTCGCGAGACTGGGGGC |
| | backbone | GTACACTGGATGGCCTTTGCCTGGAACCCGCACTCAAAAACATGCTACCTCTT |
| | | TGAGCCCTTTGGCTTTTCTGACCAGCGACTCAAGCAGGTTTACCAGTTTGAGT |
| | | ACGAGTCACTCCTGCGCCGTAGCGCCATTGCTTCTTCCCCCGACCGCTGTAT |
| | | AACGCTGGAAAAGTCCACCCAAAGCGTACAGGGGCCCAACTCGGCCGCCTG |
| | | TGGACTATTCTGCTGCATGTTTCTCCACGCCTTTGCCAACTGGCCCCAAACTC |
| | | CCATGGATCACAACCCCACCATGAACCTTATTACCGGGGTACCCAACTCCATG |
| | | CTCAACAGTCCCCAGGTACAGCCCACCCTGCGTCGCAACCAGGAACAGCTCT |
| | | ACAGCTTCCTGGAGCGCCACTCGCCCTACTTCCGCAGCCACAGTGCGCAGAT |
| | | TAGGAGCGCCACTTCTTTTTGTCACTTGAAAAACATGTAA |
| 36 | pIIIa | ATGATGCAAGACGCAACGGACCCGGCGGTGCGGGCGGCGCTGCAGAGCCA |
| | Parent A | GCCGTCCGGCCTTAACTCCACGGACGACTGGCGCCAGGTCATGGACCGCAT |
| | backbone | CATGTCGCTGACTGCGCGCAATCCTGACGCGTTCCGGCAGCAGCCGCAGGC |
| | | CAACCGGCTCTCCGCAATTCTGGAAGCGGTGGTCCCGGCGCGCGCAAACCC |
| | | CACGCACGAGAAGGTGCTGGCGATCGTAAACGCGCTGGCCGAAAACAGGGC |
| | | CATCCGGCCCGACGAGGCCGGCCTGGTCTACGACGCGCTGCTTCAGCGCGT |
| | | GGCTCGTTACAACAGCGGCAACGTGCAGACCAACCTGGACCGGCTGGTGGG |
| | | GGATGTGCGCGAGGCCGTGGCGCAGCGTGAGCGCGCGCAGCAGCAGGGCA |
| | | ACCTGGGCTCCATGGTTGCACTAAACGCCTTCCTGAGTACACAGCCCGCCAA |
| | | CGTGCCGCGGGGACAGGAGGACTACACCAACTTTGTGAGCGCACTGCGGCT |
| | | AATGGTGACTGAGACACCGCAAAGTGAGGTGTACCAGTCTGGGCCAGACTAT |
| | | TTTTTCCAGACCAGTAGACAAGGCCTGCAGACCGTAAACCTGAGCCAGGCTTT |
| | | CAAAAACTTGCAGGGGCTGTGGGGGGTGCGGGCTCCCACAGGCGACCGCGC |
| | | GACCGTGTCTAGCTTGCTGACGCCCAACTCGCGCCTGTTGCTGCTGCTAATA |
| | | GCGCCCTTCACGGACAGTGGCAGCGTGTCCCGGGACACATACCTAGGTCACT |
| | | TGCTGACACTGTACCGCGAGGCCATAGGTCAGGCGCATGTGGACGAGCATAC |
| | | TTTCCAGGAGATTACAAGTGTCAGCCGCGCGCTGGGGCAGGAGGACACGGG |
| | | CAGCCTGGAGGCAACCCTAAACTACCTGCTGACCAACCGGCGGCAGAAGATC |
| | | CCCTCGTTGCACAGTTTGCACCCTTTGGCGCATCCCATTCTCCAGTAA |

1. A polynucleotide comprising one or more adenoviral genes or genome regions, wherein one or more of the adenoviral genes or genome regions comprises one or more inactivating mutations, and wherein the one or more adenoviral genes or genome regions is selected from the group consisting of:
(a) an adenoviral fiber gene;
(b) an adenoviral precursor terminal protein (pTP) gene;
(c) an adenoviral L1-52K gene;
(d) an adenoviral 100K gene;
(e) an adenoviral PVIII gene;
(f) an adenoviral E4 region open reading frame (orf);
(g) an adenoviral inverted terminal repeat (ITR) sequence;
(h) an L3-23K region gene;
(i) a hexon-assembly gene; and
(j) a combination of any of (a)-(i).

2. The polynucleotide of claim 1, wherein the one or more inactivating mutations are selected from the group consisting of: a frame-shift, a start codon disruption, an internal start codon disruption, a stop codon insertion, a deletion, an insertion, an inversion, or any combination thereof.

3. The polynucleotide of claim 1 comprising (a) an inactivated adenoviral fiber gene.

4. The polynucleotide of claim 3, wherein the adenoviral fiber gene comprises a nucleic acid sequence at least 95% identical to the sequence of SEQ ID NO: 3.

5. The polynucleotide of claim 4, wherein the inactivated adenoviral fiber gene comprises one or more of the following inactivating mutations: (i) c.49delAT, (ii) c.1_2AT>TA, (iii) c.22G>T, (iv) c.45T>A, (v) c.196_199ATGG>TAG, (vi) c.361-362AT>TA, (vii) c.385-386AT>TA, (viii) c.751-752AT>TA, (ix) c.1118delT, (x) c.1654-1655AT>TA, and/or (xi) FiberDeletion3.

6. The polynucleotide of claim 1, wherein the one or more inactivating mutations is selected from the group consisting of:
(b) an inactivated adenoviral pTP gene, wherein the adenoviral pTP gene comprises a nucleic acid sequence at least 95% identical to SEQ ID NO: 5, and wherein the inactivated adenoviral pTP gene comprises a c.1T>A mutation and/or pTPDeletion1;
(c) an inactivated adenoviral L1-52K gene, wherein the adenoviral L1 protein gene comprises a nucleic acid sequence at least 95% identical to SEQ ID NO: 7, and wherein the inactivated adenoviral L1-52K gene comprises one or more of the following mutations: c.3G>A, L152KDeletion1, and/or L152KDeletion2;
(d) an inactivated adenoviral 100K gene, wherein the adenoviral 100K gene comprises a nucleic acid sequence at least 95% identical to SEQ ID NO: 10, and the inactivated adenoviral 100K gene comprises one or more of the following mutations: (i) c.1847T>A, (ii) c.2026A>T, (iii) c.2239_2240AT>TA, (iv) c.2519_2520AT>TA, (v) c.2685T>A, (vi) c.3G>T, (vii) c.312G>C, and/or (viii) c.480G>T;
(e) an inactivated adenoviral PVIII gene, wherein the adenoviral PVIII gene comprises a nucleic acid sequence at least 95% identical to SEQ ID NO: 13, and the inactivated adenoviral PVIII gene comprises one or more of the following mutations: (i) c.1T>A, (ii) c.25_29TACAT>ATCTA, (iii) c.49_50TA>AT, (iv) c.100 101AT>TA, and/or (v) PVIIIDeletion1;
(f) an inactivated adenoviral E4 region, wherein the adenoviral E4 region comprises one or more of the following mutations: (i) E4orf1 c.1_2TA>AT, (ii) E4orf2 c.1T>A, (iii) E4orf3 c.1_2TA>AT, (iv) E4orf4 c.1_2TA>AT, (v) E4orf1 c.1_3delATG, (vi) E4Deletion1, (vii) E4orf2 c.-18_-16delATG (upstream), (viii) E4orf2 c.1_3delATG, (ix) E4orf2 c.16_18delATG, (x) E4orf3 c.1_3delATG, (xi) E4orf3 c.55_57delATG, and/or (xii) E4orf4 c.1_3delATG;
(g) deletion of SEQ ID NO: 20 or SEQ ID NO: 21;
(h) an inactivated adenoviral L3-23K gene, wherein the L3-23K comprises a nucleic acid sequence at least 95% identical to SEQ ID NO: 35;
(i) an inactivated adenoviral hexon-assembly gene, wherein the adenoviral hexon-assembly gene comprises a nucleic acid sequence at least 95% identical to SEQ ID NO: 11, and the inactivated adenoviral hexon-assembly gene comprises one or more of the following mutations: (i) c.1_3delATG, (ii) c.933insTAATAA, and/or (iii) c.1937-1938delAT; and
any combination thereof.

7.-13. (canceled)

14. The polynucleotide of claim 1, comprising the inactivating mutations of Variant A4:
(a) an inactivated adenoviral fiber gene comprising the following mutations: c.1_2AT>TA, c.22G>T, c.45T>A, c.196_199ATGG>TAG, c.361-362AT>TA, c.385-386AT>TA, c.751-752AT>TA, and c.1118delT;
(f) an inactivated adenoviral E4 region and comprising the following mutations: c.1_2TA>AT (E4orf1), c.1T>A (E4orf2), c.1_2TA>AT (E4orf3), and c.1_2TA>AT (E4orf4); and
(g) ITRDeletion1.

15. The polynucleotide of claim 4, comprising the inactivating mutation of Variant A5: (a) an inactivated adenoviral fiber gene comprising a c.49delAT inactivation mutation.

16. The polynucleotide of claim 1, comprising the inactivating mutations of Variant A6, comprising:
(a) an inactivated adenoviral fiber gene and comprising the following mutations: c.1_2AT>TA, c.22G>T, c.45T>A, c.196_199ATGG>TAG, c.361-362AT>TA, c.385-386AT>TA, c.751-752AT>TA, c.1118delT, and c.1654-1655AT>TA;
(b) an inactivated adenoviral precursor terminal protein gene and comprising the following mutations: pTP Deletion 1 and c.1T>A;
(c) an inactivated adenoviral L1 region protein gene and comprising the following mutations: c.3G>A, L1 52K Deletion 1, and L1 52K Deletion 2;
(d) an inactivated adenoviral 100K protein gene and comprising the following mutations: c.1847T>A, c.2026A>T, c.2239_2240AT>TA, c.2519_2520AT>TA, and c.2685T>A;
(e) an inactivated adenoviral PVIII protein gene and comprising the following mutations: c.1T>A, c.25_29TACAT>ATCTA, c.49_50TA>AT, and c.100_101AT>TA;
(f) an inactivated adenoviral E4 region protein gene and comprising the following mutations: E4orf1 c.1_2TA>AT, E4orf2 c.1T>A, E4orf3 c.1_2TA>AT, E4orf4 c.1_2TA>AT; and
(g) ITRDeletion1.

17. The polynucleotide of claim 1, comprising the inactivating mutations of Variant A7, comprising:
(a) an inactivated adenoviral fiber gene and comprising the following mutations: c.1_2AT>TA, c.22G>T, c.45T>A, c.196_199ATGG>TAG, c.361-362AT>TA, c.385-386AT>TA, c.751-752AT>TA, c.1118delT, and c.1654-1655AT>TA;
(b) an inactivated adenoviral precursor terminal protein gene and comprising the following mutations: pTP Deletion 1 and c.1T>A;
(c) an inactivated adenoviral L1 region protein gene and comprising the following mutations: c.3G>A and L1 52K Deletion 1;
(d) an inactivated adenoviral 100K protein gene and comprising the following mutations: c.1847T>A, c.2026A>T, c.2239_2240AT>TA, c.2519_2520AT>TA, and c.2685T>A;
(e) an inactivated adenoviral PVIII protein gene and comprising the following mutations: c.1T>A, c.25_29TACAT>ATCTA, c.49_50TA>AT, and c.100_101AT>TA;
(f) an inactivated adenoviral E4 region protein gene and comprising the following mutations: c.1_2TA>AT and c.1T>A; and
(g) ITRDeletion1.

18. The polynucleotide of claim 1, comprising the inactivating mutations of Variant A8, comprising:
(a) an inactivated adenoviral fiber gene and comprising the following mutations: c.1_2AT>TA, c.22G>T, and c.45T>A;
(b) an inactivated adenoviral precursor terminal protein gene and comprising the following mutations: pTP Deletion 1 and c.1T>A;
(c) an inactivated adenoviral L1 region protein gene and comprising the following mutations: c.3G>A, L1 52K Deletion 1, and L1 52K Deletion 2;
(d) an inactivated adenoviral 100K protein gene and comprising the following mutations: c.1847T>A, c.2026A>T, c.2239_2240AT>TA, c.2519_2520AT>TA, and c.2685T>A; and
(e) an inactivated adenoviral PVIII protein gene and comprising the following mutations: c.1T>A, c.25_29TACAT>ATCTA, c.49_50TA>AT, and c.100_101AT>TA.

19. The polynucleotide of claim 1, comprising the inactivating mutations of Variant A9, comprising:
(a) an inactivated adenoviral fiber gene and comprising the following mutations: c.1_2AT>TA, c.22G>T, c.45T>A, c.196_199ATGG>TAG, c.361-362AT>TA, c.386-386AT>TA, c.751-752AT>TA, c.1118delT, and c.1654-1655AT>TA;
(b) an inactivated adenoviral precursor terminal protein gene and comprising the following mutations: pTP Deletion 1 and c.1T>A;
(c) an inactivated adenoviral L1 region protein gene and comprising the following mutations: c.3G>A, L1 52K Deletion 1, and L1 52K Deletion 2;
(f) an inactivated adenoviral E4 region protein gene and comprising the following mutations: c.1_2TA>AT (E4orf1), c.1T>A (E4orf2), c.1_2TA>AT (E4orf3), and c.1_2TA>AT (E4orf4); and
(g) ITRDeletion1.

20. The polynucleotide of claim 1, comprising the inactivating mutations of Variant B1, comprising:
(d) an inactivated adenoviral 100K protein gene and comprising the following mutations: c.3G>T, c.312G>C, and c.480G>T;
(e) an inactivated adenoviral PVIII protein gene and comprising the mutation pVIII Deletion 1;
(f) an inactivated adenoviral E4 region protein gene and comprising the following mutations: E4orf1 c.1_3delATG, E4orf2 c.-18_-16delATG (upstream), E4orf2 c.1_3delATG, E4orf2 c.16_18delATG, E4orf3 c.1_3delATG, E4orf3 c.55_57delATG, and E4orf4 c.1_3delATG; and
(i) an inactivated hexon assembly gene and comprising the mutations c.1_3delATG, c.933insTAATAA, and c.1937-1938delAT.

21. The polynucleotide of claim 1, comprising the inactivating mutations of Variant B2, comprising:
(a) an inactivated adenoviral fiber gene and comprising the mutation Fiber deletion 3;
(d) an inactivated adenoviral 100K protein gene and comprising the following mutations: c.3G>T, c.312G>C, and c.480G>T;
(e) an inactivated adenoviral PVIII protein gene and comprising the mutation pVIII Deletion 1;
(f) an inactivated adenoviral E4 region protein gene and comprising the mutation E4 deletion 1; and
(i) an inactivated hexon assembly gene and comprising the mutations c.1_3delATG, c.933insTAATAA, and c.1937-1938delAT.

22. The polynucleotide of claim 1, wherein the adenovirus genes are adenovirus serotype 5 adenovirus genes.

23. The polynucleotide of claim 1, wherein the adenovirus genes are adenovirus serotype 2 adenovirus genes.

24. The polynucleotide of claim 1, where the polynucleotide is an AAV helper plasmid.

25.-29. (canceled)

30. A host cell comprising the polynucleotide of claim 1.

31. (canceled)

32. A method of producing recombinant AAV, the method comprising culturing the host cell of claim 30 and isolating recombinant AAV.

33.-37. (canceled)
